(19) United States
(12) Patent Application Publication
(10) Pub. No.: US 2019/0237189 A1
(43) Pub. Date: Aug. 1, 2019

(54) PERIOPERATIVE WORKFLOW SYSTEM, ARCHITECTURE, AND INTERFACE THERETO

(71) Applicant: Logical Medical Systems, Inc., Los Angeles, CA (US)

(72) Inventors: David Geller, Los Angeles, CA (US); Edwin Pankau, Los Angeles, CA (US); Padgett Arango, PORTLAND, OR (US); Andre Clark, Los Angeles, CA (US); Dean Nakabayashi, SAN FRANCISCO, CA (US); Moise Danielpour, LOS ANGELES, CA (US); Christopher Shattuck, Litchfield, CT (US)

(21) Appl. No.: 16/367,559

(22) Filed: Mar. 28, 2019

Related U.S. Application Data
(63) Continuation of application No. PCT/US2017/056676, filed on Oct. 13, 2017.
(60) Provisional application No. 62/410,390, filed on Oct. 20, 2016.

(51) Int. Cl. G16H 40/20; G06Q 10/06; G06F 3/0482

(52) U.S. Cl. CPC G16H 40/20; G06F 3/0482; G06Q 10/0633

(57) ABSTRACT

A system supporting perioperative workflow includes a backend system with at least one database; at least one application configured with the database(s); and at least one user interface (UI) mechanism supporting a plurality of role-specific graphical user interfaces (GUIs) to the backend system. The backend system maintains an authoritative version of perioperative workflow information for each of a plurality of patients. One or more devices are configured with at least some of the role-specific GUIs, and operably connected to and interact with the backend system via the UI mechanism(s). Each device is configured to: receive and display perioperative workflow information from the backend system in a role-specific GUI on the device; and to send perioperative workflow information to the backend system via the role-specific GUI on the device.

### RELATED APPLICATIONS

This application is a continuation of PCT/US2017/056676, filed Oct. 13, 2017, which is related to and claims priority from U.S. Provisional Patent Application No. 62/410,390, titled “Perioperative Workflow System, Architecture, And Interface Thereto,” and filed Oct. 20, 2016, the entire contents of both of which are hereby fully incorporated herein by reference for all purposes.

### BACKGROUND OF THE INVENTION

### Copyright Statement

This patent document contains material subject to copyright protection. The copyright owner has no objection to the reproduction of this patent document or any related materials in the files of the United States Patent and Trademark Office, but otherwise reserves all copyrights whatsoever.

### FIELD OF THE INVENTION

This invention relates to improving workflow in medical systems and environments, and, more specifically to perioperative workflows, and systems, frameworks, architectures, and devices supporting efficient and safe perioperative workflows.

### BACKGROUND

Operating rooms (ORs) represent a hospital's single biggest profit center and its most expensive area to maintain. According to The Journal of the American Society of Anesthesiologists, Inc., services related to surgery can represent more than 40% of hospital costs and revenues, with the largest hospital cost category being the operating room (33%). [Macario, et al, “Where Are the Costs in Perioperative Care?: Analysis of Hospital Costs and Charges for Inpatient Surgical Care.” Anesthesiology 1995; 83(6):1138-1144.] Media Healthleaders magazine reports that as much as 60% to 70% of hospital revenues are tied to the operating room. [Cantlupe, J. “Anesthesiology Focus for Operating Room Efficiency,” Dec. 26, 2012.]

Becker's Hospital Review notes that “[t]ime is an OR's most valuable resource. Even a slight delay in a case's start time, a lengthy turnover, or a few minutes spent looking for a piece of missing equipment, can severely hinder an OR's efficiency and ability to maintain a positive contribution margin.” [Gamble, M. “6 Cornerstones of Operating Room Efficiency: Best Practices for Each,” Becker's Hospital Review Jan. 18, 2013.]

Based on the importance of the operating room, many hospitals embrace technological advances in the hopes of improving operating room efficiency and reducing turnover time. Even the smallest gain in operating room efficiency can have a direct impact on a hospital's bottom line. This is an especially important point given the fact that many leading hospitals' operating rooms, particularly in nonprofit teaching hospitals, are overscheduled with lengthy and variable turnover times. In order to maintain and improve efficiency, an entire surgical team must continually adapt, including leveraging the latest surgical instruments and minimally invasive techniques, or relying on new hardware and software to access real-time patient data and digital images, quickly track assets, or communicate with team members. Still, independent of the specific technological advances a particular hospital may leverage in order to accomplish or streamline operating room-related tasks, one aspect of surgical care is virtually the same at every hospital in the world: the perioperative team and workflow. The term “perioperative,” as used herein, has its normal meaning, and generally refers to the three phases of surgery: preoperative, intraoperative, and postoperative.

Worldwide, the perioperative team, consisting of nurses, surgeons, residents, anesthesiologists, cleaning crew, and technicians, follows a nearly identical workflow. This uniformity makes sense given that the composition of the surgical team and the sequence of steps in the perioperative workflow are based on global standards establishing best practices for patient and staff safety. In addition, keeping the perioperative flow consistent across hospitals enables surgeons and other team members to adjust quickly to new institutions, thus making the introduction and contributions of new surgical team members more efficient.

Stakeholders in the perioperative workflow are faced with many serious and complex tasks throughout the day and must also remain constantly aware of where a patient is within the perioperative flow so that they do not become a bottleneck. Thus, team members must continually check in with various staff, fixed overhead screens, control room boards, or the like in order to confirm whether or not pertinent steps of the flow have been completed and/or if the schedule for their own tasks has changed. Often, stakeholders are unaware of the timing for when they will be needed and/or receive little to no advance warning. It is not uncommon to find that people are not readily available when other team members contact them. Similarly, when timely requests are made from the operating room, such as for a technician or cleaning crew, the requesting party may not have insight into whether or not a request was received and/or timing for when a request will be fulfilled.

It is desirable, and an object hereof, to take the guesswork out of the perioperative workflow, such that the focus of hospital staff can remain on the more important perioperative tasks rather than on figuring out where and when a given task needs to be performed.

It is desirable, and an object hereof, to improve the efficiency of the all-important perioperative workflow.

### SUMMARY

The present invention is specified in the claims as well as in the description.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a system supporting perioperative workflow, the system including: (A) a backend system including: (A)(1) at least one database; (A)(2) at least one application configured with the at least one database; and (A)(3) at least one user interface mechanism supporting a plurality of role-specific graphical user interfaces (GUIs) to the backend system. The backend system may maintain in the at least one database, perioperative workflow information for each of a plurality of patients, and where the perioperative workflow information maintained by the backend system is an authoritative version of the perioperative workflow information. The system may include one or more devices configured with at least some of the plurality of role-specific GUIs, and operably connected to the backend system and interacting with the backend system via the at least one user interface mechanism. According to some aspects, each particular device of the one or more devices is configured to: (B)(1) receive and display perioperative workflow information from the backend system in the role-specific GUI on the particular device; and (B)(2) send perioperative workflow information to the backend system via the role-specific GUI on the particular device.

Other embodiments of this aspect include corresponding computer systems, apparatus, and computer programs recorded on one or more computer storage devices, each configured to perform the actions of the methods.

Implementations and/or embodiments may include one or more of the following features:
The system where the perioperative workflow information in the at least one database includes a sequence of perioperative workflow steps.
The system where the sequence of perioperative workflow steps include synchronous steps and asynchronous steps.
The system where the sequence of perioperative workflow steps are selected from: user registration, user login, patient information entry, viewing patient information, viewing patient status, patient scheduling, doctor information entry, viewing doctor information, doctor scheduling, requesting doctor, procedure information entry, viewing procedure information, scheduling procedure, requesting procedure, non-operating room (OR) nurse information entry, viewing non-OR nurse information, scheduling non-OR nurse, requesting non-OR nurse, OR nurse information entry, viewing OR nurse information, scheduling OR nurse, requesting OR nurse, anesthesiologist information entry, viewing anesthesiologist information, scheduling anesthesiologist, requesting anesthesiologist, tech information entry, viewing tech information, scheduling tech, requesting tech, tech request information entry, viewing tech request information, scheduling tech request, environmental services information entry, viewing environmental services information, scheduling environmental services, requesting environmental services, requesting blood, lab requests, transport, and room scheduling.
The system where the system supports a plurality of user roles, and where each particular user role has a corresponding role-specific GUI associated therewith.
The system where the role-specific GUI associated with each particular user role provides role-specific capabilities and role-specific permissions, and where the backend system enforces the role-specific capabilities and the role-specific permissions via the role-specific GUIs.
The system where the role-specific capabilities and the role-specific permissions include permission to view certain perioperative workflow information; and permission to modify or delete certain perioperative workflow information.
The system where, for certain roles, the permission to view certain perioperative workflow information includes permission to view certain perioperative workflow information associated with one or more specific patients. The system of any one −8 where the plurality of user roles are selected from the group: administrator, service manager, non-operating room (OR) nurse, doctor, anesthesiologist, OR nurse, tech, and environmental services.
The system where a user role is administrator and where the at least one role-specific GUI includes an administrative GUI that sends certain perioperative workflow information to the backend system.
The system where the certain perioperative workflow information is selected from: user detail information, login information, patients detail information, room information, doctors information, service request information, report information, tech request information, lab information, transport information, and blood request information. The system of any one −8, where each of the one or more devices is selected from: a mobile phone, a tablet computer, a desktop computer, and a laptop computer.
The system where, for certain roles, the permission to modify or delete certain perioperative workflow information includes permission to modify or delete certain perioperative workflow information associated with one or more specific patients.
The system where the role-specific permissions are selected from: administrator permissions, service manager permissions, non-OR nurse permissions, doctor permissions, anesthesiologist permissions, OR nurse permissions, tech permissions, and environmental services permissions.
The system where each specific patient of the plurality of patients has a corresponding specific perioperative workflow.
The system where the perioperative workflow associated with each particular patient is initially based on an expected treatment or procedure for the particular patient.
The system where the at least one application includes a scheduling application and a workflow application, and where, for each specific patient of the plurality of patients, the scheduling application and the workflow application: (a) monitor the specific patient's flow through the perioperative workflow associated with the specific patient; and (b) adjust the perioperative workflow associated with the specific patient based on: (b)(1) perioperative workflow information maintained in the at least one database at the backend system; and (b)(2) perioperative workflow information modified or deleted via the role-specific GUIs on the one or more devices.
The system where the scheduling application and the workflow application adjust to real-time variability of each step in the perioperative workflow associated with the specific patient.
The system where at least some of the role-specific GUIs provide a real-time view into steps within the perioperative workflow associated with the specific patient.
The system where the perioperative workflow associated with the specific patient includes synchronous steps and asynchronous steps.
The system where the at least one application includes: a data evaluation application configured to analyze at least one perioperative workflow and to generate at least one report based on the analysis.
The system where the at least one report is used to modify aspects of the at least one perioperative workflow.
The system where the aspects of the at least one perioperative workflow includes at least one sequence of perioperative workflow steps.
The system where the at least one application includes one or more of: a configuration application, an administration application, a perioperative workflow scheduling application, a perioperative workflow application, an intake application, an output application, and a data evaluation application.
The system where the at least one database includes one or more of: a perioperative workflow scheduling database, a configuration database, a general and administrative database, and a perioperative workflow information database.
The system where each role-specific GUI displays perioperative workflow information in a corresponding role-specific manner.
The system where displayed perioperative workflow information includes one or more of: user information, login information, patient information, room information, doctor information, service request information, report information, tech request information, lab information, transport information, and blood request information.
The system where the at least one role-specific GUI includes a service manager flow GUI that displays certain perioperative workflow information received from the backend system.
The system where the displayed perioperative workflow information is selected from: service requests information, room information, and account information.
The system where the at least one role-specific GUI includes a service manager GUI that sends perioperative workflow information to the backend system.
The system where the sent perioperative workflow information is selected from service request information, rooms information, and accounts information.
The system where the at least one role-specific GUI includes a non-OR nurse flow GUI that displays perioperative workflow information received from the backend system.
The system where the displayed perioperative workflow information is selected from: login information, patient information, history information, request information, room information, and account information.
The system where the at least one role-specific GUI includes a non-OR nurse flow GUI that sends certain perioperative workflow information to the backend system.
The system where the certain perioperative workflow information is selected from: login information, patient information, history information, request information, rooms information, and account information.
The system where the certain perioperative workflow information is selected from: patient information, procedure information, room information, service request information, tech request information, blood request information, history information, lab information, transport information, and account information.
The system where the at least one role-specific GUI is selected from: a doctor flow GUI, an anesthesiologist flow GUI and an OR nurse flow GUI, that each display certain perioperative workflow information received from the backend system.
The system where the at least one role-specific GUI is selected from: a doctor flow GUI, an anesthesiologist flow GUI, and an OR nurse flow interface, each of which sends certain perioperative workflow information to the backend system.
The system where the certain perioperative workflow information is selected from: patient information, procedure information, room information, service request information, tech request information, blood request information, history information, lab information, transport information, and accounts information.
The system where the at least one role-specific GUI includes a tech flow GUI that displays certain perioperative workflow information received from the backend system and sends perioperative workflow information to the backend system.
The system where the certain perioperative workflow information displayed by the tech flow GUI is selected from: tech request information, room information, and account information.
The system where the at least one role-specific GUI includes an environmental services flow GUI that displays certain perioperative workflow information received from the backend system and sends perioperative workflow information to the backend system.
The system where the displayed certain perioperative workflow information is selected from: request information, room information, and account information.
The system where the at least one application includes an intake application configured to receive information from an external system, and an output application configured to send information to an external system.
The system where the backend system is configured to generate reports based on stored perioperative information.
The system where the backend system is configured to determine efficiency of a particular perioperative process.
The system where the at least one application is configured to track synchronous and asynchronous steps required in the sequence associated with a particular perioperative workflow associated with a particular patient.
The system where the at least one application monitors the particular patient flow through the system.

Implementations of the described techniques may include hardware, a method or process, or computer software on a computer-accessible medium.

Another general aspect includes a method, in a system including: (A) a backend system having: (A)(1) at least one database; (A)(2) at least one application configured with the at least one database; and (A)(3) at least one user interface mechanism supporting a plurality of role-specific graphical user interfaces (GUIs) to the backend system, (B) one or more devices configured with at least some of the plurality of role-specific GUIs, and operably connected to the backend system and interacting with the backend system via the at least one user interface mechanism, where each particular device of the one or more devices is configured to: (B)(1) receive and display perioperative workflow information from the backend system in the role-specific GUI on the particular device; and (B)(2) send perioperative workflow information to the backend system via the role-specific GUI on the particular device, the method including: (a) maintaining in the at least one database, perioperative workflow information for each of a plurality of patients, and where the perioperative workflow information maintained by the backend system is an authoritative version of the perioperative workflow information; and (b) for each specific patient of the plurality of patients, (b)(1) monitoring the specific patient's flow through the perioperative workflow associated with the specific patient; and (b)(2) adjusting the perioperative workflow associated with the specific patient based on: (b)(1) perioperative workflow information maintained in the at least one database at the backend system; and (b)(2) perioperative workflow information modified or deleted via the role-specific GUIs on the one or more devices.

Other embodiments of this aspect include corresponding computer systems, apparatus, and computer programs recorded on one or more computer storage devices, each configured to perform the actions of the methods.

Another general aspect includes a method, in any of the system aspects described above, the method including: (a) maintaining in the at least one database, perioperative workflow information for each of a plurality of patients, and where the perioperative workflow information maintained by the backend system is an authoritative version of the perioperative workflow information; and (b) for each specific patient of the plurality of patients, (b)(1) monitoring the specific patient's flow through the perioperative workflow associated with the specific patient; and (b)(2) adjusting the perioperative workflow associated with the specific patient based on: (b)(1) perioperative workflow information maintained in the at least one database at the backend system; and (b)(2) perioperative workflow information modified or deleted via the role-specific GUIs on the one or more devices.

Below is a list of system embodiments. Those will be indicated with a letter “S”. Whenever such embodiments are referred to, this will be done by referring to “S” embodiments.
S1. A system supporting perioperative workflow, the system comprising:
(A) a backend system comprising:
   (A)(1) at least one database;
   (A)(2) at least one application configured with said at least one database; and
   (A)(3) at least one user interface mechanism supporting a plurality of role-specific graphical user interfaces (GUIs) to said backend system, wherein the backend system maintains in said at least one database, perioperative workflow information for each of a plurality of patients, and wherein the perioperative workflow information maintained by the backend system is an authoritative version of the perioperative workflow information; and
(B) one or more devices configured with at least some of the plurality of role-specific GUIs, and operably connected to said backend system and interacting with said backend system via said at least one user interface mechanism, wherein each particular device of said one or more devices is configured to:
   (B)(1) receive and display perioperative workflow information from said backend system in said role-specific GUI on said particular device;
and
(B)(2) send perioperative workflow information to said backend system via said role-specific GUI on said particular device.
S2. The system as in S1, wherein said perioperative workflow information in said at least one database comprises a sequence of perioperative workflow steps.
S3. The system of any of aspects S1-S2, wherein said sequence of perioperative workflow steps comprise synchronous steps and asynchronous steps.
S4. The system of any of S1-S3, wherein said system supports a plurality of user roles, and wherein each particular user role has a corresponding role-specific GUI associated therewith.
S5. The system of S4, wherein the role-specific GUI associated with each particular user role provides role-specific capabilities and role-specific permissions, and wherein said backend system enforces said role-specific capabilities and said role-specific permissions via said role-specific GUIs.
S6. The system of 5S, wherein the role-specific capabilities and the role-specific permissions include permission to view certain perioperative workflow information; and permission to modify or delete certain perioperative workflow information.
S7. The system of S6, wherein, for certain roles, said permission to view certain perioperative workflow information comprises permission to view certain perioperative workflow information associated with one or more specific patients.
S8. The system of any of S6-S7, wherein, for certain roles, said permission to modify or delete certain perioperative workflow information comprises permission to modify or delete certain perioperative workflow information associated with one or more specific patients.
S9. The system of any of S4-S8 wherein said plurality of user roles are selected from the group: administrator, service manager, non-operating room (OR) nurse, doctor, anesthesiologist, OR nurse, tech, and environmental services.
S10. The system of any of S1-S9, wherein each specific patient of said plurality of patients has a corresponding specific perioperative workflow.
S11. The system of any of S1-S10, wherein the perioperative workflow associated with each particular patient is initially based on an expected treatment or procedure for said particular patient.
S12. The system of any of S1-511, wherein said at least one application comprises a scheduling application and a workflow application, and wherein, for each specific patient of said plurality of patients, said scheduling application and said workflow application:
   (a) monitor said specific patient's flow through the perioperative workflow associated with said specific patient; and
   (b) adjust said perioperative workflow associated with said specific patient based on:
      (b)(1) perioperative workflow information maintained in said at least one database at said backend system; and/or
      (b)(2) perioperative workflow information modified or deleted via said role-specific GUIs on said one or more devices.
S13. The system of any of S10-S12, wherein said perioperative workflow associated with said specific patient comprises synchronous steps and asynchronous steps.
S14. The system of any of S1-S12, wherein the scheduling application and the workflow application adjust to real-time variability of each step in said perioperative workflow associated with said specific patient.
S15. The system of any of S1-S14, wherein at least some of the role-specific GUIs provide a real-time view into steps within the perioperative workflow associated with said specific patient.
S16. The system of any of S2-S15, wherein said sequence of perioperative workflow steps are selected from: user registration, user login, patient information entry, viewing patient information, viewing patient status, patient scheduling, doctor information entry, viewing doctor information, doctor scheduling, requesting doctor, procedure information entry, viewing procedure information, scheduling procedure, requesting procedure, non-operating room (OR) nurse information entry, viewing non-OR nurse information, scheduling non-OR nurse, requesting non-OR nurse, OR nurse information entry, viewing OR nurse information, scheduling OR nurse, requesting OR nurse, anesthesiologist information entry, viewing anesthesiologist information, scheduling anesthesiologist, requesting anesthesiologist, tech information entry, viewing tech information, scheduling tech, requesting tech, tech request information entry, viewing tech request information, scheduling tech request, environmental services information entry, viewing environmental services information, scheduling environmental services, requesting environmental services, requesting blood, lab requests, transport, and room scheduling.
S17. The system of any of S5-S16, wherein said role-specific permissions are selected from: administrator permissions, service manager permissions, non-OR nurse permissions, doctor permissions, anesthesiologist permissions, OR nurse permissions, tech permissions, and environmental services permissions.
S18. The system of any of S1-S16, wherein said at least one application comprises: a data evaluation application configured to analyze at least one perioperative workflow and to generate at least one report based on the analysis.
S19. The system of S18, wherein said at least one report and/or said analysis is used to modify aspects of said at least one perioperative workflow.
S20. The system of any of S1-S19, wherein said aspects of said at least one perioperative workflow includes at least one sequence of perioperative workflow steps.
S21. The system of any of S1-S20, wherein the at least one application comprises one or more of: a configuration application, an administration application, a perioperative workflow scheduling application, a perioperative workflow application, an intake application, an output application, and a data evaluation application.
S22. The system of any of S1-S21, wherein the at least one database comprises one or more of: a perioperative workflow scheduling database, a configuration database, a general and administrative database, and a perioperative workflow information database.
S23. The system of any of S1-S22, wherein each role-specific GUI displays perioperative workflow information in a corresponding role-specific manner.
S24. The system of any of S1-S23, wherein displayed perioperative workflow information comprises one or more of: user information, login information, patient information, room information, doctor information, service request information, report information, tech request information, lab information, transport information, and blood request information.
S25. The system of any of S1-S24, wherein a user role is administrator and wherein the at least one role-specific GUI includes an administrative GUI that sends certain perioperative workflow information to said backend system.
S26. The system of S25, wherein said certain perioperative workflow information is selected from: user detail information, login information, patients detail information, room information, doctors information, service request information, report information, tech request information, lab information, transport information, and blood request information.
S27. The system of any of S1-S26, wherein the at least one role-specific GUI includes a service manager flow GUI that displays certain perioperative workflow information received from said backend system.
S28. The system of S27, wherein said displayed perioperative workflow information is selected from: service requests information, room information, and account information.
S29. The system of any of S1-S28, wherein the at least one role-specific GUI includes a service manager GUI that sends perioperative workflow information to said backend system.
S30. The system of S29, wherein said sent perioperative workflow information is selected from service request information, rooms information, and accounts information.
S31. The system of any of S1-S30, wherein the at least one role-specific GUI includes a non-OR nurse flow GUI that displays perioperative workflow information received from said backend system.
S32. The system of S31, wherein said displayed perioperative workflow information is selected from: login information, patient information, history information, request information, room information, and account information.
S33. The system of any of S1-S32, wherein the at least one role-specific GUI includes a non-OR nurse flow GUI that sends certain perioperative workflow information to said backend system.
S34. The system of S33, wherein said certain perioperative workflow information is selected from: login information, patient information, history information, request information, rooms information, and account information.
S35. The system of any of S1-S34, wherein the at least one role-specific GUI is selected from: a doctor flow GUI, an anesthesiologist flow GUI and an OR nurse flow GUI, that each display certain perioperative workflow information received from said backend system.
S36. The system of S35, wherein said certain perioperative workflow information is selected from: patient information, procedure information, room information, service request information, tech request information, blood request information, history information, lab information, transport information, and account information.
S37. The system of any of S1-S36, wherein the at least one role-specific GUI is selected from: a doctor flow GUI, an anesthesiologist flow GUI, and an OR nurse flow interface, each of which sends certain perioperative workflow information to said backend system.
S38. The system of S37, wherein said certain perioperative workflow information is selected from: patient information, procedure information, room information, service request information, tech request information, blood request information, history information, lab information, transport information, and accounts information.
S39. The system of any of S1-S38, wherein the at least one role-specific GUI includes a tech flow GUI that displays certain perioperative workflow information received from said backend system and sends perioperative workflow information to said backend system.
S40. The system of S39, wherein said certain perioperative workflow information displayed by said tech flow GUI is selected from: tech request information, room information, and account information.
S41. The system of any of S1-S40, wherein the at least one role-specific GUI includes an environmental services flow GUI that displays certain perioperative workflow information received from said backend system and sends perioperative workflow information to said backend system.
S42. The system of S41, wherein said displayed certain perioperative workflow information is selected from: request information, room information, and account information.
S43. The system of any of S1-S42, wherein said at least one application includes an intake application configured to receive information from an external system, and an output application configured to send information to an external system.
S44. The system of any of S1-S43, wherein the backend system is configured to generate reports based on stored perioperative information.
S45. The system of any of S1-S44, wherein the backend system is configured to determine efficiency of a particular perioperative process.
S46. The system of any of S1-S45, wherein said at least one application is configured to track synchronous and asynchronous steps required in the sequence associated with a particular perioperative workflow associated with a particular patient.
S47. The system of any of S1-S46, wherein said at least one application monitors the particular patient flow through the system.
S48. The system of any one of the preceding system aspects S1-S47, wherein each of said one or more devices is selected from: a mobile phone, a tablet computer, a desktop computer, and a laptop computer.

Below is a list of method or process embodiments. Those will be indicated with a letter “M”. Whenever such embodiments are referred to, this will be done by referring to “M” embodiments.
M49. A method, in a system according to any of the system aspects S1-S48, the method comprising:
   (a) maintaining in said at least one database, perioperative workflow information for each of a plurality of patients, and wherein the perioperative workflow information maintained by the backend system is an authoritative version of the perioperative workflow information; and
   (b) for each specific patient of said plurality of patients,
      (b)(1) monitoring said specific patient's flow through the perioperative workflow associated with said specific patient; and
      (b)(2) adjusting said perioperative workflow associated with said specific patient based on:
         (b)(2)(i) perioperative workflow information maintained in said at least one database at said backend system; and/or
         (b)(2)(ii) perioperative workflow information modified or deleted via said role-specific GUIs on said one or more devices.

Below are computer-readable medium embodiments. Those will be indicated with a letter “C”.
C50. A non-transitory computer-readable medium with one or more computer programs stored therein that, when executed by one or more processors in a system according to any of the system aspects S1-S48, cause the one or more processors to perform at least the operations of the method M49.

The above features along are intended to illustrate aspects of the invention but are not intended to limit its scope in any way.

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification. None of the drawings is to scale unless specifically stated otherwise.

FIGS. 1-2 show overviews of aspects of a perioperative workflow framework in accordance with exemplary embodiments hereof;

FIGS. 3A-3D depict aspects of devices in accordance with exemplary embodiments hereof;

FIGS. 4A-4E depict aspects of computing and computer devices in accordance with exemplary embodiments hereof; and

FIGS. 5A-5X, 6A-6H, 7A-7T, 8A-8V, 9A-9X, 10A-10P, 11A-11G and 12A-12I are sample screens of an exemplary graphical user interface to a perioperative workflow framework in accordance with embodiments hereof.

### DETAILED DESCRIPTION OF THE PRESENTLY PREFERRED EXEMPLARY EMBODIMENTS

### Glossary and Abbreviations

As used herein, unless used otherwise, the following terms or abbreviations have the following meanings:

API means application programming interface;

GUI means graphical user interface;

UI means user interface; and

OR means operating room.

The term “mechanism,” as used herein, refers to any device(s), process(es), service(s), or combination thereof. A mechanism may be implemented in hardware, software, firmware, using a special-purpose device, or any combination thereof. A mechanism may be integrated into a single device or it may be distributed over multiple devices. The various components of a mechanism may be co-located or distributed. The mechanism may be formed from other mechanisms. In general, as used herein, the term “mechanism” may thus be considered shorthand for the term device(s) and/or process(es) and/or service(s).

### Overview

Overview—Structure

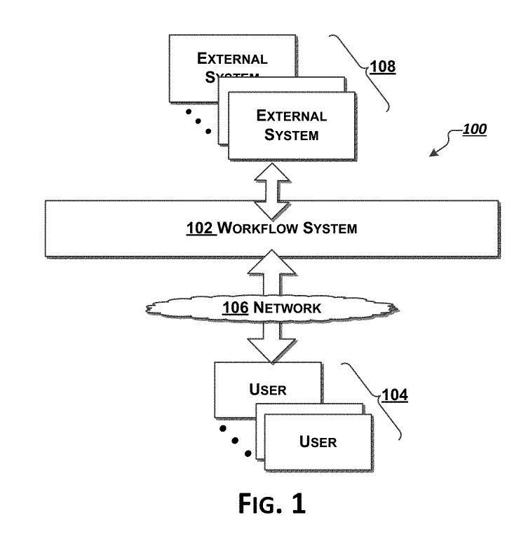

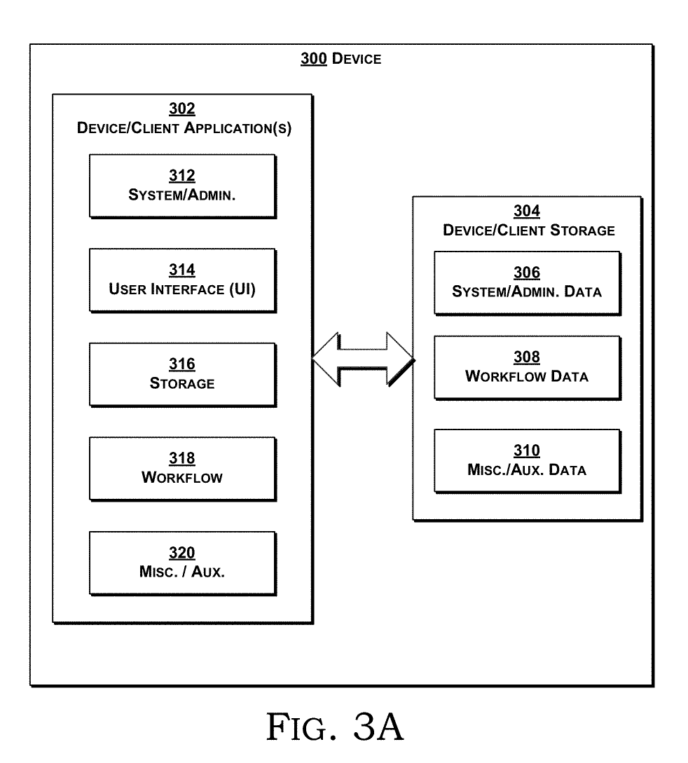

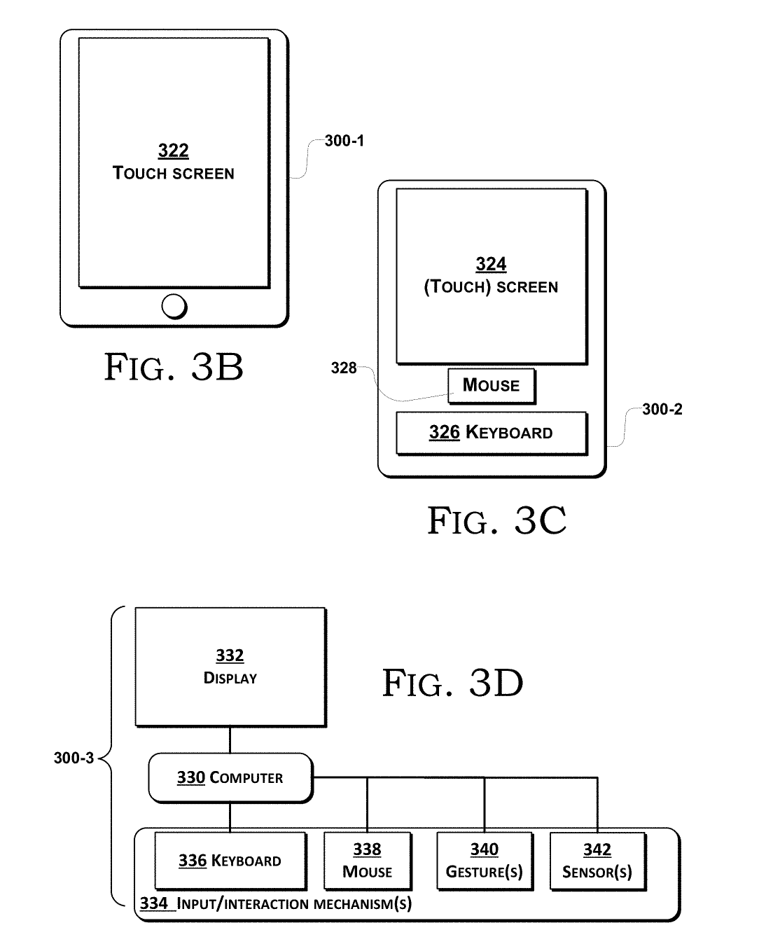

FIG. 1 shows an overview of an exemplary framework 100 for perioperative workflow according to exemplary embodiments hereof. As shown in FIG. 1, a perioperative workflow system 102 may be accessed by multiple users 104, e.g., via one or more networks 106 (e.g., the Internet). Each user 104 (e.g., a medical practitioner) may access the perioperative workflow system 102 using one or more computing devices, as discussed below with reference to FIGS. 3A-3D. The perioperative workflow system 102 may also access and be accessible by various external systems 108 (e.g., billing/accounting systems, external databases, and the like).

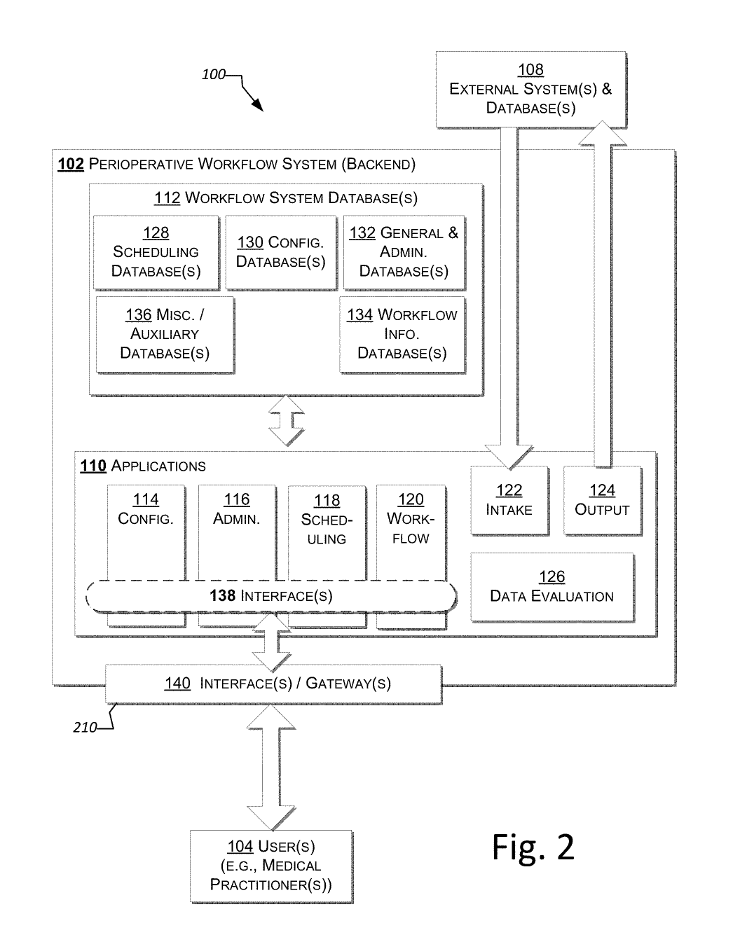

FIG. 2 shows aspects of the exemplary perioperative workflow framework 100 of FIG. 1. As shown in FIG. 2, the perioperative workflow system 102 (also sometimes referred to conveniently as the “backend”) comprises various applications 110 and one or more databases 112, described in greater detail below.

The database(s) 112 may be or comprise multiple separate or integrated databases, at least some of which may be distributed. The database(s) 112 may be implemented in any manner, and, when made up of more than one database, the various databases need not all be implemented in the same manner. It should be appreciated that the system is not limited by the nature or location of database(s) 112 or by the manner in which they are implemented.

Each of the applications 110 is essentially a mechanism (as defined above) that may provide one or more services via an appropriate interface. Although shown as separate mechanisms for the sake of this description, it should be appreciated that some or all of the various applications 110 may be combined. The various applications (mechanisms) 110 may be implemented in any manner and need not all be implemented in the same manner (e.g., with the same languages or interfaces or protocols).

The applications 110 may include configuration application(s) 114, administrative application(s) 116, perioperative workflow scheduling application(s) 118, perioperative workflow application(s) 120, intake application(s) 122, output application(s) 124, and data evaluation application(s) 126. The applications 110 may also include other miscellaneous and auxiliary applications (not shown).

The database(s) 112 may include perioperative workflow scheduling database(s) 128, configuration database(s) 130, general and administrative database(s) 132, perioperative workflow information database(s) 134, and miscellaneous and auxiliary database(s) 136.

As shown in the drawing in FIG. 2, the perioperative workflow system backend 102 may access one or more external systems and databases 108. This access may include access via intake mechanism 122 that may access external systems in order to obtain data therefrom and may access via output application(s) 124 in order to provide information (e.g., perioperative workflow information) to the external systems and databases 108. Data evaluation application(s) 126 may evaluate data (e.g., obtained from external systems and databases 108 and/or in the back-end's perioperative workflow system database(s) 112 in order to determine information therefrom. The data evaluation application(s) 126 may include: one or more applications to determine consistency of perioperative workflows, etc.

Various applications 110 in the perioperative workflow system backend 102 may be accessible via interface(s) 138. These interfaces 138 may be provided in the form of APIs or the like, made accessible to external users 104 via one or more gateways and interfaces 140. For example, the perioperative workflow application(s) 120 may provide APIs thereto (interface(s) 138), and the backend may provide external access to aspects of the perioperative workflow application(s) 128 (to users 104) via appropriate gateways and interfaces 140 (e.g., via a web-based application and/or an application running on a user's device).

Users' Devices

Users (e.g., medical practitioners, etc.) may access the perioperative workflow system backend 102 using computing devices. It should be appreciated that the box labeled 104 in the drawings may refer to a user's computing device. The devices can be any kind of computing device, including mobile devices (e.g., phones, tablets, etc.), computers (e.g., desktops, laptops, etc.), and the like. Computing devices are described in greater detail below. As noted, each user may have more than one device and may access the system via multiple devices. For example, a nurse may have a desktop computer at a workstation and also a mobile phone and a tablet, and may access the system 102 via any or all of these devices.

FIG. 3A shows aspects of a typical device 300, including device/client applications 302 interacting with device/client storage 304. Device/client storage 304 may include system/administrative data 306, perioperative workflow data 308, and miscellaneous/auxiliary data 310. The device/client application(s) 114 may include system/administrative applications 312, user interface (UI) applications 314, storage applications 316, perioperative workflow applications 318, and other miscellaneous/auxiliary applications 320. The categorization of data in storage 304 is made for the purposes of aiding this description, and those of ordinary skill in the art will realize and appreciate, upon reading this description, that different and/or other categorizations of the data may be used. As should also be appreciated, any particular data may be categorized in more than one way. Similarly, it should be appreciated that different and/or other categorizations of the device/client applications 302 may be used and furthermore, that any particular application may be categorized in more than one way.

Some or all of the components that make up a device may be integrated into a single physical device or appliance (e.g., a laptop computer), or they may all be separate components (e.g., a desktop computer). The connections between some or all of the components may be wireless. As another example, a device may be integrated into a television, a set-top box, or the like. Preferably each user's device has access to (or has built in) a camera or the like.

FIGS. 3B-3D show examples of devices 300-1, 300-2, and 300-3 that may be used within the system 100. These may correspond, e.g., to devices used by the users 104 in FIG. 1. Device 300-1 (FIG. 3B) has an integrated display and input mechanism in the form of touch screen 322. The device 300-1 is integrated into a single component, e.g., a smartphone, a tablet computer, or the like. Device 300-2 (FIG. 3C) is also integrated into a single component, but, in addition to a screen 324, it includes a keyboard 326 and an integrated mouse 328. The keyboard may be a hardware keyboard (e.g., as in the case of a BlackBerry phone). The screen 324 may be a touch screen and the keyboard may be implemented as a software (or virtual) keyboard. Device 300-3 (FIG. 3D) comprises multiple components, including a computer 330, a computer monitor 332, and input/interaction mechanism(s) 334, such as, e.g., a keyboard 336 and/or a mouse 338. The device 300-3 may also include gesture recognition mechanism 340 and one or more sensors 342. The sensors 342 may include microphones, cameras and the like. In addition, the sensors 342 may include specialized sensors for measurement of environmental factors such as radon, gas, electromagnetic radiation and the like. Some or all of these components may be integrated into a single physical device or appliance (e.g., a laptop computer), or they may all be separate components (e.g., a desktop computer). Although the various components of device 300-3 are shown connected by lines in the drawing, it should be appreciated the connection between some or all of the components may be wireless. For example, one or more of the sensors 342 may be wirelessly connected to the device.

Some of the sensors may be incorporated into wearable devices (e.g., Google glass-type systems) possibly with voice recognition.

As another example, a device may be integrated into a television, a set-top box, or the like. Thus, e.g., with reference again to FIG. 3D, the display 332 may be a television monitor and the computer 910 may be integrated fully or partially into the monitor. In this example, the input/interaction mechanisms 334 (e.g., keyboard 336 and mouse 338) may be separate components connecting to the computer 330 via wired and/or wireless communication (e.g., via Bluetooth or the like). In some cases, the input/interaction mechanisms 334 may be fully or partially integrated into a remote control device or the like. These input/interaction mechanisms 334 may use virtual keyboards generated by the computer 330 on the display 332.

These exemplary devices are shown here to aid in this description, and are not intended to limit the scope of the system in any way. Other devices may be used and are contemplated herein.

A User Interface

A user interface (UI) 314 may be implemented, at least in part, on a device 300, and preferably uses the device's display(s) and input/interaction mechanism(s). Use of a UI may require selection of items, navigation between views, and input of information. It should be appreciated that different devices support different techniques for presentation of and user interaction with the UI. For example, a device with an integrated touch screen (e.g., device 300-1 as shown in FIG. 3B) may display UI information on the touch screen 332, and accept user input (for navigation, selection, input, etc.) using the touch screen (perhaps with a software/virtual keyboard for some types of input). A device with an integrated screen, keyboard, and mouse (e.g., device 300-2 as shown in FIG. 3C) may display UI information on the screen 324, and accept user input using the hardware keyboard 326 and hardware mouse 328. If the screen/display 324 is also a touch screen display, then user interactions with the UI may use the screen (e.g., with a virtual keyboard) instead of or in addition to the keyboard 326 and mouse 328. A device with separate components (e.g., device 300-3 of FIG. 3D) may display UI information on the display 332 and accept user input to the UI using the keyboard 336, mouse 338 (and possibly via gesture mechanism 340).

UI Interactions

A UI presents information to a user, preferably in the form of text and/or graphics (including drawings, pictures, icons, photographs, etc.) on the display(s) of the user's device(s). The user may interact with the UI by variously selecting regions of the UI (e.g., corresponding to certain desired choices or functionality), by inputting information via the UI (e.g., entering text, pictures, etc.), and performing acts (e.g., with the mouse or keyboard) to affect movement within the UI (e.g., navigation within and among different views offered by the UI).

The UI application(s) 314 (FIG. 3A) preferably determines (or knows) the type and capability of the device on which it is running, and the UI may vary its presentation of views depending on the device. For example, the UI presented on a touch screen display on a smartphone may have the same functionality as the UI presented on the display of general-purpose desktop or laptop computer, but the navigation choices and other information may be presented differently.

It should be appreciated that, depending on the device, the UI may not actually display information corresponding to navigation, and may rely on parts of the screen and/or gestures to provide navigation support. For example, different areas of a screen may be allocated for various functions, and the UI may not actually display information about these regions or their potential functionality.

Thus, the manner in which UI interactions take place will depend on the type of device and interface mechanisms it provides.

As used herein, in the context of a UI, the term “select” (or “selecting”) refers to the act of a user selecting an item or region of a UI view displayed on a display/screen of the user's device. The user may use whatever mechanism(s) the device provides to position the cursor appropriately and to make the desired selection. For example, a touch screen 332 on device 300-1 may be used for both positioning and selection, whereas device 300-3 may require the mouse 328 (and/or keyboard 336) to position a cursor on the display 332 and then to select an item or region on that display. In the case of a touch screen display, selection may be made by, e.g., tapping or touching the display in an appropriate region. In the case of a device such as device 300-3, selection may be made using a mouse click or the like.

Touch-screen devices (e.g., an Apple iPad, iPhone, etc.) may recognize and support various kinds of touch interactions, including gestures, such as touching, pinching, tapping, and swiping. These gestures may be used to move within and among views of a UI.

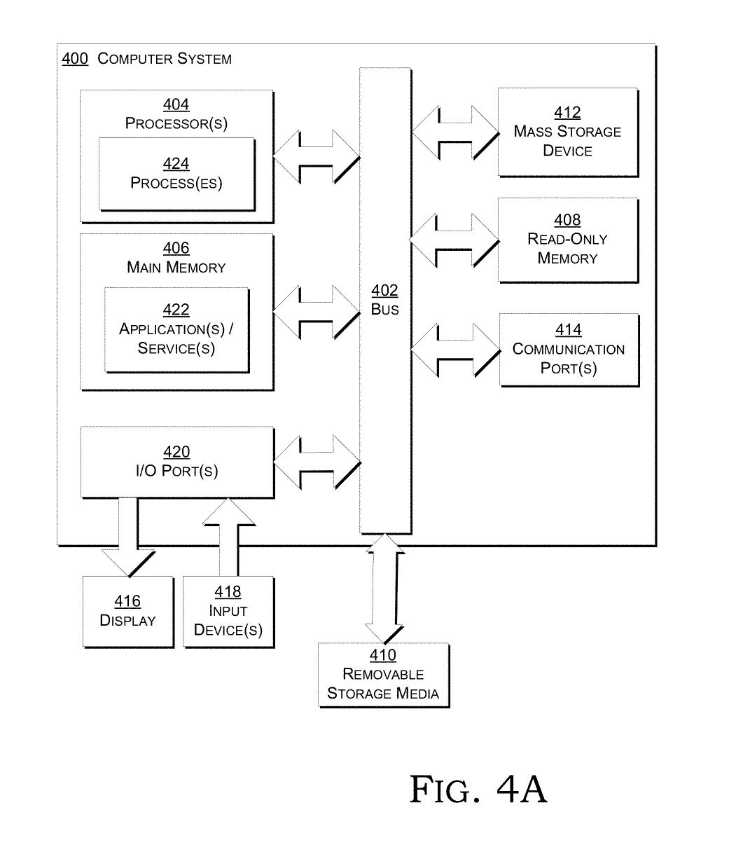

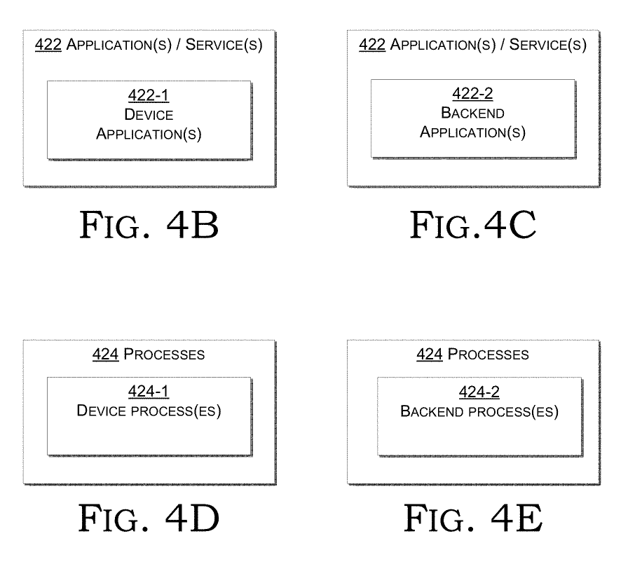

FIG. 4A is a schematic diagram of an exemplary computer system 400 upon which embodiments of the present disclosure may be implemented and carried out. The computer system 400 is discussed in greater detail below.

### Exemplary Implementation & Operation

Clients (users' devices) 104 interact with the perioperative workflow system 100 via an appropriate interface 140 to the perioperative workflow system backend 102. These interactions preferably take place using a user interface (UI) application 314 running on each client.

Overview—Operation

In operation, the framework 100 for perioperative workflow provides a real-time, category agnostic, modular logistics platform with integrated native mobile apps (e.g., iOS & Android) that modernizes hospital workflows, beginning with the all-important perioperative flow.

The perioperative workflow system 102 focuses on the coordination and implementation of a collaborative sequence of steps that make up and determine the efficiency of the perioperative process itself. As should be appreciated, some steps in a workflow may occur in parallel. For example, from while the patient is in surgery, aspects of post-surgery may be prepared. Similarly, from any particular party's perspective, the steps in their role or function may occur in parallel with the steps of other parties. For example, the steps for an OR nurse occur, at least in part, in parallel with those of a surgeon and an anesthesiologist.

The workflow application 120 optimizes the progression of the perioperative flow, from the time a patient is scheduled for surgery or admitted to the hospital to the time they arrive in post-op recovery. The applications track all of the synchronous and asynchronous steps required in the sequence. The scheduling application 118 and workflow application 120 monitor patient flow through the system and remain flexible during the flow in order to adjust to the real-time variability of each step, including the inevitable improvisation that occurs throughout most surgical procedures. Generally, for all users, including administrators, the system provides a real-time view into every critical step within the flow.

The application intuitively and accurately reflects the variety and specificity of each stakeholder's responsibilities.

Equally important is recognizing that the demographics of the user base will vary widely. The app must be easy to use, independent of the user's age, background, or level of technical expertise.

As noted above, each user's device has at least one user interface (UI) (e.g., UI 314 in FIG. 3A), and users access the workflow system 100 via these UIs. The type, role, and sophistication of users differ for different users, as does the information they are expected view and/or input to the system.

The system seamlessly adapts its interface based on the role and permissions associated with each logged-in user. In this way, each user is provided with an interface that presents them with options appropriate for that user. The app's ability to provide a custom view limits the options, such that preferably only the most relevant, timely information and appropriate corresponding actions are presented. Thus, throughout the flow, the app delivers targeted, timely, action-oriented messaging and provides personalized views and insights for all stakeholders (nurses, anesthesiologists, EVS, surgeons, administrators, transport, technicians, etc.), based on the events unfolding in the real-time sequence of the perioperative flow. Up-to-the-minute transparency is preferably provided for every step throughout the perioperative flow, so that delays are avoided, bottlenecks are anticipated, and, ultimately, the workflow keeps moving, thereby making more efficient use of resources and increasing OR throughput.

Data collected from app usage may be used to provide key insights and actionable reports, both in real-time and in digest form, e.g., based on discrete time periods, that can be leveraged to inform such areas as optimal scheduling times based on procedure, ideal pairing of surgeon and anesthesiologist, optimal number of staff, etc. Data collected by the apps may be analyzed by data evaluation mechanism 126 using known learning and analysis techniques. Over time, the system 100 may learn from the data it ingests and aggregates. This learning may be used to provide intelligent guidance and forecasts for the expected timing of various steps in the perioperative workflow, such as the average time needed when a particular surgeon performs a certain procedure on a patient with specific attributes, or the expected time needed for an anesthesiologist to wake up the patient.

The system 100 records pertinent and granular data in real-time throughout every step of the perioperative workflow, including all requests made to technicians, post-op, blood bank, imaging, EVS, and transport. Automated and ad hoc reports generated from the recorded perioperative workflow data include discrete reporting on specific steps or actions (actual surgery start time vs. patient in/out, OR turnover time, anesthesia ready time, etc.) and can be broken down by time, location, personnel, department, and procedure type. In addition, the system 100 analyzes collected data to highlight relevant performance metrics (e.g., top/bottom performers, workflow bottlenecks, and block time utilization). Over time, the system 100 leverages aggregate data on such items as surgeon-specific operative times and patient comorbidities, in order to provide actionable insights (e.g., optimal OR scheduling and shift staffing) and real-time predictive guidance on timing of all OR events and requests.

Implementations of the system 100 are intended fully HIPAA compliant and do not require any integration with existing hospital systems (other than accessing the facility's wireless network). This approach allows an institution to be up and running quickly when it adopts the system 100.

That said, if desired, the logistics platform 100 will seamlessly integrate with existing hospital software (e.g., Epic, Cerna, MEDITECH, etc.) or third party apps and platforms, in order to ingest patient information automatically, scheduling updates, communication protocols, etc., thereby automatically centralizing pertinent data and further expediting the perioperative flow.

Example GUI

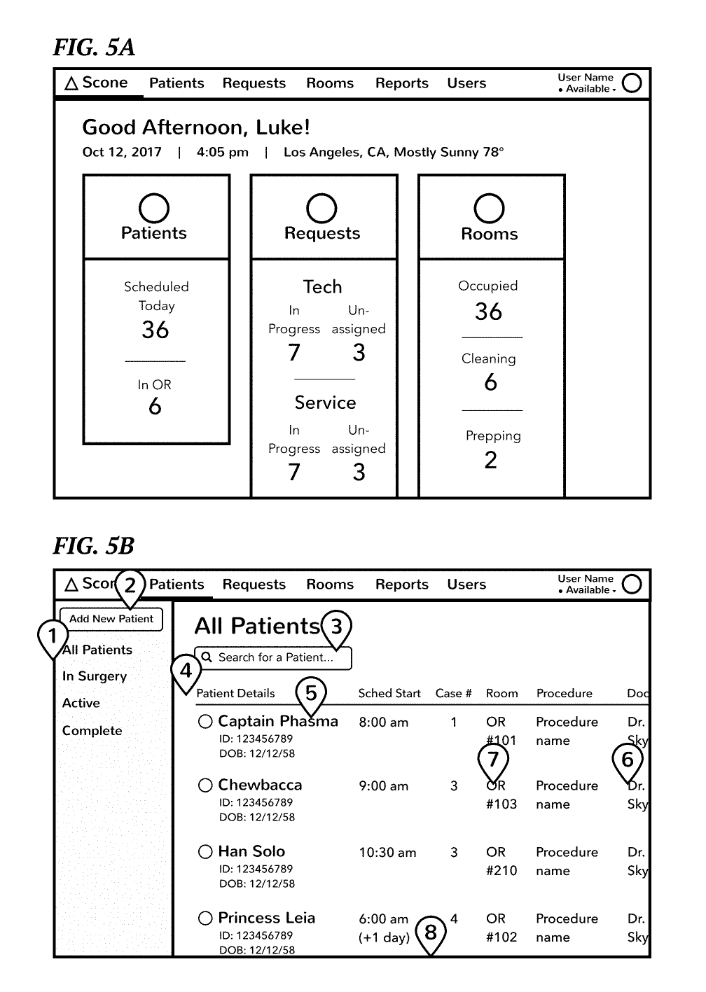

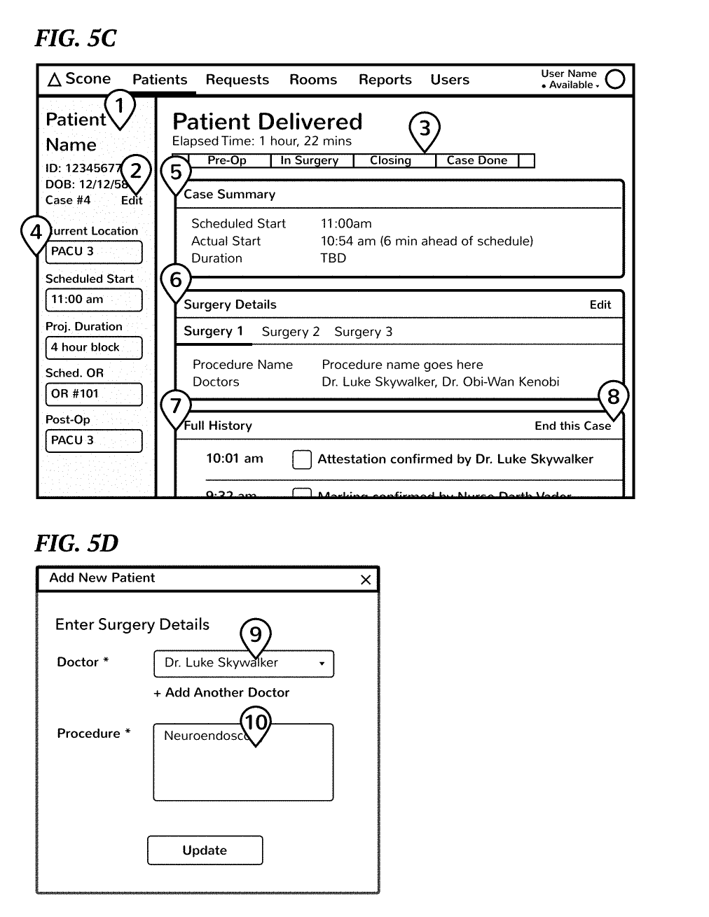

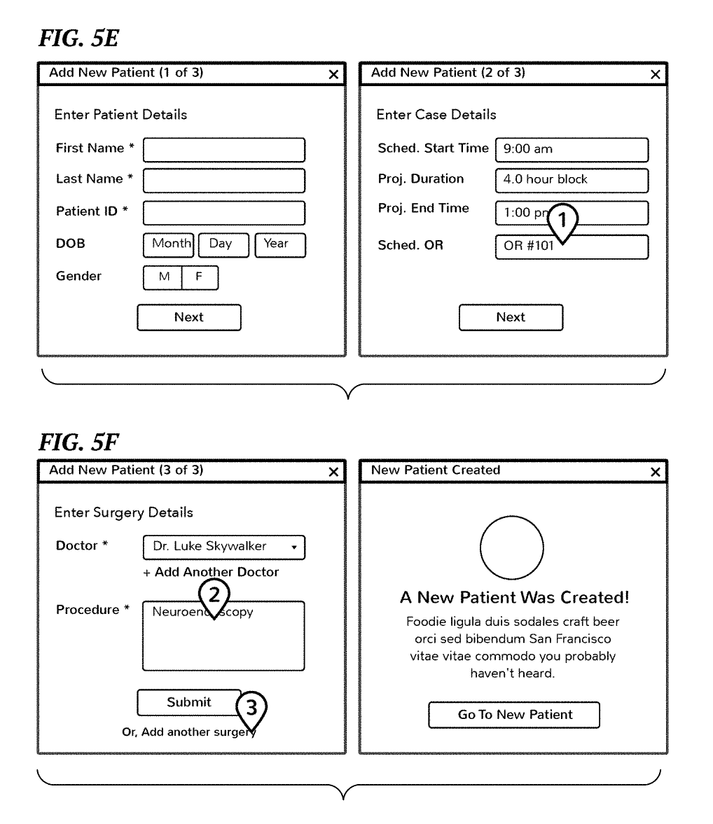

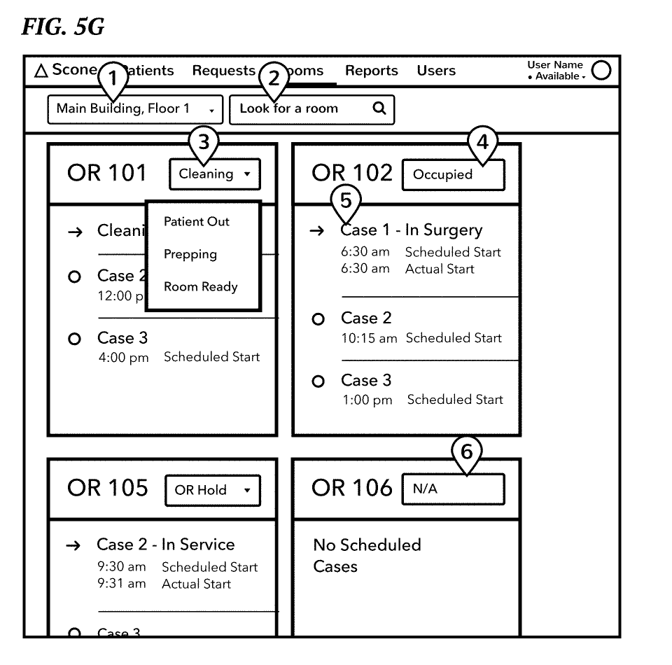

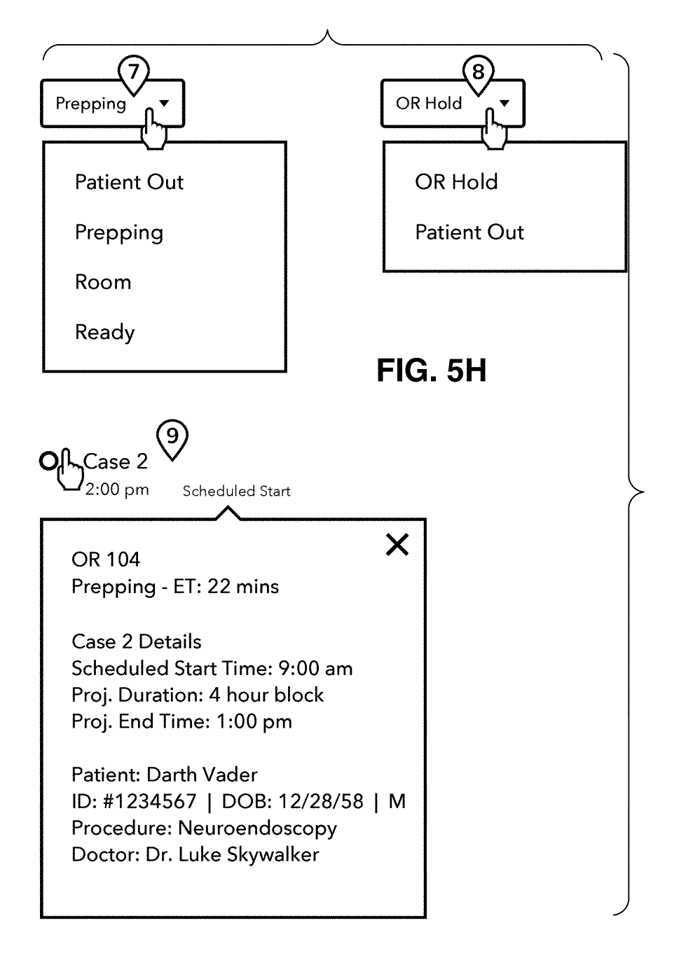

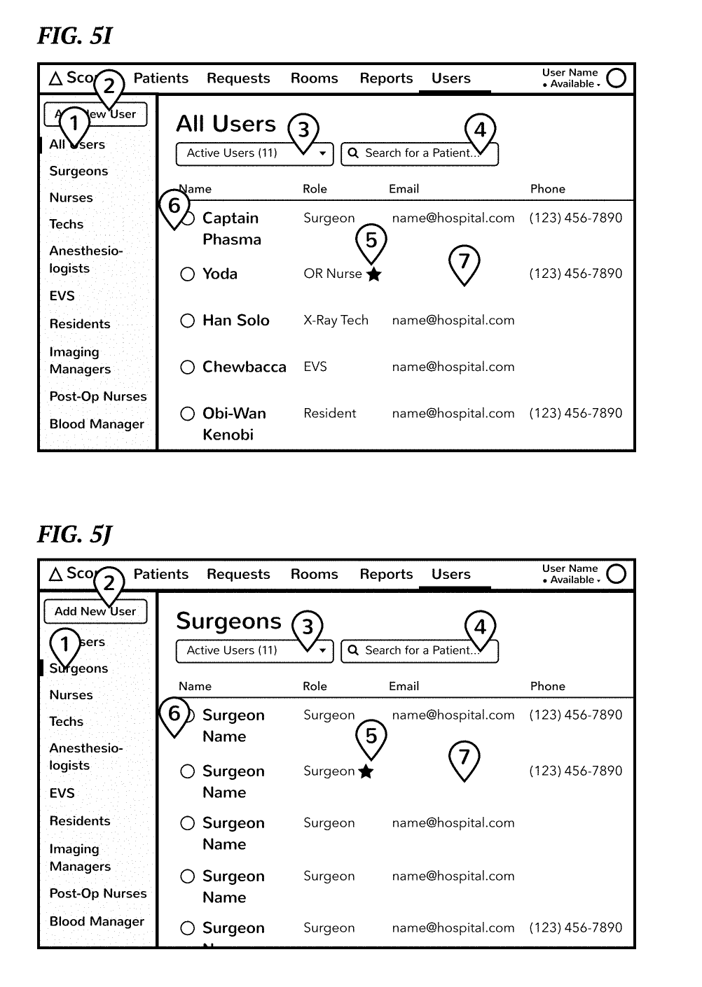

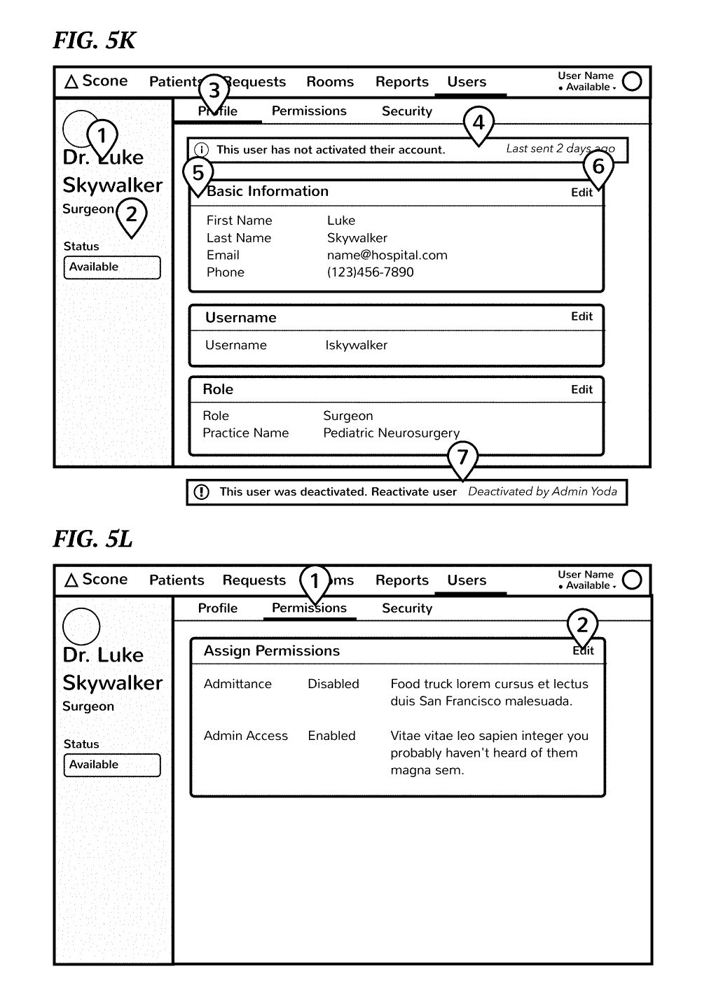

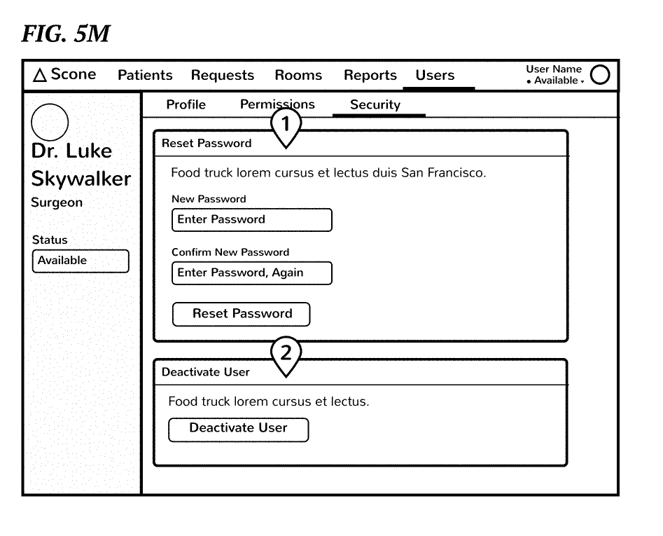

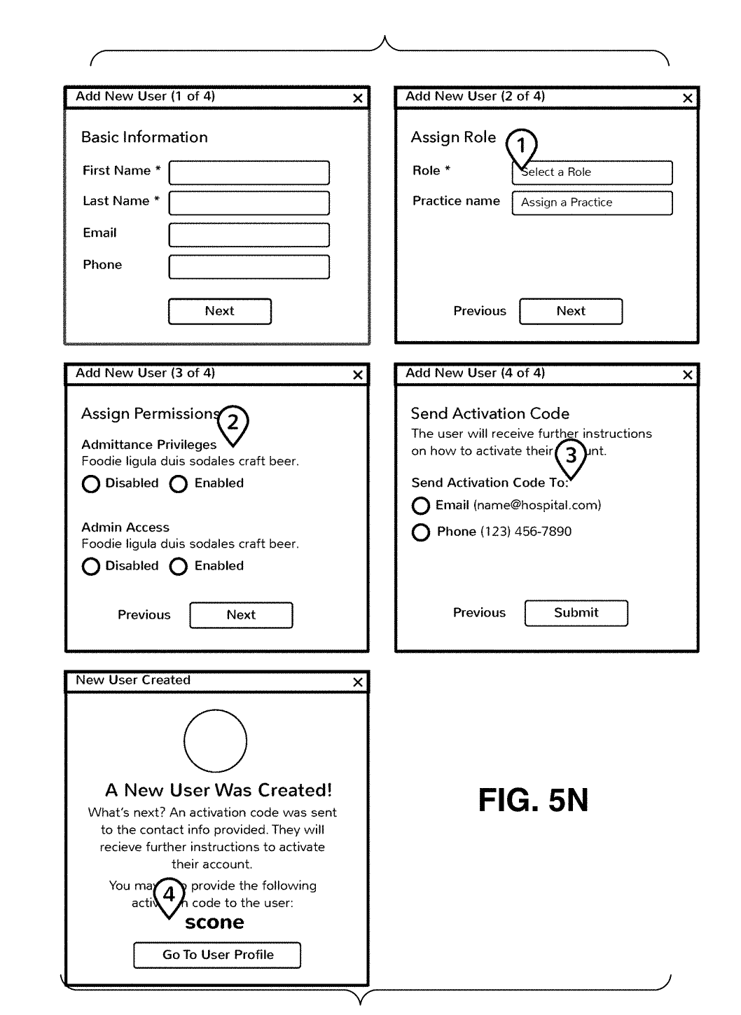

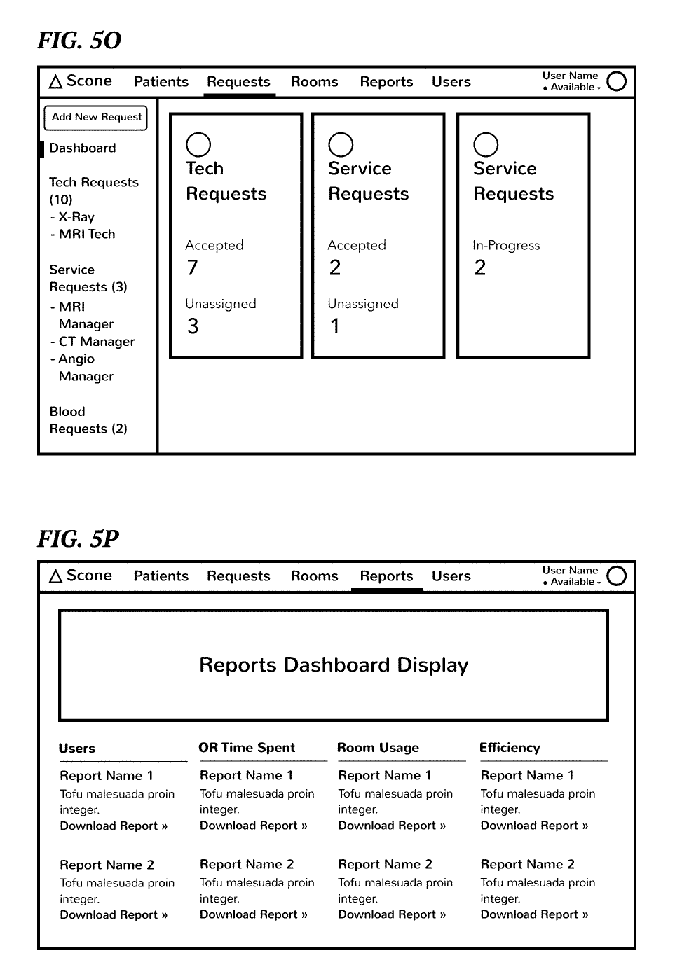

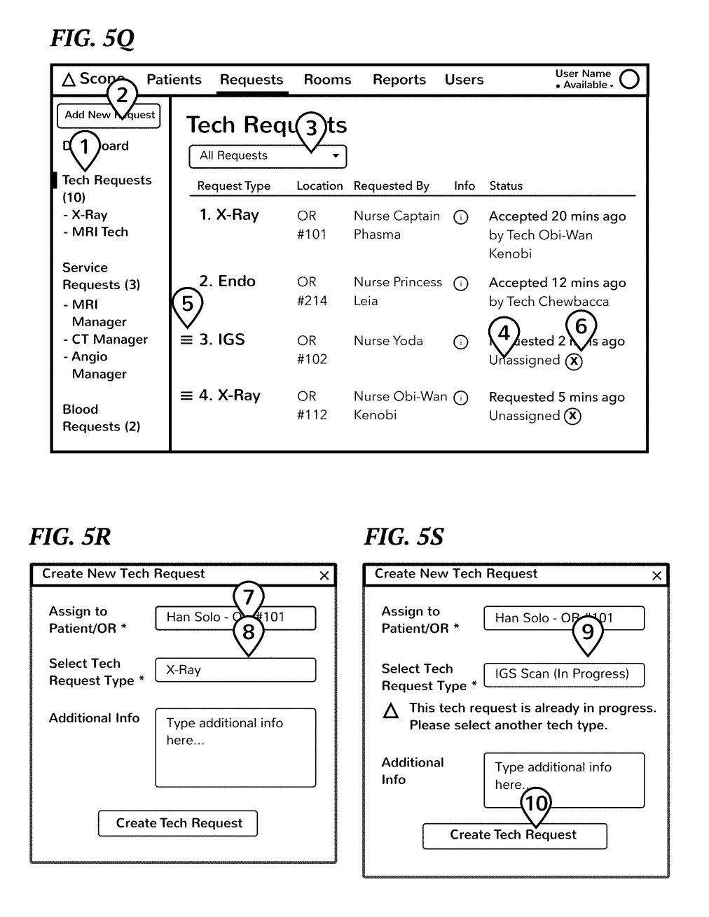

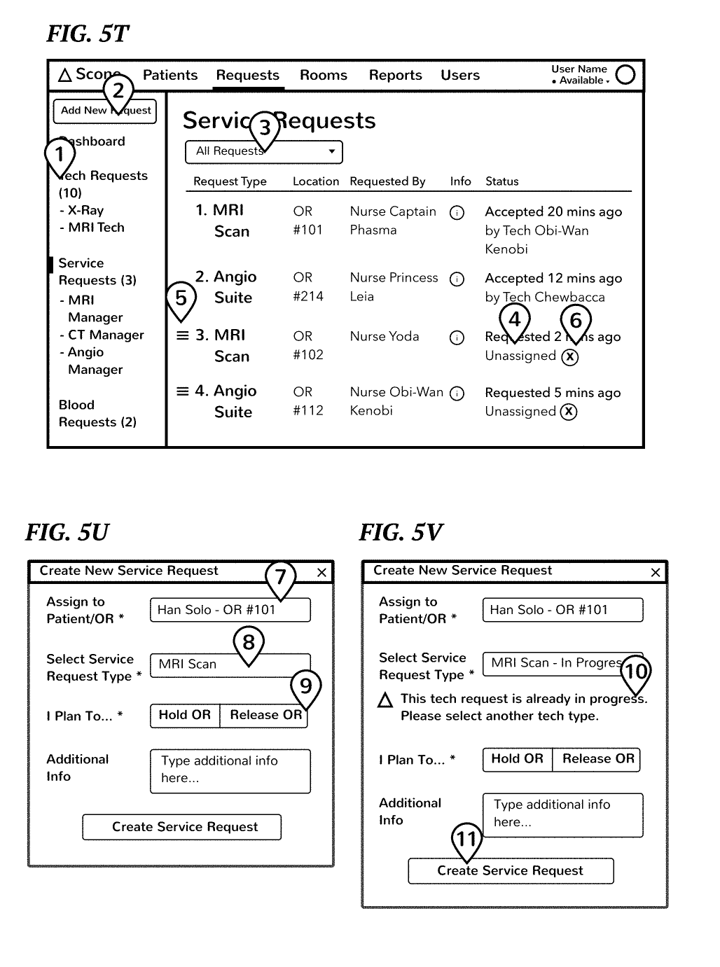

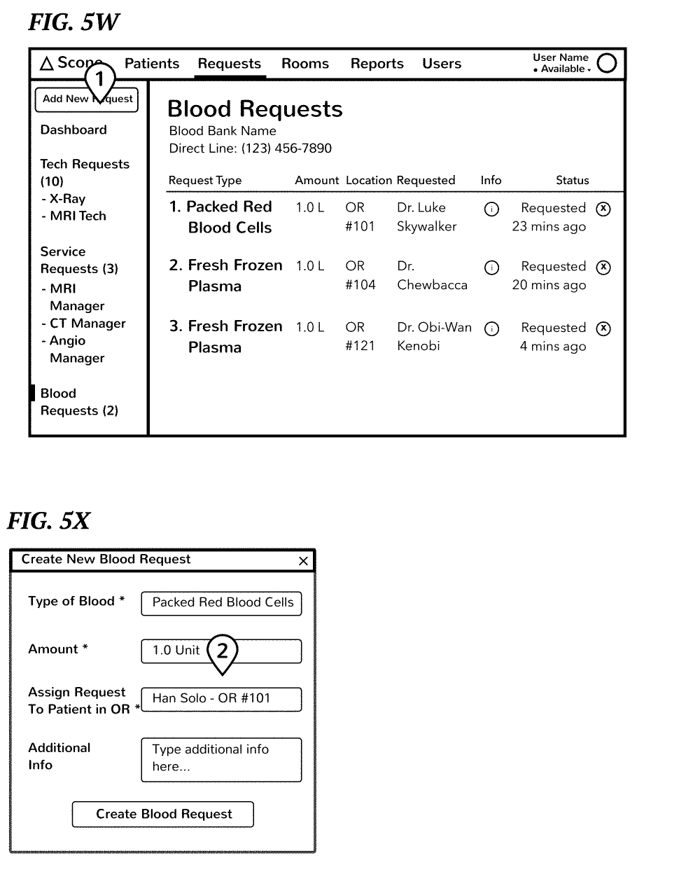

FIGS. 5A-5X, 6A-6H, 7A-7T, 8A-8V, 9A-9X, 10A-10P, 11A-11G, 12A-12I are screen shots of aspects of an exemplary graphical user interface to a perioperative workflow framework in accordance with embodiments hereof. It should be understood that these example screens would appear, at least in part, on the display of a user's device. In some of the examples a series of screens are shown as one continuous display, it being appreciated that a user may need to scroll on the device to see different aspects of the display. Thus, as should be appreciated, some of the example screens shown here may not fit on a single display of a device, and a user may have to move aspects of the screen into the visible display window of their device.

Login/Activation

All users are registered with the system and must login to access/use the system. As part of a user's initial activation, they are given one or more roles. The UI application 314 on the user's device 300 will present the user with a role-appropriate interface. If a user has multiple roles, then the UI is presented, depending on the user's current role.

A user's activation is preferably set up by a hospital super administrator. The administrator enters some or all of the following information:
User's name
User's email
Hospital information
User's Role(s)
User's Practice/Unit
Information about user's device(s)
Other information (e.g., user's phone)

Administration Web Interface

FIGS. 5A-5W depict aspects of an exemplary administration web interface according to exemplary embodiments hereof. The system requires every user to login and the administration web interface provides a login screen. FIG. 5A depicts aspects of an exemplary home page of the administration web interface. FIG. 5B depicts aspects of a screen showing all patients in the administration web interface. 1. The user may filter the patient list in the left side panel by various groups, including: “All Patients” (default), “In surgery,” “Active,” and “Complete.” 2. A new patient may be added to the system. 3. Typing a name into the search box will filter the list below. 4. The default ordering for the list will be alphabetical A-Z, though other orderings may be selected. 5. Clicking on the patient name will go to the patient detail page. 6. Clicking a doctor name will go to the user detail page. 7. Clicking on a room will go to the room detail page. 8. List view will scroll infinitely as needed.

FIG. 5C depict aspects of a patient detail page in the administration web interface. 1. Patient detail area shows: name, ID, DOB, and Gender info; 2. Select edit link to edit patient details; 3. Current status chart; 4. Patient detail area. These fields may be editable by an administrator: Current location, Scheduled Time, Projected Duration, Scheduled OR, and Post-Op; 5. Case summary: Show the following info (if known): Scheduled start, Actual start, and duration; 6. Surgery details with link to edit details. Multiple surgeries (if applicable) may be toggled by tabs (if applicable); 7. Full history view of patient; 8. End Surgery option allows administrator to end the surgery if needed—double confirmation required. The doctor may be updated (e.g., using the edit surgery details edit link), or the administrator may add another doctor. The administrator may update the procedure name.

FIGS. 5D-5E depict aspects of an interface to add a patient in the administration web interface. 1. Available ORs will be listed in the dropdown menu. 2. A procedure is entered via the open text field. 3. If multiple surgeries are required for the patient, an administrator may click on the link to add another surgery. After the new patient has been added, a dialog box or tab confirming the new patient may be displayed.

FIGS. 5F-5G depict aspects of a rooms interface in the administration web interface. 1. Filter rooms via dropdown. 2. Typing a room into the search box will filter the list below. 3. If a room status is editable, a dropdown will appear in the room header. 4. A room status may not be changed if it is occupied. 5. The current case for each room is always listed at the top. Scheduled start times for cases will be listed if known. Actual start time will be listed if known. 6. “N/A” status will appear if there are no scheduled cases for a room. 7. If a room status is editable, a dropdown will appear in the room header. 8. For an OR Hold room status, the admin may update the status to “Patient Out.” 9. Clicking on a case row will surface a pop-up window with more case details.

FIG. 5H depicts aspects of an “all users” interface in the administration web interface. 1. The user may filter the patient list in the left side panel by All Users (default), and by specific role. 2. A new user may be added to the system. 3. The list view may be sub-filtered by: Active Users, Not Activated, and Deactivated. 4. Typing a name into the search box will filter the list below. 5. Users with admin rights will be assigned a badge next to their role. 6. The default ordering for the list will be alphabetical A-Z, other orderings may be selected. 7. Data fields will be empty if the user does not provide the contact info during onboarding.

FIG. 5I depicts aspects of a doctors list interface in the administration web interface. 1. The user may filter the patient list in the left side panel by All Users (default), and by specific role. 2. A new user may be added to the system. 3. The list view may be sub-filtered by: Active Users, Not Activated, and Deactivated. 4. Typing a name into the search box will filter the list below. 5. Users with admin rights will be assigned a badge next to their role. 6. The default ordering for the list will be alphabetical, with other orderings selectively available. 7. Data fields will be empty if the user does not provide the contact info during onboarding.

FIG. 5J depicts aspects of a user detail page in the administration web interface. 1. User detail area shows: User avatar, Name, and Role. 2. Editable status dropdown—update requires double confirmation. A notification confirming the status was updated will also be sent to the user. 3. Profile tabs: Profile (default), Permissions, and Security. 4. Information banner example (if applicable): User has been added to the system but has not yet activated their account. 5. Profile tab shows the following modules: Basic info, username, and role 6. Link to edit info appears in the top right corner of each module. 7. Information banner additional example: User has been deactivated.

FIG. 5K depicts aspects of a user permissions page in the administration web interface. 1. Select the permissions tab to toggle to the permissions area. 2. Select the edit link to edit permissions for this user.

FIG. 5L depicts aspects of a user security page in the administration web interface. 1. Reset Password for the user by entering a new password in the fields. 2. Deactivate this user. Requires double confirmation.

FIG. 5M depicts aspects of adding a new user in the administration web interface. 1. A secondary dropdown appears after a role is selected. The dropdown may change depending on what role is selected. 2. Assign permissions to the user (if applicable). 3. Select the contact method where the activation code should be sent (email, phone, or both). 4. The activation code will appear on the confirmation screen (in addition to being sent to the user).

FIG. 5N depicts aspects of request dashboard in the administration web interface.

FIG. 5O depicts aspects of a reports interface in the administration web interface.

FIGS. 5P-5R depict aspects of a tech request interface in the administration web interface. 1. The user may filter tech requests by available type from the links in the left panel. 2. A new request may be added to the queue. 3. Requests may be filtered via dropdown to: In-progress, or Unassigned. 4. Unassigned requests will be highlighted. 5. Unassigned requests may be re-ordered via drag drop interaction. 6. Only Unassigned requests may be cancelled. 7. Patients with a tech-request able state (i.e., In OR and not case done) will appear in the dropdown. 8. A list of available tech requests will appear in the dropdown. 9. Only 1 tech request of each type is permitted per patient. If a tech type has already been requested, an error message appears underneath the dropdown. 10. If a tech type has already been requested for the patient, the Create Request button will be disabled.

FIGS. 5S-5U depict aspects of a service request interface in the administration web interface. 1. The user may filter service requests by available type from the links in the left panel. 2. A new request may be added to the queue. 3. Requests may be filtered via dropdown to: In-progress, or Unassigned. 4. Unassigned requests will be highlighted. 5. Unassigned requests may be re-ordered via drag drop interaction. 6. Only Unassigned requests may be cancelled. 7. Patients with a service-requestable state (i.e., In OR and not case done) will appear in the dropdown. 8. A list of available service requests will appear in the dropdown. 9. The admin selects a post-service option: Leave and Hold OR/Leave and Release OR. 10. Only one service request is permitted per patient. If a service has already been requested, an error message will surface underneath the dropdown. 11. If a service request was already requested for the patient, the Create Request button will be disabled.

FIGS. 5V-5W depict aspects of a blood request interface in the administration web interface. 1. A new request may be added to the queue. 2. Patients with a blood-requestable state (i.e., In OR and not case done) will appear in the dropdown.

Service Manager Flow

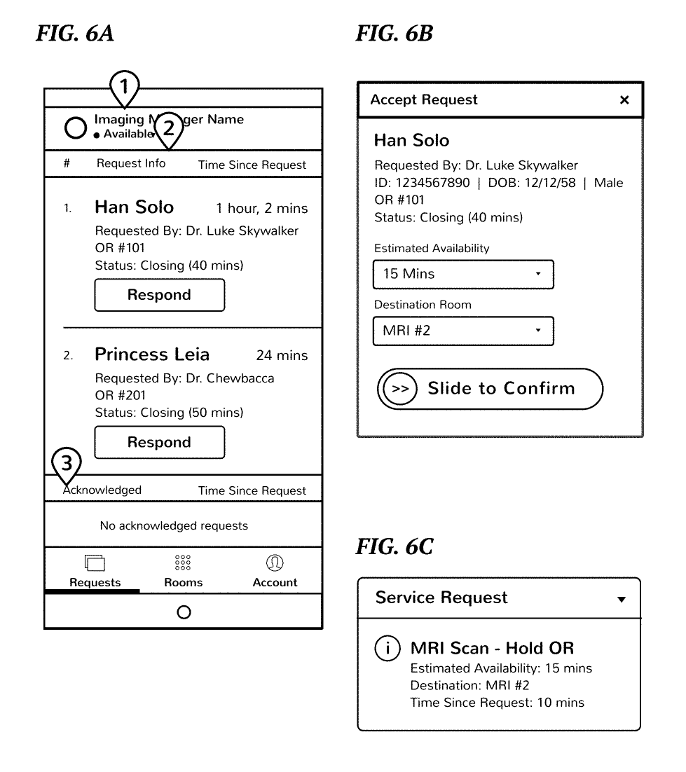

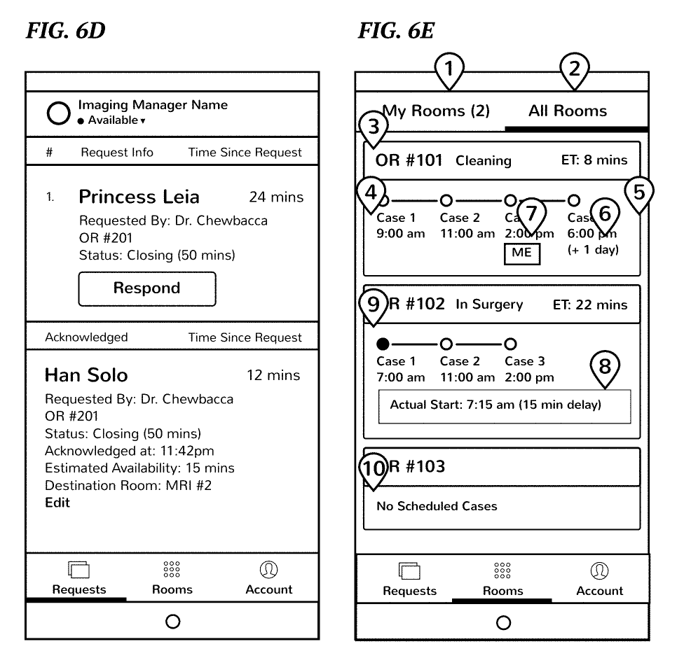

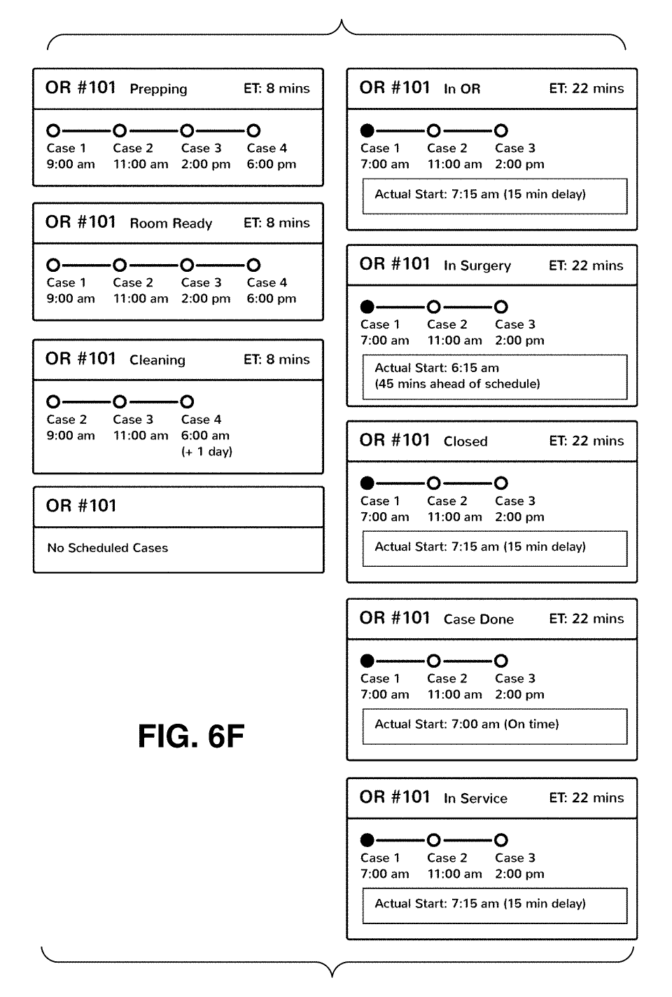

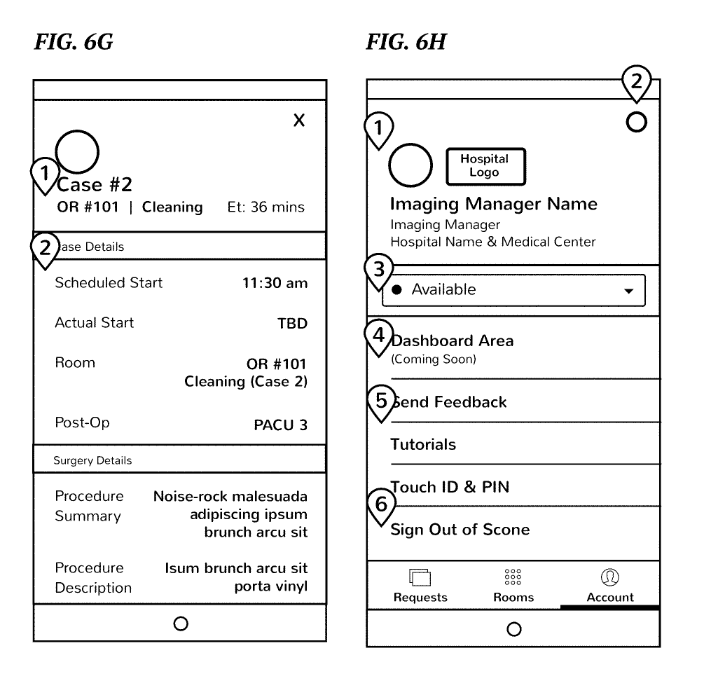

FIGS. 6A-6H depict aspects of a service manager flow according to exemplary embodiments hereof.

Service Manager Flow—Requests

FIGS. 6A-6C depict aspects of a requests tab (in the service manager flow view) according to exemplary embodiments hereof. FIG. 6A shows a service request tab with open requests in a list view. 1. The user may update their status directly from the status dropdown. 2. Open requests are shown in the “requests” area. A “Closing estimate” counts down (and stops at zero minutes, even if it goes longer). The user may choose to respond to any unassigned request in the queue. Requests are preferably ordered in priority based on the time since request. 3. Requests that have been responded to are listed in the acknowledged requests area. If there are no acknowledged requests, the UI shows an empty state message. Preferably, a number count of unassigned requests appears in the navigation bar. Note that if there are no open service requests, the tab may state “No requests. We will notify you when the next action is needed.” or a similar message. If there is no immediate action required by the user, the tab preferably shows a waiting message.

FIG. 6B shows an accept request screen that is displayed when the user selects “Respond” to an open request (e.g., in FIG. 6A). The user may respond, e.g., that they are available in an estimated amount of time or available immediately. The user is presented with a destination room (in this example, “MRI 2”). FIG. 6C Once a response is confirmed, the original requestor is presented with a response message in the Patient details request tab.

FIG. 6D shows the requests tab with an acknowledged request. An acknowledged request will display the following info: Requested by, room, status, acknowledged timestamp, estimated availability, and destination room. The user may update their response by selecting the edit link (e.g., to change destination room).

Service Manager Flow—Rooms

FIGS. 6E-6G depicts aspects of a Rooms tab (in a service manager flow view) according to exemplary embodiments hereof. Room number will determine card order. In some implementations the room status may be one of “Prepping,” “Cleaning,” “Room Ready,” or occupied states: “In OR,” “In Surgery,” “Closed,” “Case Done,” “In Service.”

FIG. 6E In a “My ROOMS: Card View, 1. Room cards with my cases (denoted with a “Me” badge underneath the case info) will appear in the list below. In an “ALL ROOMS: Card View,” 2. Show all room cards in the list below; 3. Room card header shows the following info: OR #, room status, room status elapsed time. The header bar color will be colored according to room status: e.g., orange=cleaning, prepping, red=occupied, green=room ready, gray=no scheduled cases; 4. Case # and scheduled time will appear in the display if available. Selecting the case # will display the room detail view (FIG. 6G); 5. The user may swipe to see more cases if needed; 6. If a displayed case is +X days in advance, the text “(in X days)” will appear. 7. My cases will be highlighted with a “Me” badge underneath the case info. 8. In OCCUPIED States (In OR, In Surgery, Closed, Case Done, In Service, OR Hold): the actual start time will be displayed for occupied rooms. The display next to the actual start will show the amount of delay or time ahead of schedule as applicable (delayed/on time/ahead of schedule); 9. The in-progress case is highlighted in the chart. 10. For NO SCHEDULED CASES: 1. An empty state message will appear if there are no other cases scheduled for the room.

FIG. 6F depicts aspects of room card status examples in various states: “Prepping,” “Cleaning,” “Room Ready,” or occupied states: “In OR,” “In Surgery,” “Closed,” “Case Done,” “In Service.”

FIG. 6G depicts aspects of a Room Details view (in a service manager flow view) according to exemplary embodiments hereof. 1. The room detail header area shows the following info: Case #, room #, room status, room status elapsed time. The header background will be colored according to room status: orange=cleaning, prepping, red=occupied, green=room ready, gray=no scheduled cases; 2. Show the following info (if known): case details, surgery details, patient info (if My Patient).

Service Manager Flow—Account

FIG. 6H depicts aspects of an account tab (in a service manager flow view) according to exemplary embodiments hereof. 1. The account header area shows the following info: user's name, photo/avatar, role, hospital name, and logo. 2. A settings link to update profile and account settings is located in the top right corner of the header area. 3. A dropdown menu allows the user to change their status (Available, Away, Do not disturb) 4. A user dashboard area provides a performance chart (e.g., weekly performance) and other data points. 5. Other links (Send feedback, tutorials, Touch ID & PIN) will take the user to more detailed views on those areas. 6. A link to sign out of the app will be listed at the bottom of the page.

Non-OR Nurse Flow

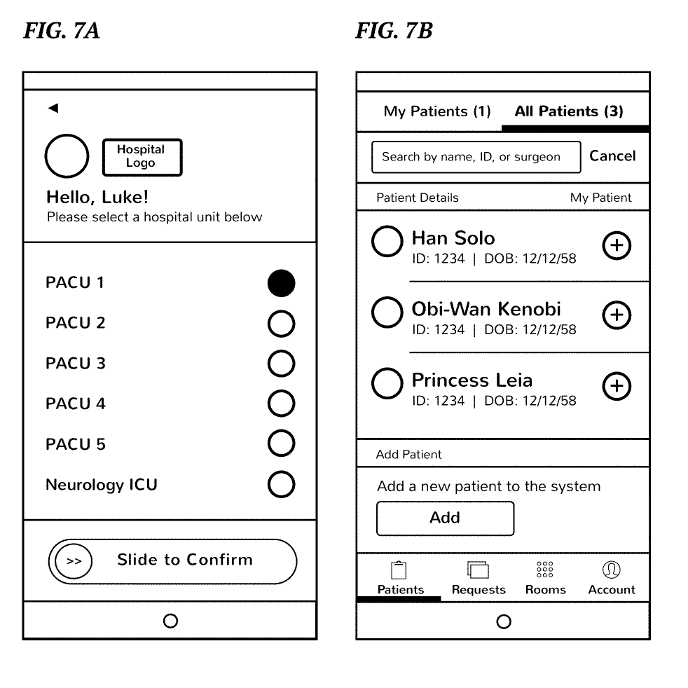

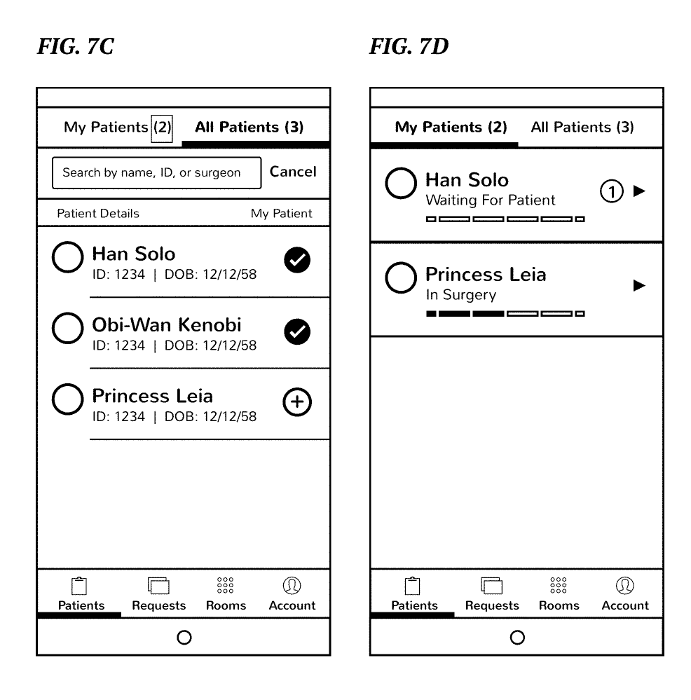

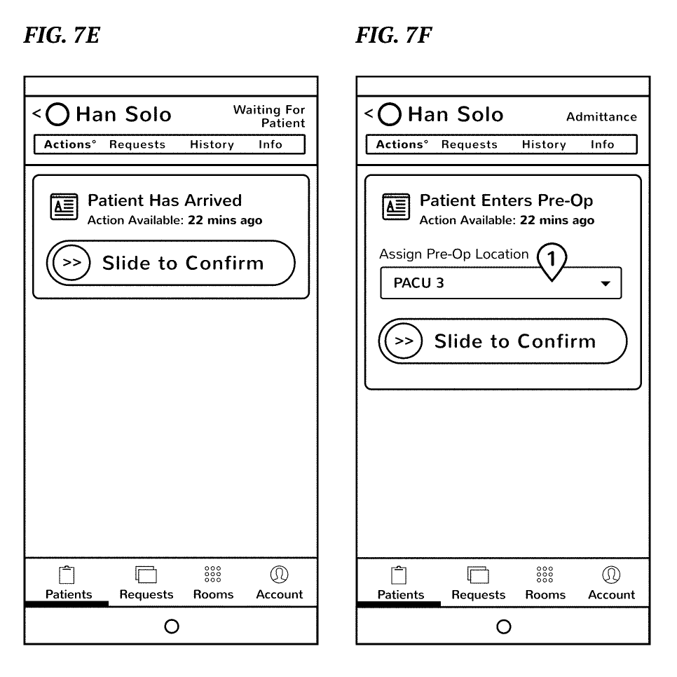

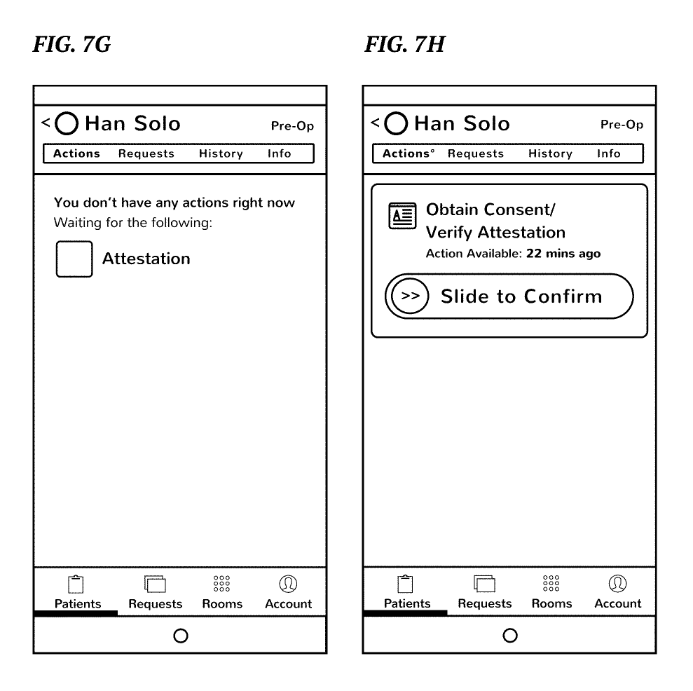

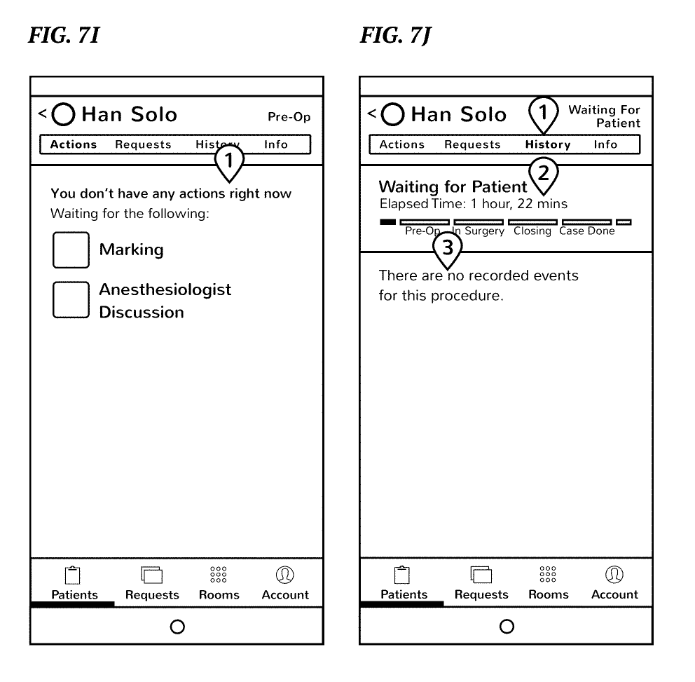

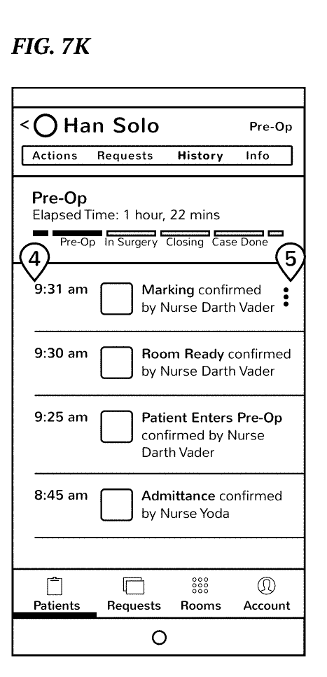

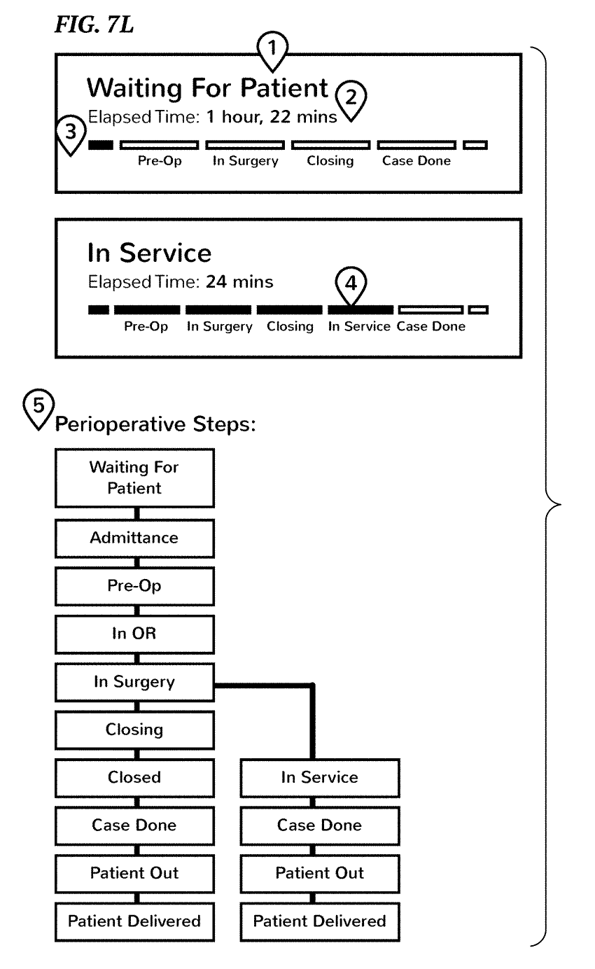

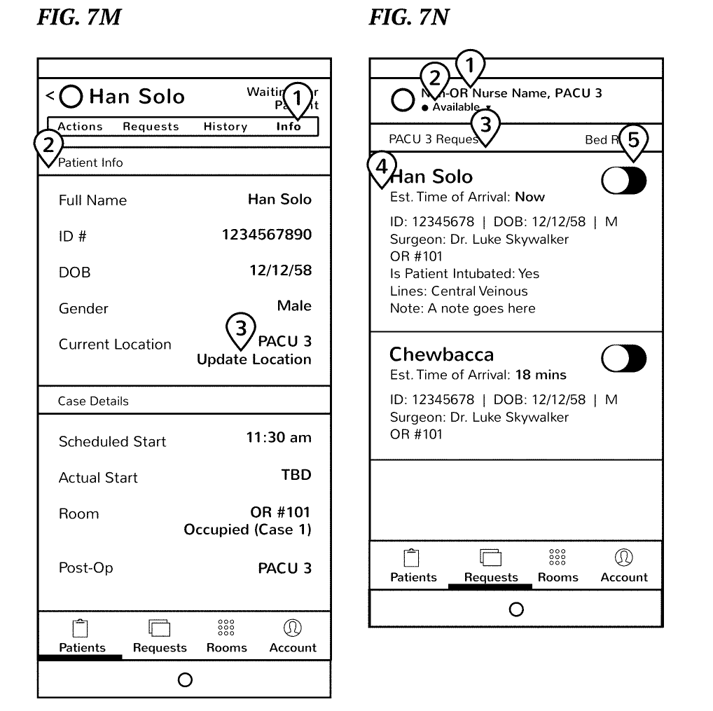

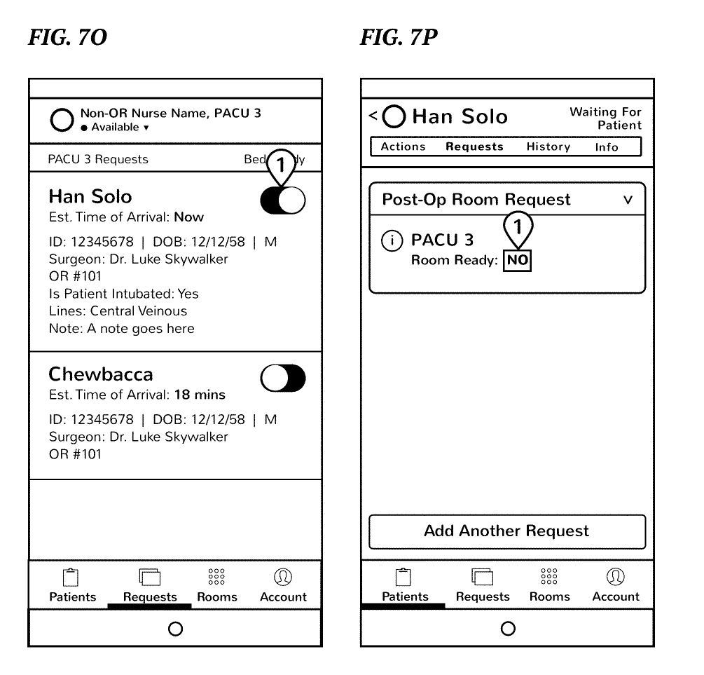

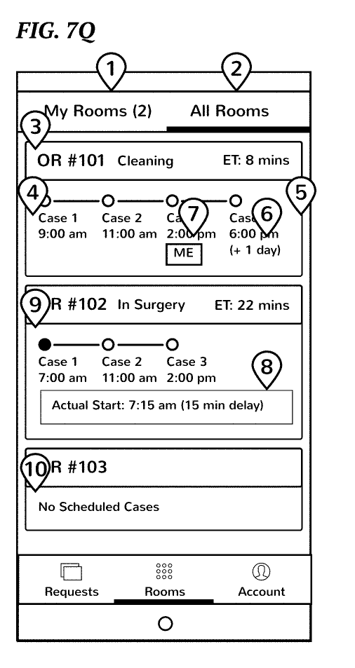

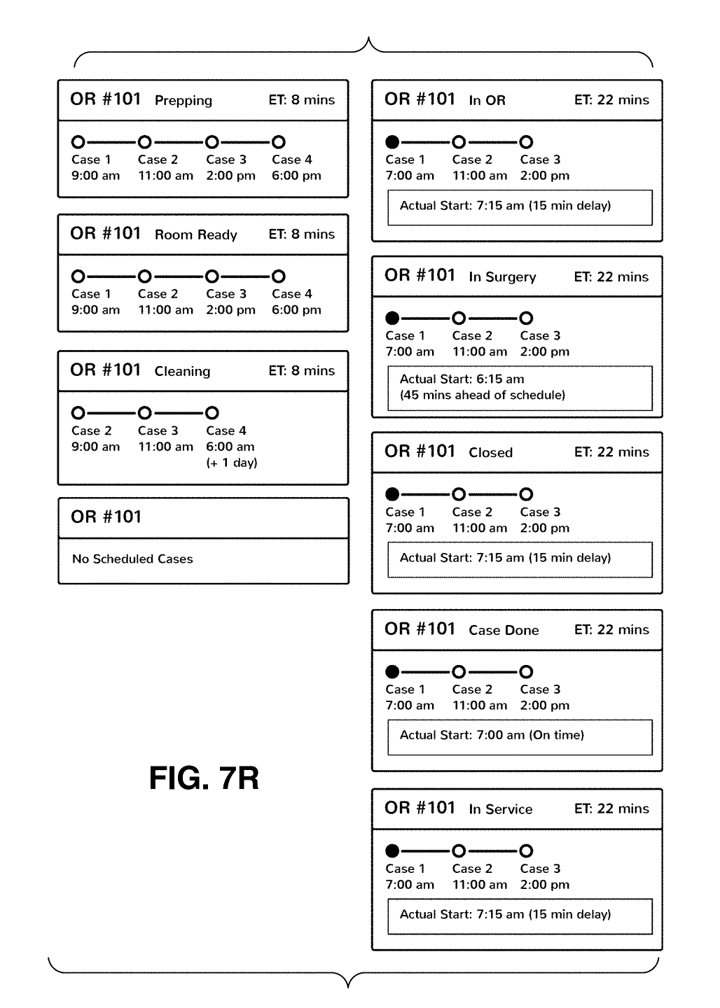

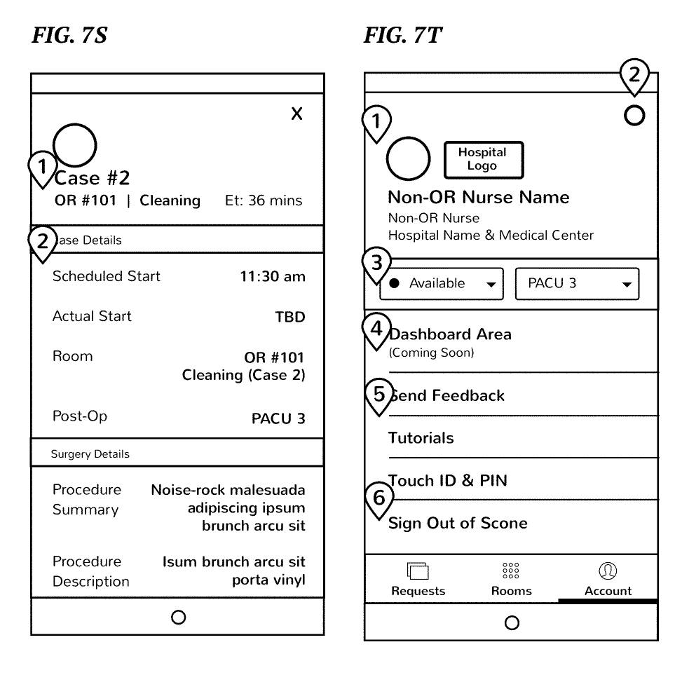

FIGS. 7A-7T depicts aspects of a non-OR nurse flow according to exemplary embodiments hereof.

Non-OR Nurse Flow—Login

The user may be presented with a login tab (in a non-OR nurse flow view) according to exemplary embodiments hereof. The user may enter appropriate user data to login to the device such as User ID and Password. The user may also be asked if the device is a shared device (e.g. via a checkbox) and if the answer is affirmative, the application may automatically log the user out after a preset amount of time (preferably coinciding with the end of the user's shift). The user may be asked to confirm an automatic logout to prevent erroneous logouts. FIG. 7A depicts aspects of a secondary login screen that a user may see if the user is a non-OR nurse associated with multiple units. When presented with this screen the user chooses which unit they are to be working in.

Non-OR Nurse Flow—all Patients

FIGS. 7B-7C depicts aspects of an all patients tab (in a non-OR nurse flow view) according to exemplary embodiments hereof.

With reference to FIG. 7B, the search box allows the user to search all patients by patient name, ID, or doctor/surgeon, with dynamic filtering of the list below. The display shows details of each patient. A nurse may add a patient to the My Patients view akin to “following” a patient and receiving updates. If the user has administrative rights to admit patients, the add new patient option will appear.

With reference to FIG. 7C, selecting the My Patient checkbox in the list will add/remove the patient to My Patient list. In addition, the number counter in the My Patient tab bar will highlight to show if a patient has been added or removed from the list.

Non-OR Nurse—My Patient

FIG. 7D depicts aspects of a My Patient tab (in a non-OR nurse flow view) according to exemplary embodiments hereof.

In the tab depicted in FIG. 7D, patients added to the My Patient list may show the following patient info: Patient name, current status, status bar chart, and gender icon. If an action is available for the patient, a number badge will appear in the row. Selecting anywhere in the row will take the user to the patient detail page where actions on the patient may be performed. Note that if there are no assigned patients, the tab may state “No patients. We will notify you when you have patients in your practice.” or a similar message.

Non-OR Nurse—Patient Details

FIGS. 7E-7I depict aspects of a “Patient Detail” (in a non-OR nurse flow view) according to exemplary embodiments hereof. FIG. 7E depicts aspects of a non-OR nurse's Patient details view, for actions (waiting for patient). The top section of the page displays the following patient details: Patient name, gender icon, patient status, and segment controller with multiple options. The user may select in the segment controller to toggle between: actions (default), requests, history, and info. If an action is available, show an alert dot next to the actions text. The user may also return to the previous patient list view by selecting the back arrow at the top of the screen. In the main actions view, an active Action card shows: Action title/icon, and how long the action has been available. The interface uses a slide button interaction to confirm task. The card will slide away once confirmed.

FIG. 7F depicts aspects of a non-OR nurse's Patient detail view, for actions (admittance). 1. The user will confirm that the Pre-Op location is correct in the action card (location is pre-filled with the Non-OR nurse's logged in unit). The user may update the location by selecting the field and selecting another room via modal.

FIG. 7G depicts aspects of a non-OR nurse's My Patient view, for actions (pre-op waiting for next step). If there is no immediate action by the user, show a waiting message and the action(s) that the user is waiting to be completed. Once completed, the next action will appear.

FIG. 7H depicts aspects of a non-OR nurse's My Patient view, for pre-op (continued) with active steps. If a step is available for the user to take action, the appropriate card will be displayed. Slide button interaction to confirm task. The card will slide away once confirmed.

FIG. 7I depicts aspects of a non-OR nurse's My Patient view, for pre-op (continued) waiting for next step(s). 1. For Non-OR Nurses, there are no following actions to display until the patient leaves the OR. Continue showing the waiting for action/no action message screen until the user selects another patient to view.

Non-OR Nurse—Full History

FIGS. 7J-7L depict aspects of a full history display (in a non-OR nurse flow view) according to exemplary embodiments hereof.

As shown in FIGS. 7J-7K, 1. The full history view is displayed by selecting history in the segment controller. 2. The patient status area shows the following info: Current patient status, elapsed time, and status bar chart with current status highlighted in green. 3. If there are no recorded actions, show an empty state message. 4. Display completed action: icon, completed/confirmed by, timestamp, and any other details as needed (examples of most common actions are shown above but may vary depending on hospital). 5. Modified actions (undo, skipped) may be accessed by selecting the more icon.

FIG. 7L shows: Patient status area during various stages of the perioperative flow. 1. The current patient status (current step in the perioperative flow) is listed. 2. The elapsed time of the current patient status is displayed. 3. The status bar chart displays the current step in the perioperative status in green. 4. During a service request, the status card area updates to display the In Service status title in orange, and the status bar chart expands to include an In Service section. 5. The perioperative flow steps may be customized per hospital.

Non-OR Nurse—Info

FIG. 7M depicts aspects of an info display (in a non-OR nurse flow view) according to exemplary embodiments hereof. 1. The info view is displayed by selecting info in the segment controller. 2. Patient info is grouped by sections: Patient info (Full name, ID, DOB, gender, current location), Case details (Scheduled start, actual start, room, post-op), Surgery details (Procedure(s) name, surgeon(s)), Remove from My Patients. 3. A user may update the current patient location only during Pre-Op and Post-Op, and not during surgery. If the location is editable, the location link will appear in blue.

Non-OR Nurse—Mark Bed Ready (Requests Tab)

FIGS. 7N-7Q depict aspects of a Requests tab (in a non-OR nurse flow view) according to exemplary embodiments hereof.

FIG. 7N shows a Requests tab with open requests. 1. Show user name and currently logged-in unit. 2. Show user's availability status (available/away). 3. Sort list by priority order: —No bed ready, no nurse assigned, —No bed ready, nurse assigned, —Bed ready, no nurse assigned, —Bed ready, nurse assigned. 4. Request details show: Patient name, est. time of arrival (time will count down based, request details (if entered in request form), and bed ready/not ready toggle button. New requests will be highlighted and the number count will appear in the bottom navigation. 5. Selecting the bed ready toggle button will mark the bed as ready/not ready. The toggle button may be selected again to switch the ready/not-ready state. Note that if there are no open requests, a message such as “No Requests. We'll notify you when there are open requests in the queue.” or similar may be displayed. If there is no immediate action by the user, the tab preferably shows a waiting message.

As shown in FIG. 7O, at 1, when a bed is marked as ready, the toggle button will turn green. In addition, the request row will highlight in a subtle green color.

FIG. 7P depicts aspects of the Post-Op Room requester's Patient detail view with the in-progress request card. If the room is not ready (bed not marked ready by the Non-OR Nurse), the request card will show Room Ready=“No.” If the room is ready (bed marked ready by the Non-OR Nurse), then the request card will show Room Ready=“Yes”

Non-OR Nurse Flow—Rooms

FIGS. 7Q-7S depict aspects of a Rooms tab (in a non-OR nurse flow view) according to exemplary embodiments hereof. Room number will determine card order. In some implementations the room status may be one of “Prepping,” “Cleaning,” “Room Ready,” or occupied states: “In OR,” “In Surgery,” “Closed,” “Case Done,” “In Service.”

As shown in FIG. 7Q, in a “My ROOMS: Card View, 1. Room cards with my cases (denoted with a “Me” badge underneath the case info) will appear in the list below. In an “ALL ROOMS: Card View,” 2. Show all room cards in the list below; 3. Room card header shows the following info: OR #, room status, room status elapsed time. The header bar color will be colored according to room status: orange=cleaning, prepping, red=occupied, green=room ready, gray=no scheduled cases; 4. Case # and scheduled time will appear in the display if available. Selecting the case # will display the room detail view (FIG. 7S); 5. The user may swipe to see more cases if needed; 6. If a displayed case is +X days in advance, the text “(in X days)” will appear. 7. My cases will be highlighted with a “Me” badge underneath the case info. 8. In OCCUPIED States (In OR, In Surgery, Closed, Case Done, In Service, OR Hold): the actual start time will be displayed for occupied rooms. The display next to the actual start will show the amount of delay or time ahead of schedule as applicable (delayed/on time/ahead of schedule); 9. The in-progress case is highlighted in the chart. 10. For NO SCHEDULED CASES: 1. An empty state message will appear if there are no other cases scheduled for the room.

FIG. 7R depicts aspects of room card status examples in various states: “Prepping,” “Cleaning,” “Room Ready,” or occupied states: “In OR,” “In Surgery,” “Closed,” “Case Done,” “In Service.”

FIG. 7S depicts aspects of a Room Details view (in a non-OR nurse flow view) according to exemplary embodiments hereof. 1. The room detail header area shows the following info: Case #, room #, room status, room status elapsed time. The header background will be colored according to room status: orange=cleaning, prepping, red=occupied, green=room ready, gray=no scheduled cases; 2. Show the following info (if known): case details, surgery details, patient info (if My Patient)

Non-OR Nurse Flow—Account

FIG. 7T depicts aspects of an account tab (in a non-OR nurse flow view) according to exemplary embodiments hereof. 1. The account header area shows the following info: user's name, photo/avatar, role, hospital name, and logo. 2. A settings link to update profile and account settings is located in the top right corner of the header area. 3. A dropdown menu allows the user to change their status (Available, Away, Do not disturb), and unit (unit name will depend on hospital). 4. A user dashboard area provides a performance chart (e.g., weekly performance) and other data points. 5. Other links (Send feedback, tutorials, Touch ID & PIN) will take the user to more detailed views on those areas. 6. A link to sign out of the app will be listed at the bottom of the page.

Doctor/Anesthesiologist/OR Nurse Flow

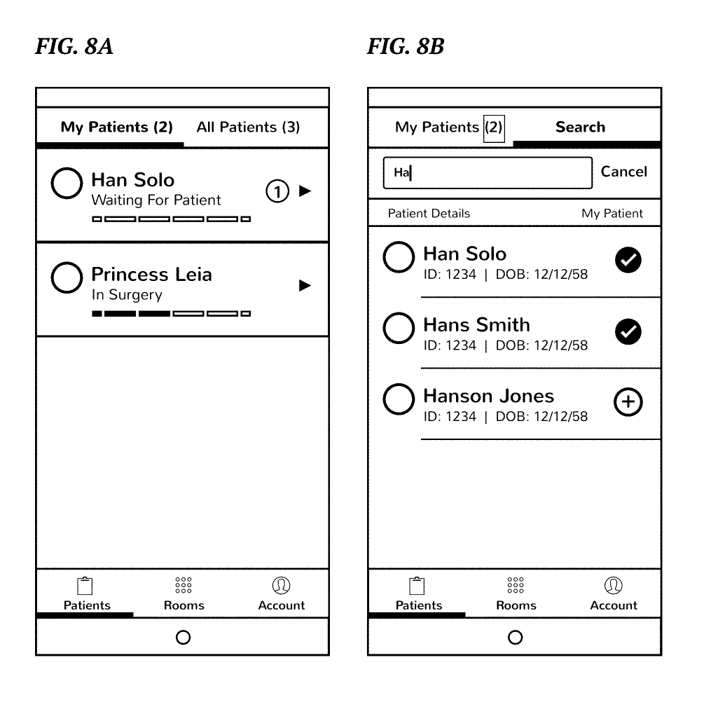

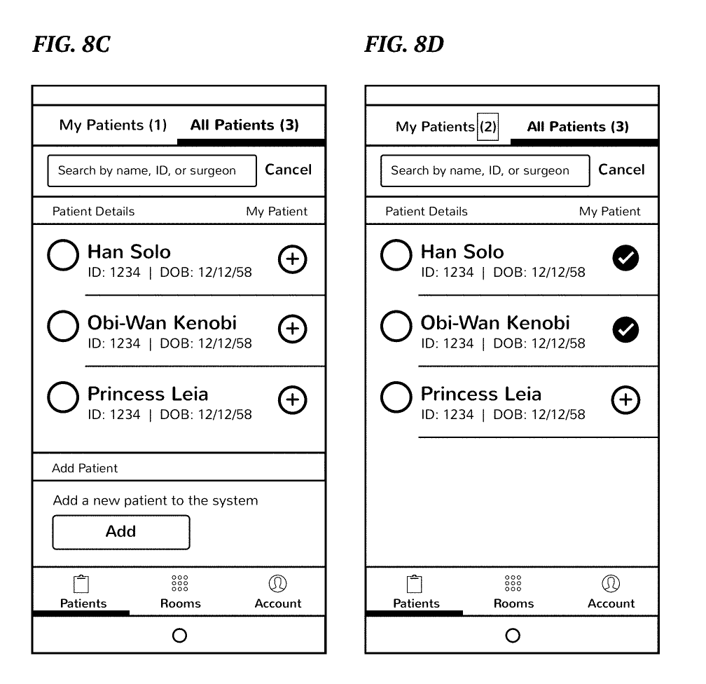

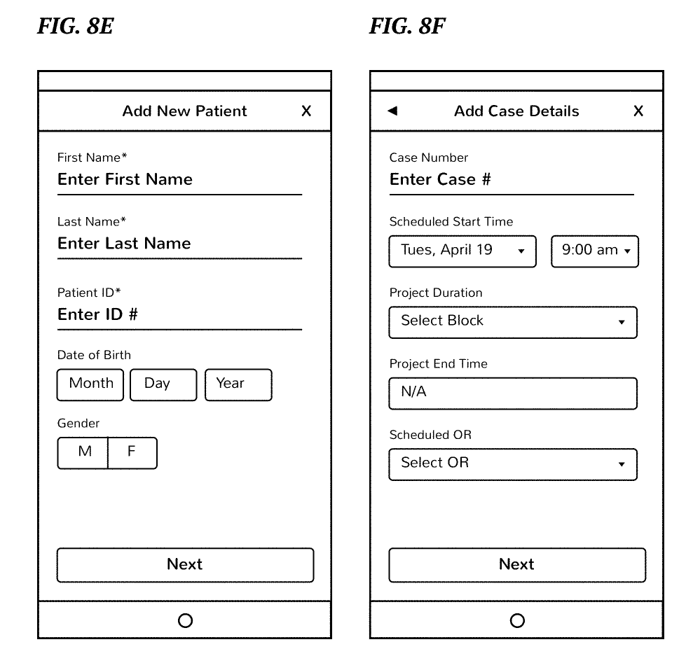

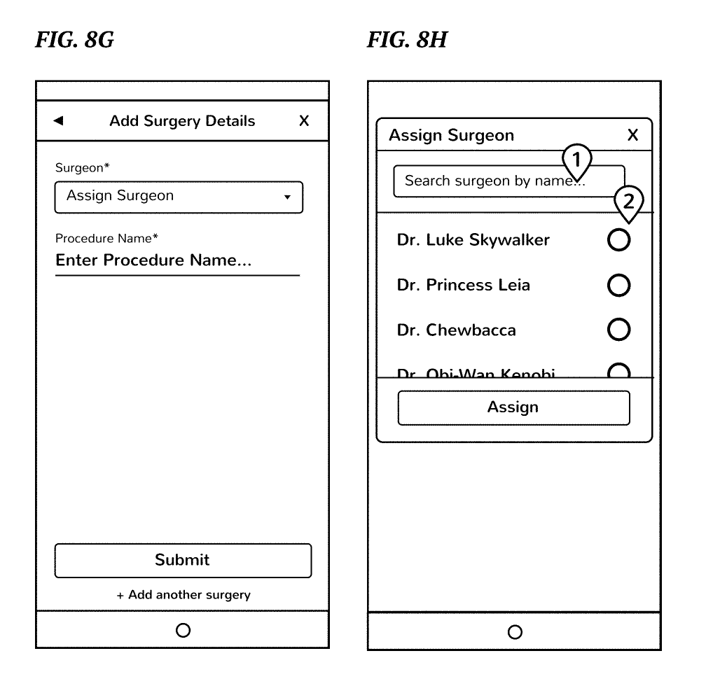

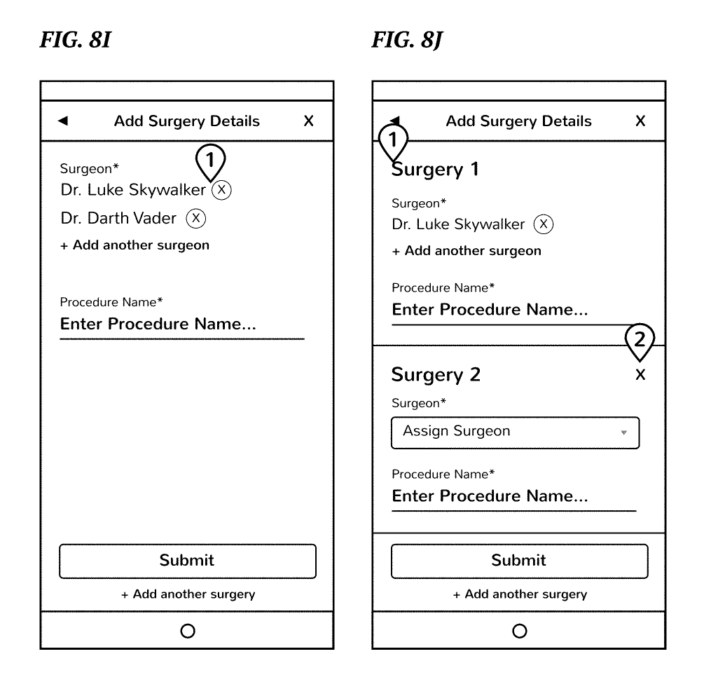

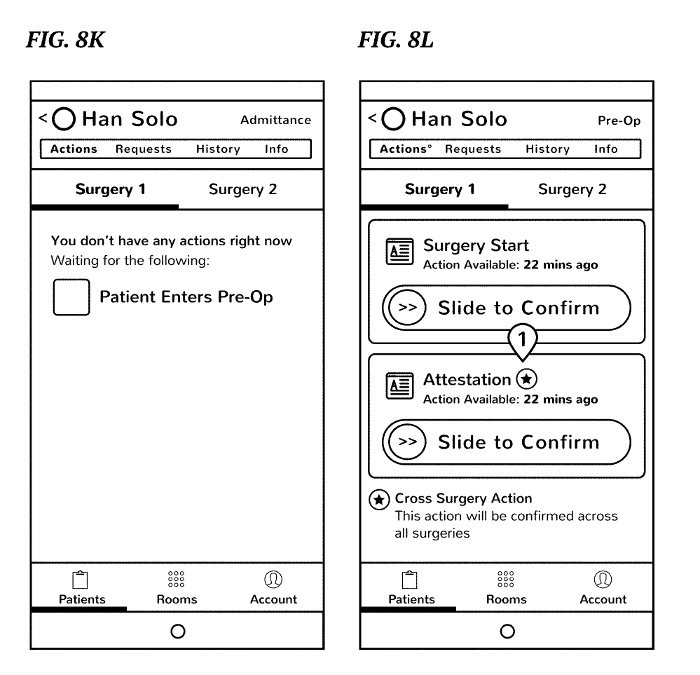

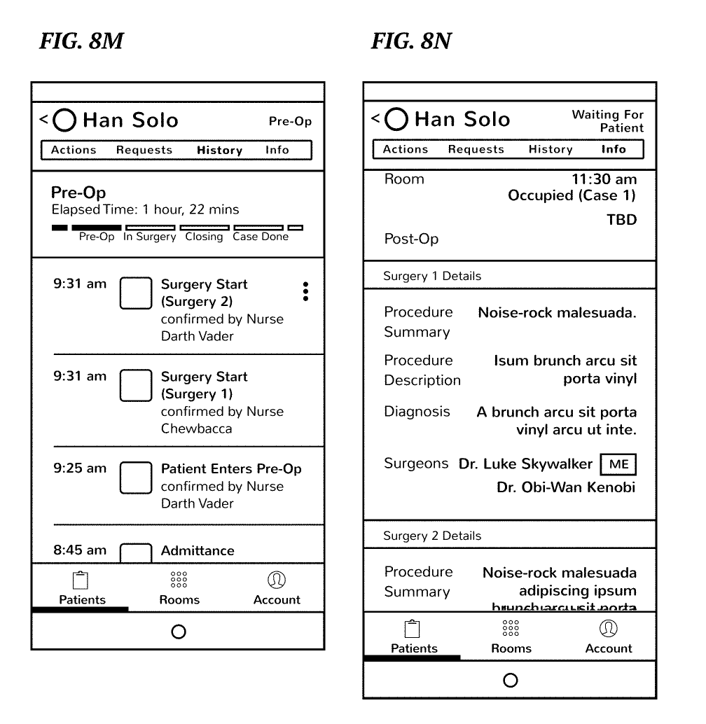

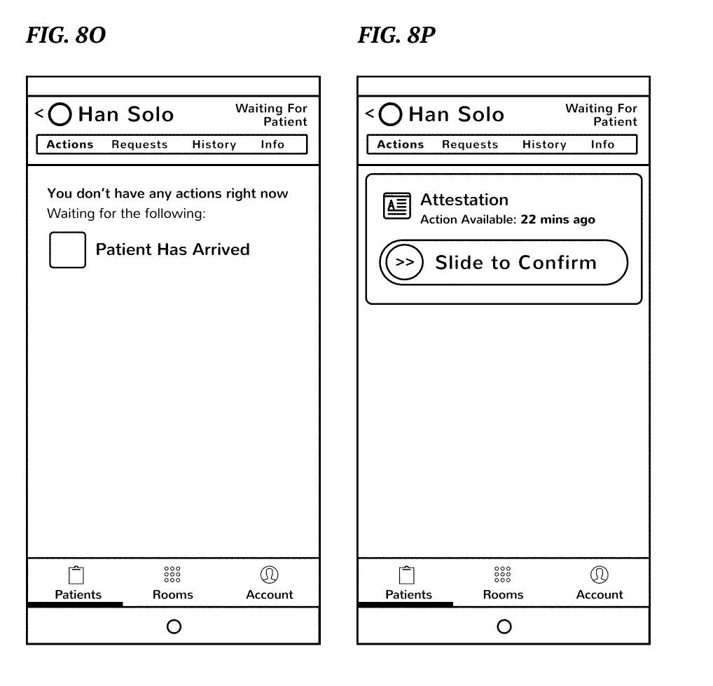

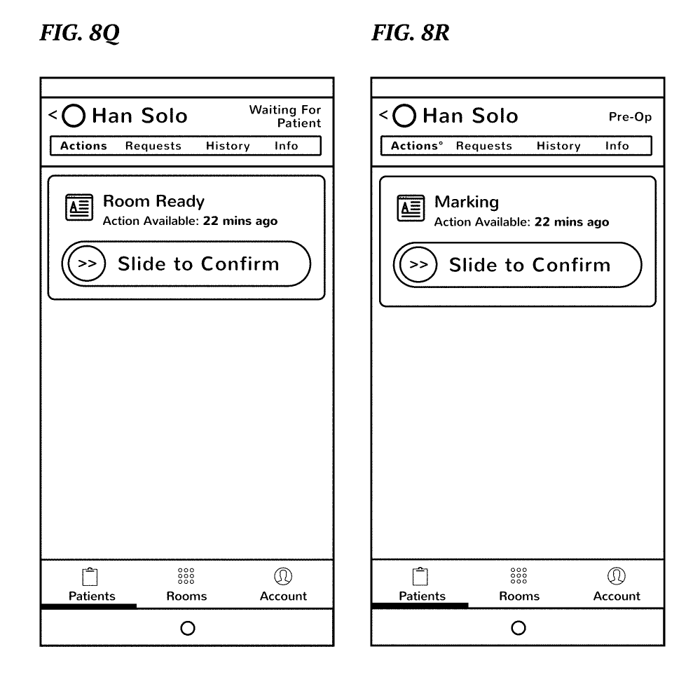

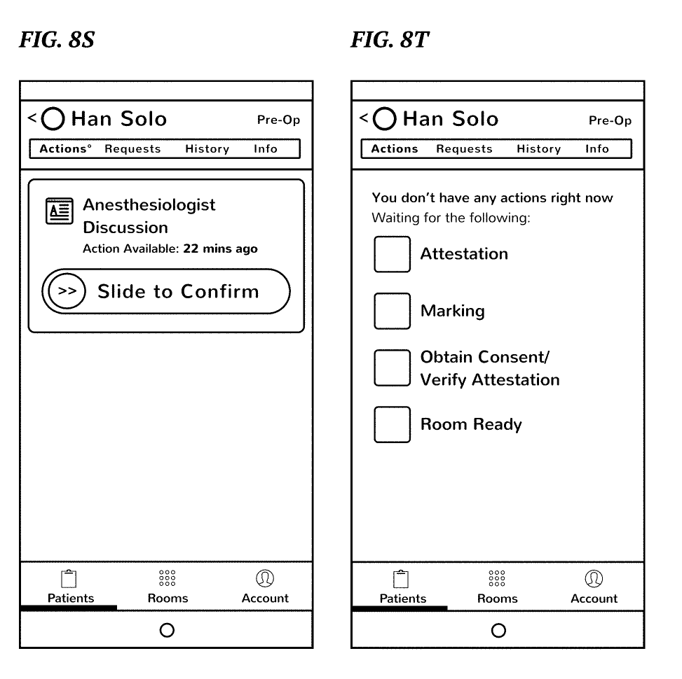

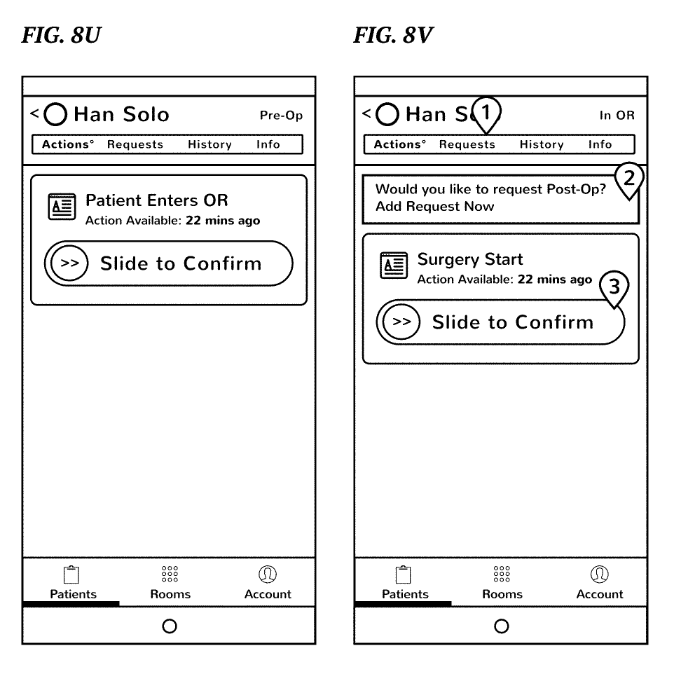

FIGS. 8A-8V, 9A-9X, and 10A-10P depict aspects of a doctor/anesthesiologist/OR nurse flow according to exemplary embodiments hereof.

Doctor/Anesthesiologist/OR Nurse Flow—My Patients

FIG. 8A depict aspects of a My Patient tab (in a doctor/anesthesiologist/OR nurse flow view) according to exemplary embodiments hereof.

In the tab depicted in FIG. 8A, patients added to the My Patient list will show the following patient info: Patient name, current status, and gender icon. If an action is available for the patient, a number badge will appear in the row. Selecting anywhere in the row will take the user to the patient detail page where actions may be performed. Note that if there are no assigned patients, the tab may state “No patients. We will notify you when you have patients in your practice.” or a similar message.

Doctor Flow—Search all Patients

FIG. 8B depicts aspects of a search all patients tab (in a doctor/surgeon flow view) according to exemplary embodiments hereof. FIG. 8B The search box allows the user to search all patients by: patient name, ID, or doctor/surgeon, appearing in the list area below. An empty state message will appear in the search results display area until the user types into the search field. If the user (may only apply to residents) has administrative rights to admit patients, the add new patient option will appear. The search results display shows details of each patient. The user may add a patient to the My Patients view akin to “following” a patient and receiving updates. Selecting the My Patient checkbox in the list will add/remove the patient to My Patient list. In addition, the number counter in the My Patient tab bar will highlight to show if a patient has been added or removed from the list.

Anesthesiologist/OR Nurse Flow—all Patients

FIGS. 8C-8D depicts aspects of an all patients tab (in an anesthesiologist/OR nurse flow view) according to exemplary embodiments hereof.

FIG. 8C The search box allows the user to search all patients by: patient name, ID, or doctor/surgeon, with dynamic filtering of the list below. The display shows details of each patient. An OR Nurse may add a patient to the My Patients view akin to “following” a patient and receiving updates. If the user has administrative rights to admit patients, the add new patient option will appear.

FIG. 8D selecting the My Patient checkbox in the list will add/remove the patient to My Patient list. In addition, the number counter in the My Patient tab bar will highlight to show if a patient has been added or removed from the list.

Doctor/Anesthesiologist/OR Nurse Flow—Add New Patient

FIGS. 8E-8J depicts aspects of an “Add New Patient” flow (in an doctor/anesthesiologist/OR nurse flow view) according to exemplary embodiments hereof. If the user has administrative rights to admit patients, the user is able to enter patient details, case details, and surgery details.

FIG. 8E shows entry fields for adding the patient's details

FIG. 8F shows entry fields for adding the patient's case details

FIG. 8G shows entry fields for adding the patient's surgery details

FIG. 8H: The user will be able to assign a surgeon(s) to the new patient with the assign surgeon modal. 1. Search surgeons by name. List dynamically filters as the user types. 2. Selecting the surgeon name row will select a surgeon to be assigned. Multiple surgeons may be selected in the list.

FIG. 8I: 1. Assigned surgeons will appear in the entry field once selected in the assign surgeon modal. If multiple surgeons are selected, the names will stack in a list. Surgeons may be deleted individually by selecting the delete icon next to the surgeon's name.

FIG. 8J: 1. Multiple surgeries will be separated by section. The surgery number will be listed at the top of each section. 2. Selecting the delete icon will delete the section.

Doctor/Anesthesiologist/OR Nurse Flow—Multiple Procedures

FIGS. 8K-8N show aspects of “Multiple Procedures” in the Patient Details view (in a doctor/anesthesiologist/OR nurse flow view) according to exemplary embodiments hereof.

FIG. 8K shows the patient detail action page (with no actions available) relating to multiple procedures. The user may toggle between concurrent procedures by selecting the applicable tab. If an action is needed on a procedure not in tab view, a notification alert dot will appear in the tab. Note: The doctor/surgeon only see the tabs if they are assigned to the procedure. The nurse(s) are able to see all procedure tabs.

FIG. 8L shows the patient detail action page (with actions) relating to multiple procedures. 1. If a cross-surgery action is available, the action card will be annotated with the cross-surgery icon. Confirming this action will be confirmed across all surgeries.

FIG. 8M shows the patient detail history page relating to multiple procedures. Recorded events for multiple procedures will be listed with the surgery number next to the recorded action title.

FIG. 8N: Multiple procedures are preferably listed in separate sections on the patient details info page. Corresponding details are displayed in each section. If multiple surgeons are assigned to a procedure, all surgeon names will be listed in the corresponding procedure section.

Doctor/Anesthesiologist/OR Nurse Flow—Patient Details

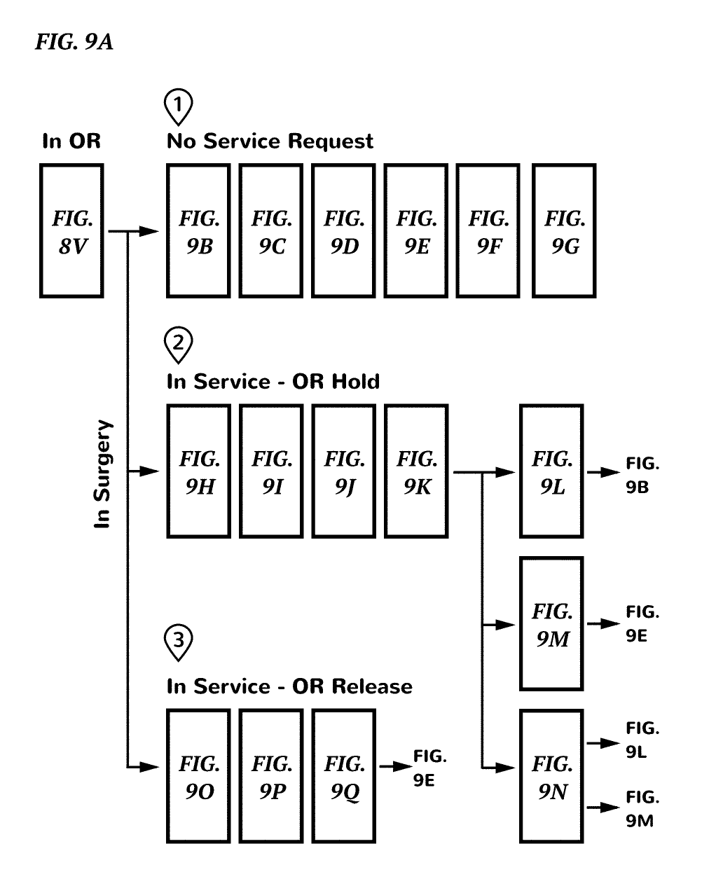

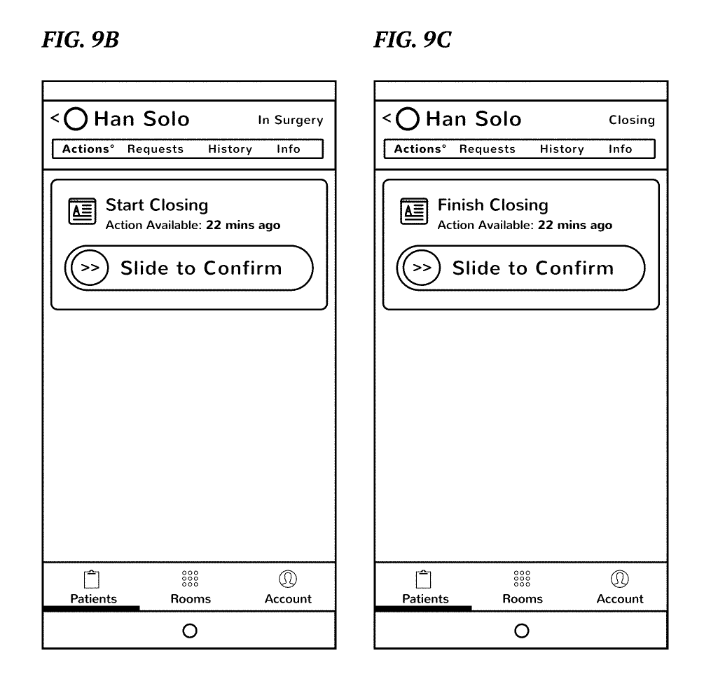

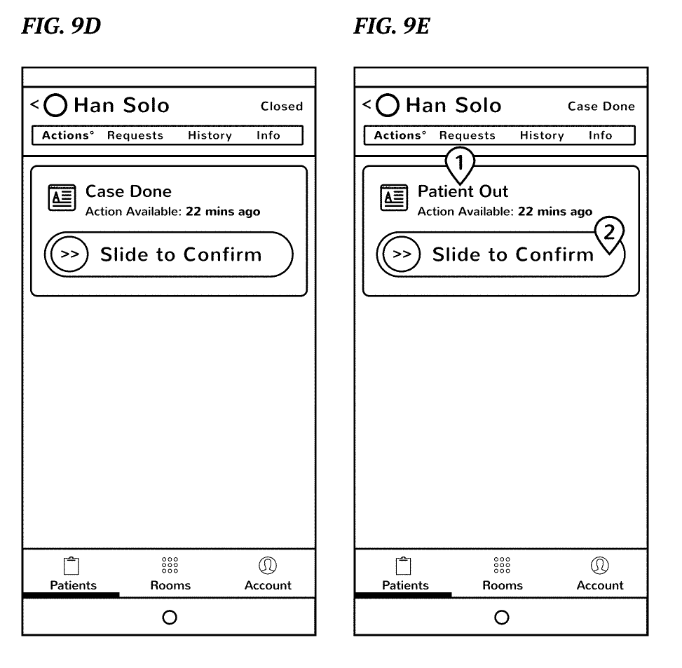

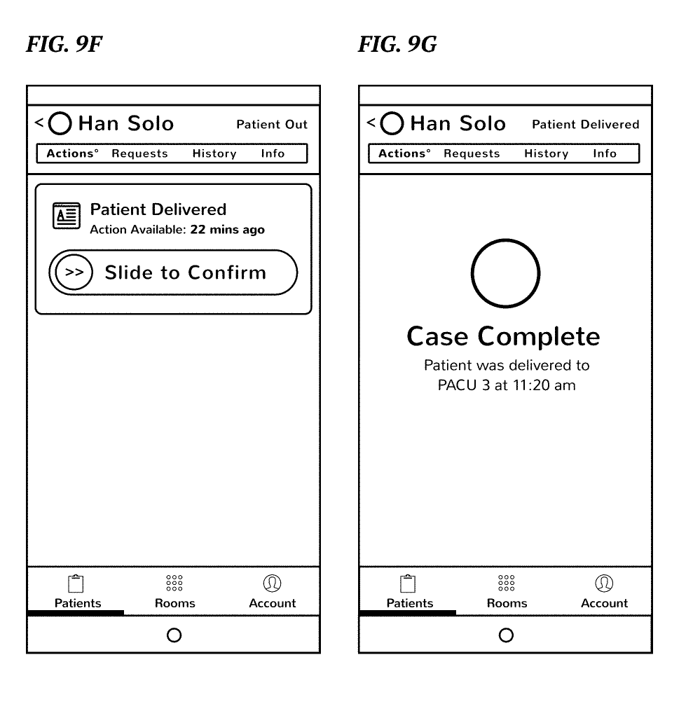

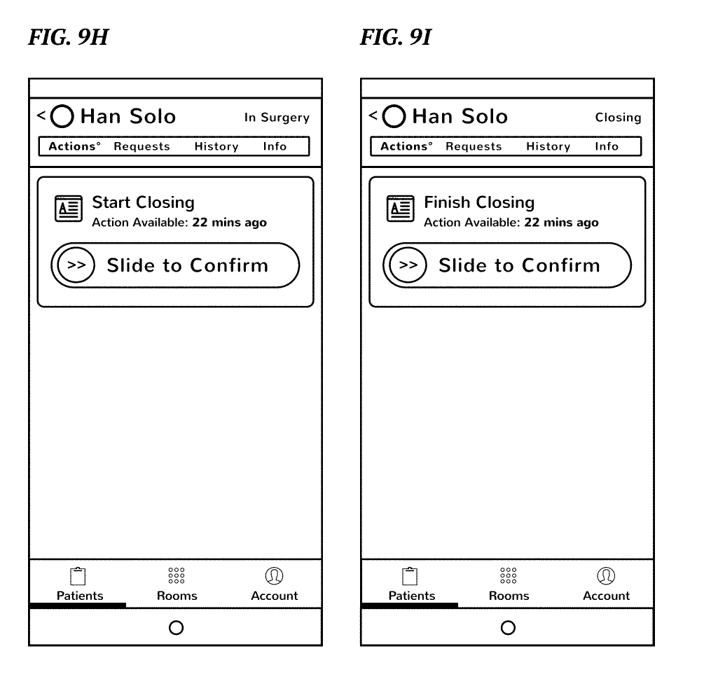

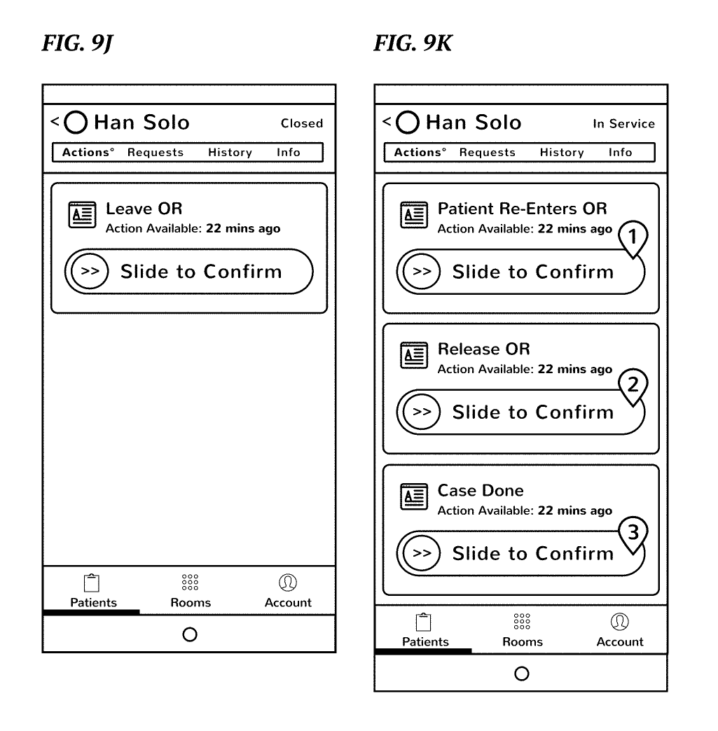

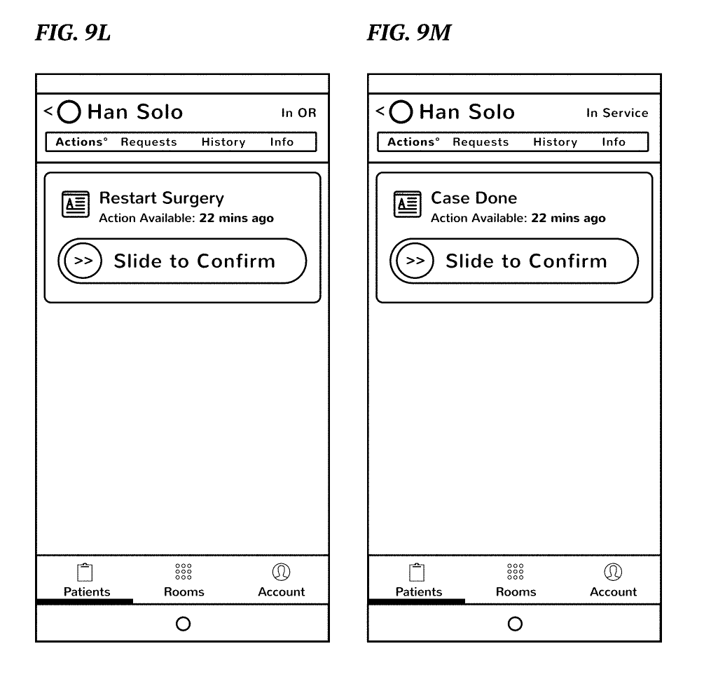

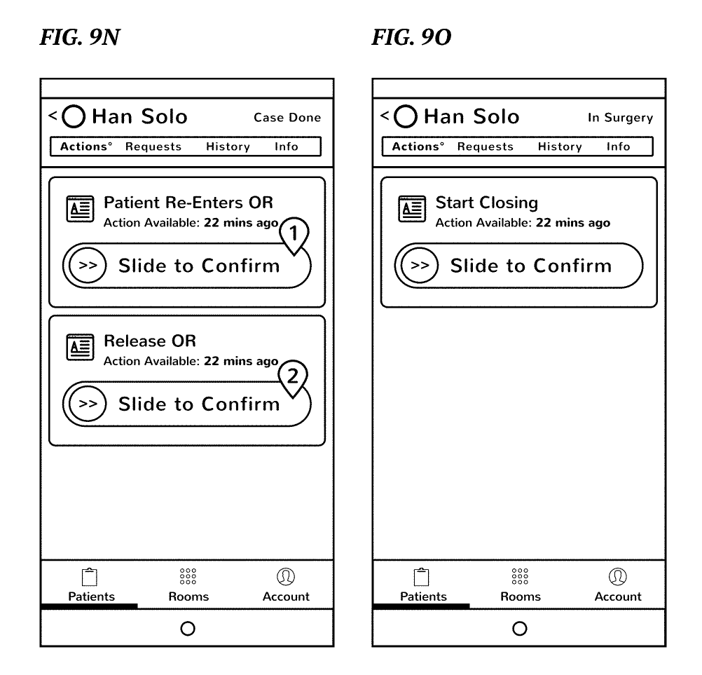

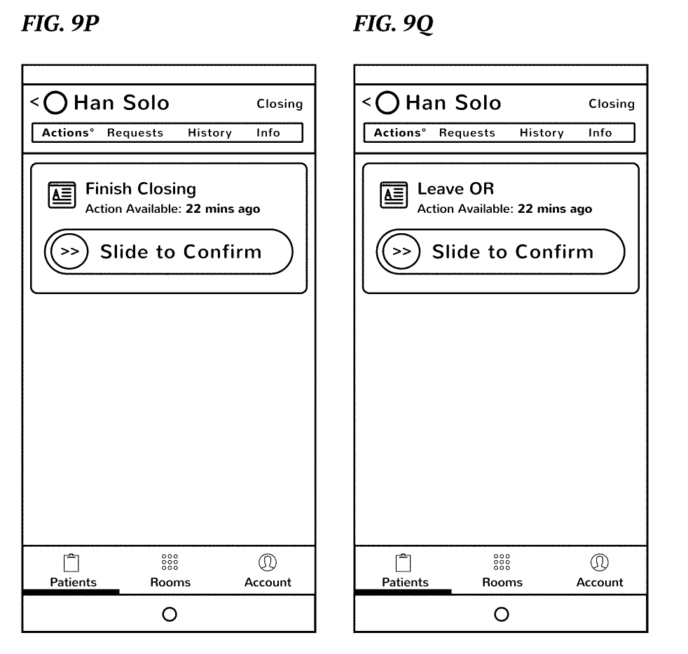

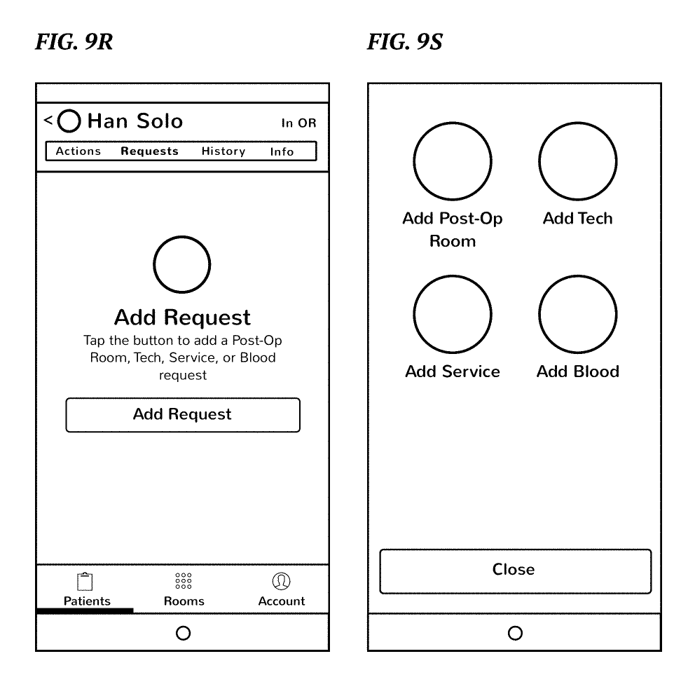

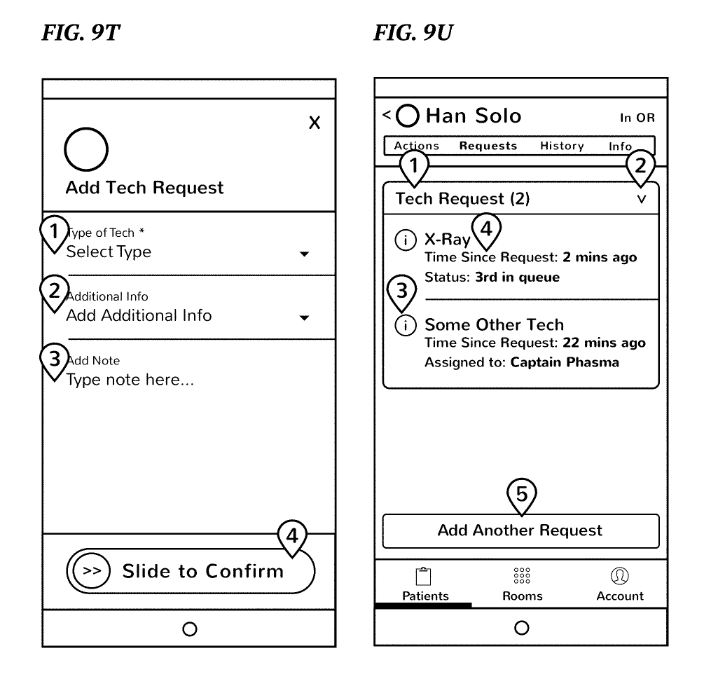

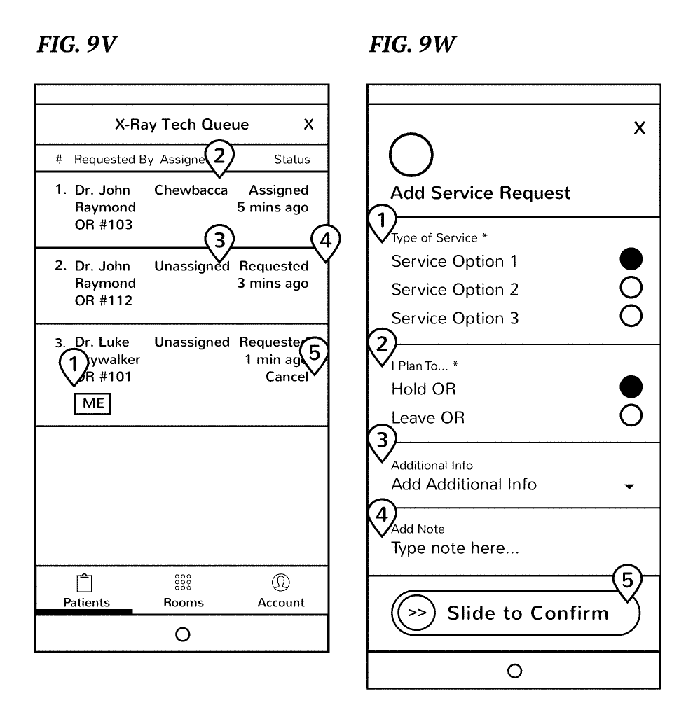

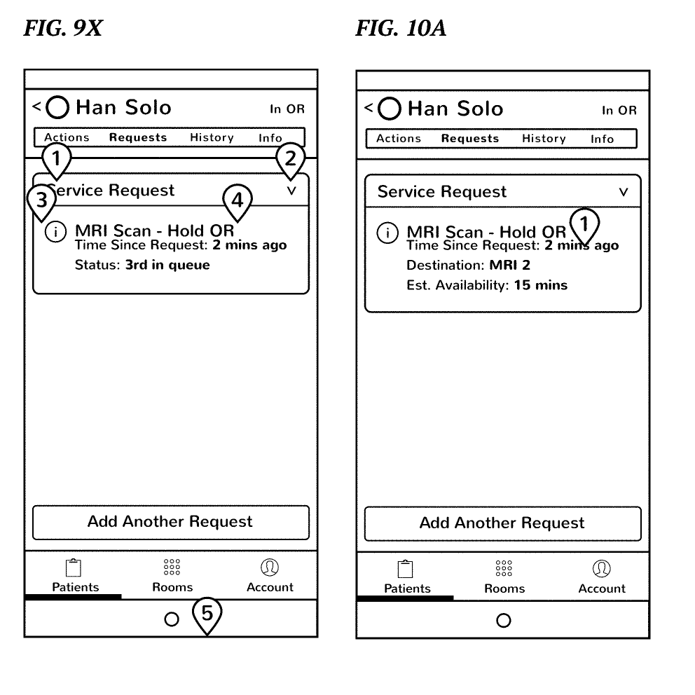

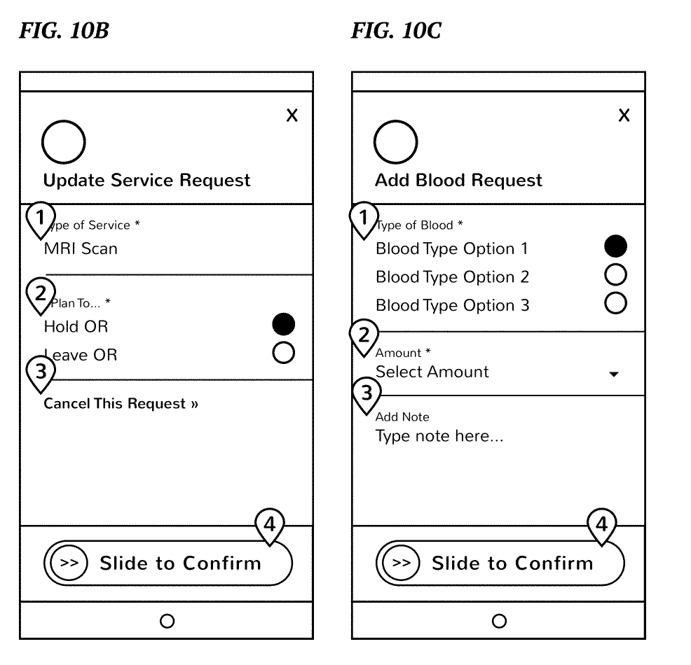

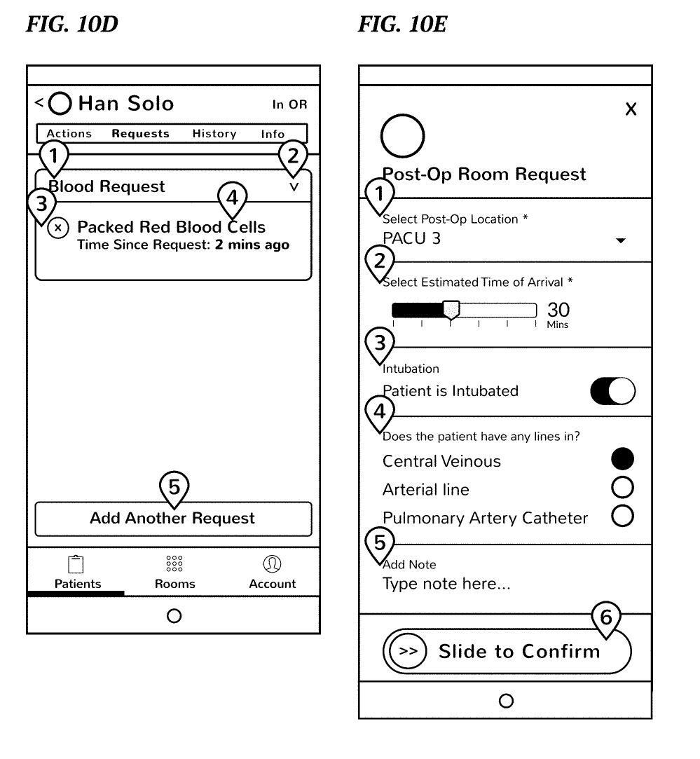

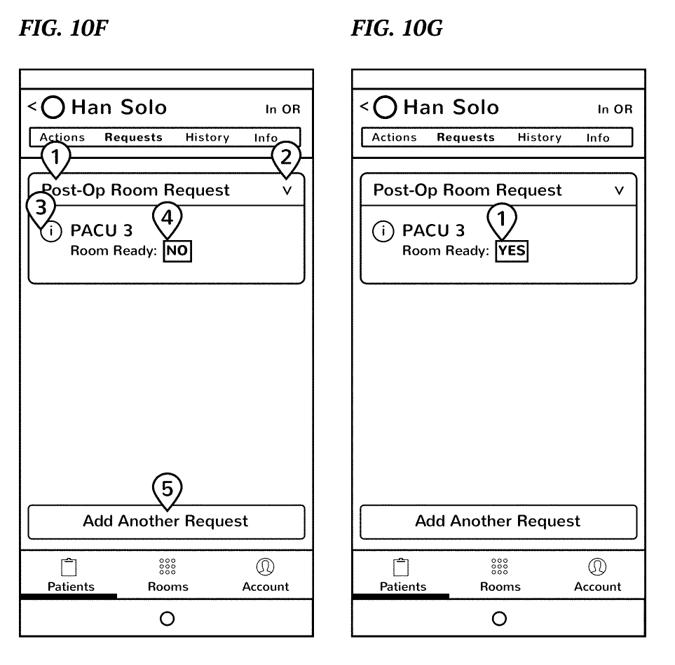

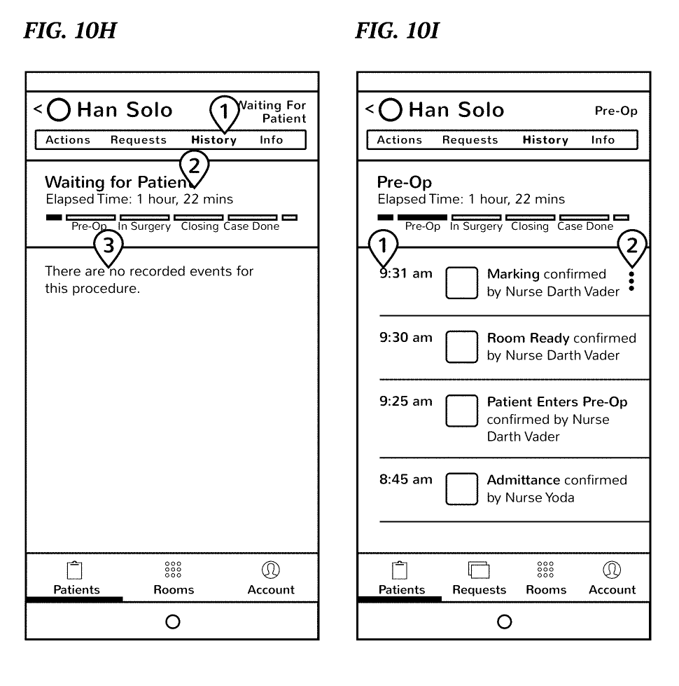

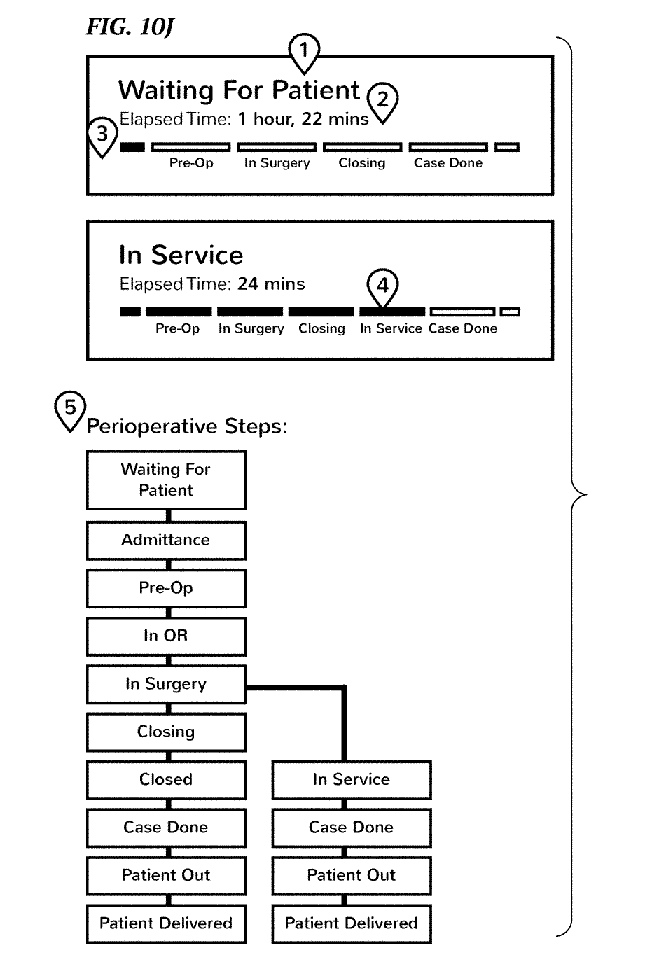

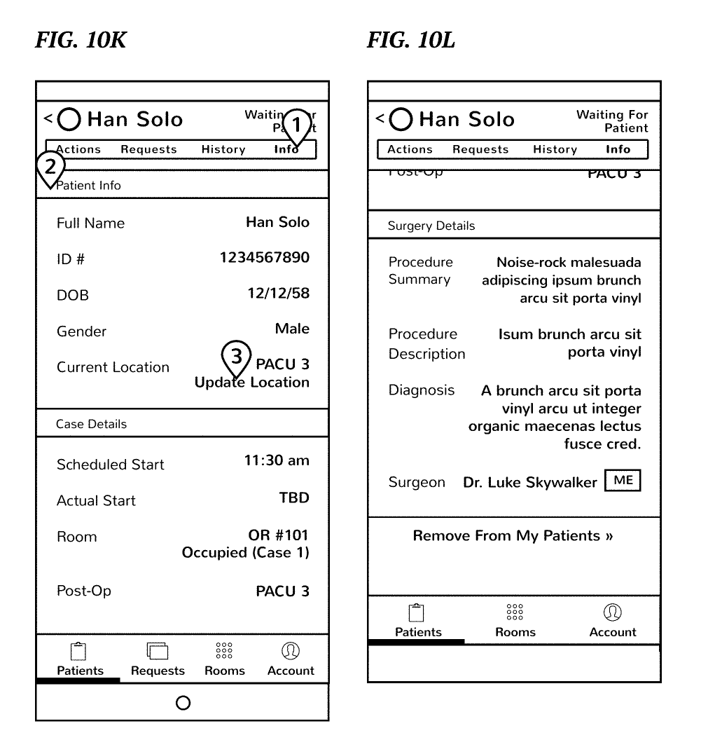

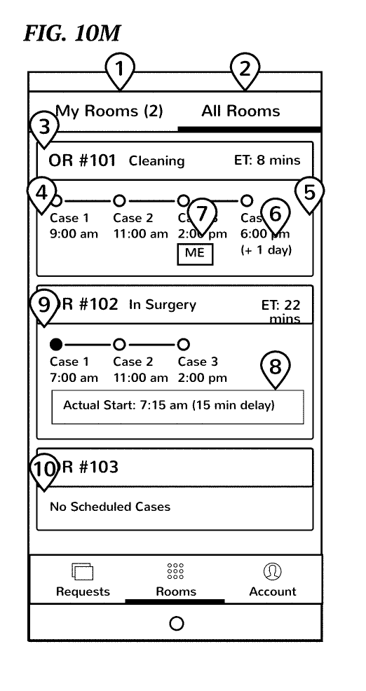

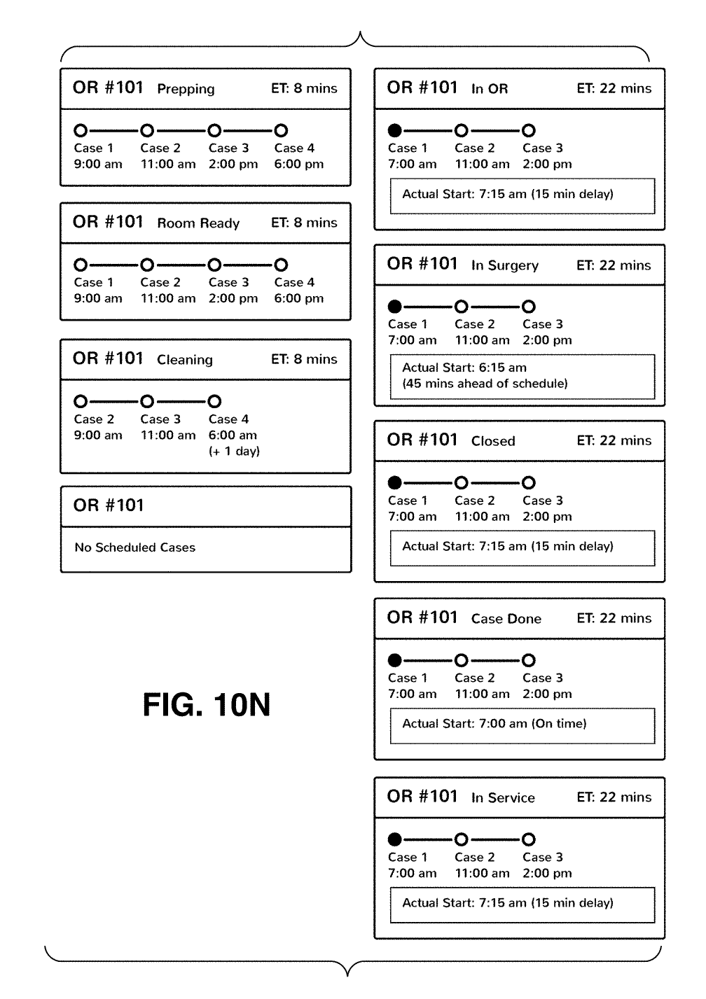

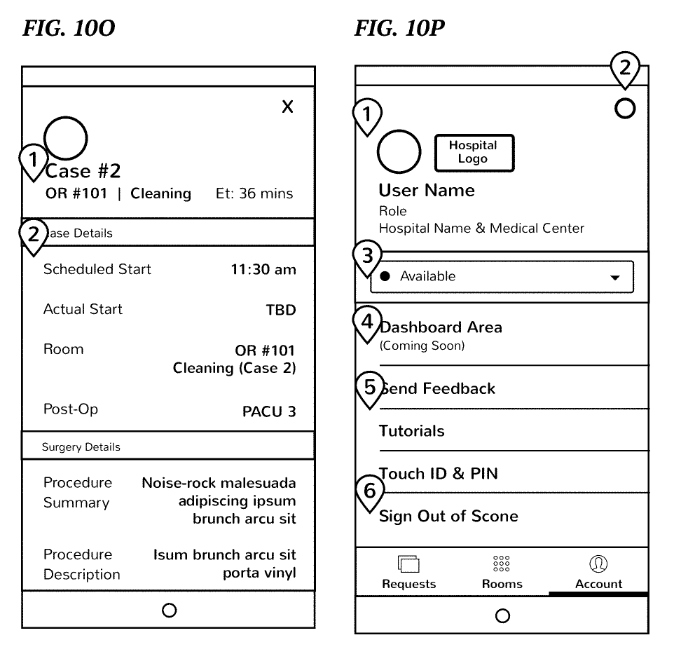

FIGS. 8O-8V, 9A-9X, and 10A-10P depict aspects of a “Patient Detail” (in a doctor/anesthesiologist/OR nurse flow view) according to exemplary embodiments hereof. The top section of the page displays the following patient details: Patient name, gender icon, patient status, and segment controller with multiple options. The user may select in the segment controller to toggle between: actions (default), requests, history, and info. If an action is available, show an alert dot next to the actions text. The user may also return to the previous patient list view by selecting the back arrow at the top of the screen. In the main actions view, an active Action card shows: Action title/icon, and how long the action has been available. The interface uses a slide button interaction to confirm task. The card will slide away once confirmed. The following table summarizes aspects of the tabs and user interface:

| FIG. Number | Description |
|---|---|
| 8O | Waiting for patient |
| | If there is no immediate action by the user, show a waiting message and the |
| | action(s) that the user is waiting to be completed. Once completed, the next |
| | action will appear |
| 8P | Attestation (doctor/surgeon only) |
| | Confirm attestation is available for the doctor/surgeon to take action. Slide |
| | button interaction to confirm task. Slide button interaction to confirm task. |
| | The card will slide away once confirmed. (Note: attestation may be available |
| | to confirm before the patient has arrived) |
| | If there is no immediate action by the anesthesiologist/OR nurse, show a |
| | waiting message and the action(s) that the user is waiting to be completed. |
| | Once completed, the next action will appear. (Note: attestation may be |
| | available to confirm before the patient has arrived) |
| 8Q | Room Ready (OR nurse only) |
| | Confirm room ready is available for the OR nurse to take action. Slide button |
| | interaction to confirm task. Slide button interaction to confirm task. The card |
| | will slide away once confirmed. (Note: the room ready action may be |
| | available to confirm before the patient has arrived) |
| | If there is no immediate action by the doctor/anesthesiologist, show a waiting |
| | message and the action(s) that the user is waiting to be completed. Once |
| | completed, the next action will appear. (Note: room ready may be available to |
| | confirm before the patient has arrived) |
| 8R | Marking (doctor/surgeon only) |
| | Confirm marking is available for the doctor/surgeon to take action. Slide |
| | button interaction to confirm task. Slide button interaction to confirm task. |
| | The card will slide away once confirmed. |
| | If there is no immediate action by the anesthesiologist/OR Nurse, show a |
| | waiting message and the action(s) that the user is waiting to be completed. |
| | Once completed, the next action will appear |
| 8S | Anesthesiologist Discussion (Anesthesiologist only) |
| | Confirm anesthesiologist discussion is available for the anesthesiologist to |
| | take action. Slide button interaction to confirm task. The card will slide away |
| | once confirmed. |
| | If there is no immediate action by the doctor/OR Nurse, show a waiting |
| | message and the action(s) that the user is waiting to be completed. Once |
| | completed, the next action will appear |
| 8T | Pre-Op: waiting for actions |
| | If there is no immediate action by the user, show a waiting message and the |
| | action(s) that the user is waiting to be completed. Once completed, the next |
| | action will appear |
| 8U | Patient enters OR |
| | Confirm patient enters OR is available for the user to take action. Slide button |
| | interaction to confirm task. Slide button interaction to confirm task. The card |
| | will slide away once confirmed. |
| 8V | Surgery start |
| | 1. The user may add requests (Tech, Service, Blood, Post-Op Room) once the |
| | patient is in the OR by selecting the requests tab. |
| | 2. The user may request a Post-Op room before closing has started (if needed) |
| | by selecting the message banner, or by selecting the requests tab. Selecting a |
| | Post-Op destination will trigger a notification alert to the appropriate teams |
| | (ex: cleaning crew/transport/post-op nurse) that a patient will be finishing |
| | soon. Further, if there is a pending service request w/an OR hold, the |
| | notification will be suppressed. |
| | 3. Confirm surgery start is available for the user to take action. Slide button |
| | interaction to confirm task. The card will slide away once confirmed. |
| 9A | In surgery overview |
| | The in surgery overview displays several possible flows while the patient is in |
| | the OR. |
| | 1. If there are no service requests, proceed to In Surgery (FIG. 9B.) |
| | 2. If there is a service request with OR Hold, proceed to In Surgery - OR Hold |
| | (FIG. 9H) |
| | 3. If there is a service request with OR Release, proceed to In Surgery - OR |
| | Release (FIG. 9O) |
| 9B | In Surgery |
| | Confirm start closing is available for the user to take action. Slide button |
| | interaction to confirm task. The card will slide away once confirmed. |
| 9C | Closing in progress |
| | Confirm finish closing is available for the user to take action. Slide button |
| | interaction to confirm task. The card will slide away once confirmed. |
| 9D | Closed |
| | Confirm case done is available for the user to take action. Slide button |
| | interaction to confirm. The card will slide away once confirmed. |
| 9E | Case Done |
| | 1. Confirm patient out is available for the user to take action. Slide button |
| | interaction to confirm task. The card will slide away once confirmed. |
| | 2. After patient out is confirmed, if there are still open requests a prompt will |
| | appear reminding the user to either leave all requests open, or close all. If |
| | there are no open requests, proceed to patient out (FIG. 9F) |
| | Note that if there are still open requests in-progress, a dialog box may appear |
| | confirming this and giving the user an option to either leave the requests open |
| | or to close them. |
| 9F | Patient Out |
| | Confirm patient delivered is available for the user to take action. Slide button |
| | interaction to confirm task. The card will slide away once confirmed. |
| 9G | Patient Delivered |
| | Once the patient is delivered, the following info will be displayed in the |
| | actions area: Case complete message w/icon, post-op unit delivered, and |
| | timestamp delivered |
| 9H-9N | Service Request - OR Hold |
| | FIGS. 9H-9N depict aspects of a service requested during surgery with an OR |
| | Hold, according to exemplary embodiments hereof. |
| 9H | In Surgery- OR Hold |
| | Confirm start closing is available for the user to take action. Slide button |
| | interaction to confirm task. The card will slide away once confirmed. Once |
| | confirmed, the patient will proceed to Closing (FIG. 9I) |
| 9I | Closing - OR Hold |
| | Confirm finish closing is available for the user to take action. Slide button |
| | interaction to confirm task. The card will slide away once confirmed. Once |
| | confirmed, the patient will proceed to Closed (FIG. 9J) |
| 9J | Closed - OR Hold |
| | Confirm leave OR is available for the user to take action. Slide button |
| | interaction to confirm task. The card will slide away once confirmed. Once |
| | confirmed, the patient will proceed to In Service (FIG. 9K) |
| 9K | In Service - OR Hold |
| | Update the patent status to “In Service.” |
| | The following actions are available for the user to confirm: |
| | 1. Patient Re-Enters OR - If confirmed, proceed to Re-start surgery (FIG. 9L) |
| | 2. Release OR - If confirmed, proceed to the open request prompt if there are |
| | open requests, otherwise proceed to OR Release Case Done (FIG. 9N) |
| | 3. Case Done - If confirmed, proceed to Confirm OR (FIG. 9N) |
| 9L | Patient Re-Enters OR - OR Hold (From FIG. 9K) |
| | Confirm Restart surgery is available for the user to take action. Slide button |
| | interaction to confirm task. The card will slide away once confirmed. Once |
| | confirmed, the patient will proceed to In surgery (FIG. 9B) |
| 9M | Release OR (From FIG. 9K) |
| | Confirm Case Done is available for the user to take action. Slide button |
| | interaction to confirm task. The card will slide away once confirmed. Once |
| | confirmed, the patient will proceed to confirm patient out (FIG. 9E) |
| 9N | Case Done - OR Hold (From FIG. 9K) |
| | The following actions are available for the user to confirm: |
| | 1. Patient Re-Enters OR - If confirmed, proceed to Re-start surgery (FIG. 9L) |
| | 2. Release OR - If confirmed, proceed to the open request prompt if there are |
| | open requests, otherwise proceed to OR Release Case Done (FIG. 9M) |
| 9O-9Q | Service request - OR Release |
| | FIGS. 9O-9Q depict aspects of a service requested during surgery with an OR |
| | Release, according to exemplary embodiments hereof. |
| 9O | In Surgery- OR Release |
| | Confirm start closing is available for the user to take action. Slide button |
| | interaction to confirm task. The card will slide away once confirmed. Once |
| | confirmed, the patient will proceed to Closing (FIG. 9P) |
| 9P | Closing - OR Release |
| | Confirm finish closing is available for the user to take action. Slide button |
| | interaction to confirm task. The card will slide away once confirmed. Once |
| | confirmed, the patient will proceed to Closed (FIG. 9Q) |
| 9Q | Closed - OR Release |
| | Confirm leave OR is available for the user to take action. Slide button |
| | interaction to confirm task. The card will slide away once confirmed. Once |
| | confirmed, the patient will proceed to confirm patient out (FIG. 9E) |
| 9R-10G | Requests |
| | FIGS. 9R-10G depict aspects of requests in the “Patient Detail” (in an |
| | doctor/anesthesiologist/OR nurse flow view) according to exemplary |
| | embodiments hereof. |
| 9R | Requests available |
| | Requests are available once the patient enters the OR. Prior to this a dialog |
| | may state that requests are not yet available. If the patient is in the OR, an |
| | initial request has not been made, show a requests available message with |
| | CTA button to add a request. The user may continue to add requests as needed |
| | throughout the OR. |
| 9S | Request chooser overlay |
| | Available request types will appear in the chooser. Select the circle icon/text |
| | to proceed to the applicable request form. Some requests may be disabled if |
| | concurrent requests of the same type are not allowed (Note: Only 1 Tech |
| | request type, and 1 service request is allowed per patient. Multiple blood |
| | requests are allowed per patient) |
| | The user may select the close button to exit the chooser. |
| 9T | Tech request form |
| | 1. The user selects an available tech (List will be defined per hospital) from |
| | the list. (required); Note: Required fields will be noted with an asterisk |
| | 2. Additional info may be entered (optional) by the user. |
| | 3. The user may add a note (optional). |
| | 4. Slide button interaction to create request. Slider will be disabled until all |
| | required fields are filled |
| 9U | Tech request card created |
| | 1. Once a Tech request is made, the request type title is displayed in the task |
| | card. |
| | 2. Request cards may be collapsed to hide details (expanded by default) |
| | 3. Selecting the info icon, or anywhere in the row surfaces the specified tech |
| | queue |
| | 4. The request shows the following info: Type of request, waiting time, status |
| | 5. Selecting the add another request button opens the request chooser overlay |
| | (FIG. 9S) where the user may create another request. |
| 9V | Tech request queue (X-Ray) |
| | 1. My requests will include the “Me” badge in the requested by info |
| | 2. An accepted request will show the name of the tech who accepted the |
| | request |
| | 3. Unassigned requests will show “unassigned” |
| | 4. An unassigned request will be highlighted in the list in a different color |
| | 5. A link to cancel the request is shown only for my requests. The user may |
| | cancel the request after a confirmation dialog appears. |
| 9W | Service request form |
| | 1. The user selects an available service (List will be defined per hospital) from |
| | the list (required) |
| | 2. The user selects a post-service option: Leave and Hold OR/Leave and |
| | Release OR. (required) |
| | 3. Additional info may be entered (optional) by the user |
| | 4. The user may add a note (optional). |
| | 5. Slide button interaction to create request. Slider will be disabled until all |
| | required fields are filled |
| 9X | Service request card created (request not responded) |
| | 1. Once a Service request is made, the request type title is displayed in the task |
| | card. |
| | 2. Request cards may be collapsed to hide details (expanded by default) |
| | 3. Selecting the info icon, or anywhere in the row surfaces the update service |
| | request form |
| | 4. The request shows the following info: Type of request, waiting time, status. |
| | If a OR hold is requested, update the room status in the patient info area. |
| | 5. Selecting the add another request button opens the request chooser overlay |
| | (FIG. 9S) where the user may create another request. |
| 10A | Service request card created (request responded) |
| | 1. Service details (destination, estimated availability) will automatically |
| | update in the request card when the service request is responded to by the |
| | service manager |
| 10B | Update service request form |
| | 1. Once a service request is created, the type of service is not editable. |
| | 2. Ability to update Hold/Release OR is editable (required). |
| | 3. Selecting the cancel link will cancel the service request. The user may |
| | cancel the request after a confirmation dialog appears. |
| | 4. Slider is disabled until a change is detected |
| 10C | Blood request form |
| | 1. The user selects the type of blood needed: Packed Red Blood Cells, Fresh |
| | Frozen Plasma, Whole Blood, Fancy stuff, etc., depending on hospital |
| | inventory (required); |
| | 2. The user selects the amount of blood needed depending on blood type: |
| | Units, Packs, Vials, etc. (required) |
| | 3. The user may add a note (optional). |
| | 4. Slide button interaction to create request. Slider will be disabled until all |
| | required fields are filled |
| 10D | Blood request card created |
| | 1. Once a Blood request is made, the request type title is displayed in the task |
| | card. |
| | 2. Request cards may be collapsed to hide details (expanded by default) |
| | 3. Selecting the cancel icon will cancel the blood request. The user may cancel |
| | the blood request after a confirmation dialog appears. |
| | 4. The request shows the following info: Type of request, waiting time |
| | 5. Selecting the add another request button opens the request chooser overlay |
| | (FIG. 9S) where the user may create another request. |
| 10E | Post-Op room request form |
| | 1. The user selects an available post-op location (List will be defined per |
| | hospital) from the list (Required) |
| | 2. ETA: Drag slider to update ETA (required) |
| | 3. Intubation: Select toggle to yes/no (optional) |
| | 4. Select checkboxes to provide additional info (provided by hospital) |
| | (optional) |
| | 5. Select field to add a note (optional) |
| | 6. Slide button interaction to create request. Slider will be disabled until all |
| | required fields are filled |
| 10F | Post-Op room request card created (room not ready) |
| | 1. Once a post-op room request is made, the request type title is displayed in |
| | the task card. |
| | 2. Request cards may be collapsed to hide details (expanded by default) |
| | 3. Selecting the info icon, or anywhere in the row surfaces the update request |
| | form. The user may update the Post-Op destination up until closing has |
| | finished. |
| | 4. The request shows the following info: Type of request, room status = |
| | No/Yes |
| | 5. Select the button to add another request |
| 10G | Post-Op room request card created (room ready) |
| | 1. Show current room status with background highlight box: No = red box, |
| | Yes = green box |
| 10H-10J | Full History |
| | depict aspects of full history in the “Patient Detail” (in a |
| | doctor/anesthesiologist/OR nurse flow view) according to exemplary |
| | embodiments hereof. |
| 10H | History - no recorded actions |
| | 1. The full history view is displayed by selecting history in the segment |
| | controller. |
| | 2. The patient status area shows the following info: Current patient |
| | status, elapsed time, and status bar chart with current status highlighted in |
| | green. |
| | 3. If there are no recorded actions, show an empty state message. |
| 10I | History - w/recorded actions |
| | 1. Display completed action: icon, completed/confirmed by, timestamp, and |
| | any other details as needed (examples of most common actions are shown |
| | above but may vary depending on hospital). |
| | 2. Modified actions (undo, skipped) may be accessed by selecting the more icon. |
| 10J | Patient status cards |
| | Patient status area during various stages of the perioperative flow. |
| | 1. The current patient status (current step in the perioperative flow) is listed. |
| | 2. The elapsed time of the current patient status is displayed. |
| | 3. The status bar chart displays the current step in the perioperative status in green. |
| | 4. During a service request, the status card area updates to display the In Service status |
| | title in orange, and the status bar chart expands to include an In Service |
| | section. |
| | 5. The perioperative flow steps may be customized per hospital. |
| 10K-10L | Info |
| | depicts aspects of info in the “Patient Detail” (in a doctor/anesthesiologist/OR |
| | nurse flow view) according to exemplary embodiments hereof. |
| 10K | Patient info display |
| | 1. The info view is displayed by selecting info in the segment controller. |
| | 2. Patient info is grouped by sections: Patient info (Full name, ID, DOB, gender, |
| | current location), Case details (Scheduled start, actual start, room, post-op), |
| | Surgery details (Procedure(s) name, surgeon(s)), Remove from My Patients. |
| | 3. A user may update the current patient location only during Pre-Op and Post- |
| | Op, and not during surgery. If the location is editable, the location link will |
| | appear in blue. |
| 10L | Patient info display (continued) |

Doctor/Anesthesiologist/OR Nurse Flow—My Rooms

FIGS. 10M-10O depicts aspects of a Rooms tab (in a doctor/anesthesiologist/OR nurse flow view) according to exemplary embodiments hereof. Room number will determine card order. In some implementations the room status may be one of “Prepping,” “Cleaning,” “Room Ready,” or occupied states: “In OR,” “In Surgery,” “Closed,” “Case Done,” “In Service.”

With reference to FIG. 10M: In a “My ROOMS: Card View, 1. Room cards with my cases (denoted with a “Me” badge underneath the case info) will appear in the list below. In an “ALL ROOMS: Card View,” 2. Show all room cards in the list below; 3. Room card header shows the following info: OR #, room status, room status elapsed time. The header bar color will be colored according to room status: orange=cleaning, prepping, red=occupied, green=room ready, gray=no scheduled cases; 4. Case # and scheduled time will appear in the display if available. Selecting the case # will display the room detail view (FIG. 10O); 5. The user may swipe to see more cases if needed; 6. If a displayed case is +X days in advance, the text “(in X days)” will appear. 7. My cases will be highlighted with a “Me” badge underneath the case info. 8. In OCCUPIED States (In OR, In Surgery, Closed, Case Done, In Service, OR Hold): the actual start time will be displayed for occupied rooms. The display next to the actual start will show the amount of delay or time ahead of schedule as applicable (delayed/on time/ahead of schedule); 9. For NO SCHEDULED CASES: 1. An empty state message will appear if there are no other cases scheduled for the room.

FIG. 10N depicts aspects of room card status examples in various states: “Prepping,” “Cleaning,” “Room Ready,” or occupied states: “In OR,” “In Surgery,” “Closed,” “Case Done,” “In Service.”

FIG. 10O depicts aspects of a Room Details view (in a doctor/anesthesiologist/OR nurse flow view) according to exemplary embodiments hereof. 1. The room detail header area shows the following info: Case #, room #, room status, room status elapsed time. The header background will be colored according to room status: orange=cleaning, prepping, red=occupied, green=room ready, gray=no scheduled cases; 2. Show the following info (if known): case details, surgery details, patient info (if My Patient).

Doctor/Anesthesiologist/OR Nurse Flow—Account

FIG. 10P depicts aspects of an account tab (in a doctor/anesthesiologist/OR nurse flow view) according to exemplary embodiments hereof. 1. The account header area shows the following info: user's name, photo/avatar, role, hospital name, and logo. 2. A settings link to update profile and account settings is located in the top right corner of the header area. 3. A dropdown menu allows the user to change their status (Available, Away, Do not disturb). 4. A user dashboard area provides a performance chart (e.g., weekly performance) and other data points. 5. Other links (Send feedback, tutorials, Touch ID & PIN) will take the user to more detailed views on those areas. 6. A link to sign out of the app will be listed at the bottom of the page.

Tech Flow

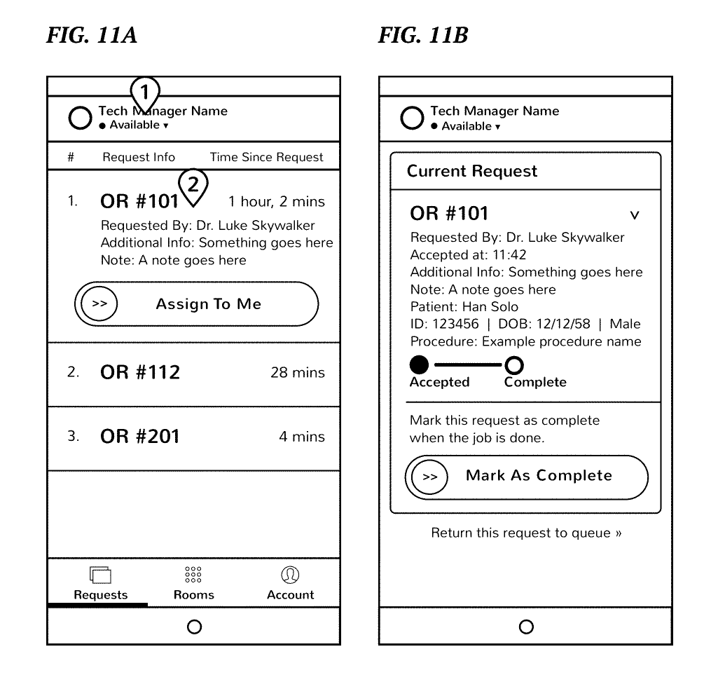

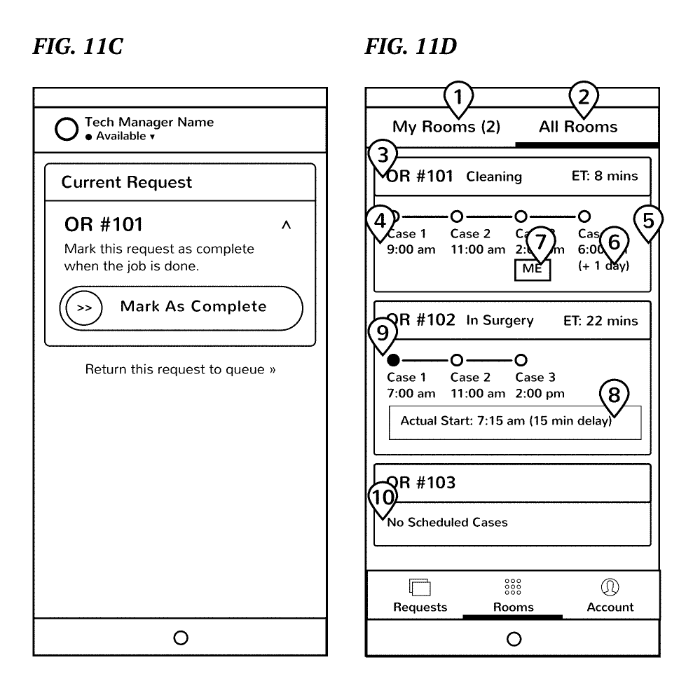

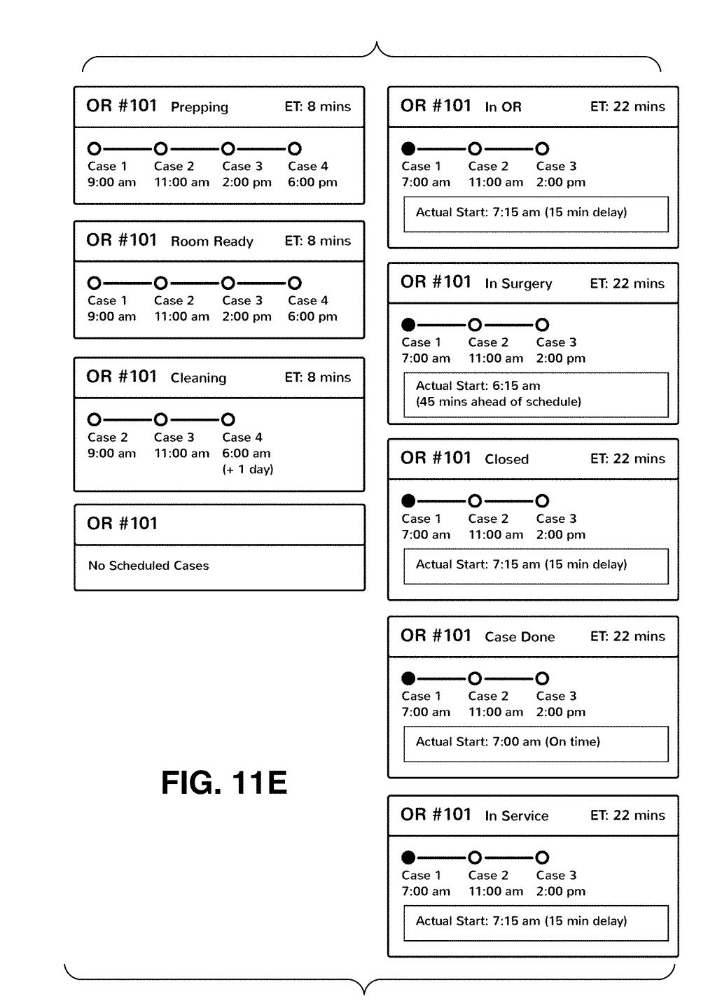

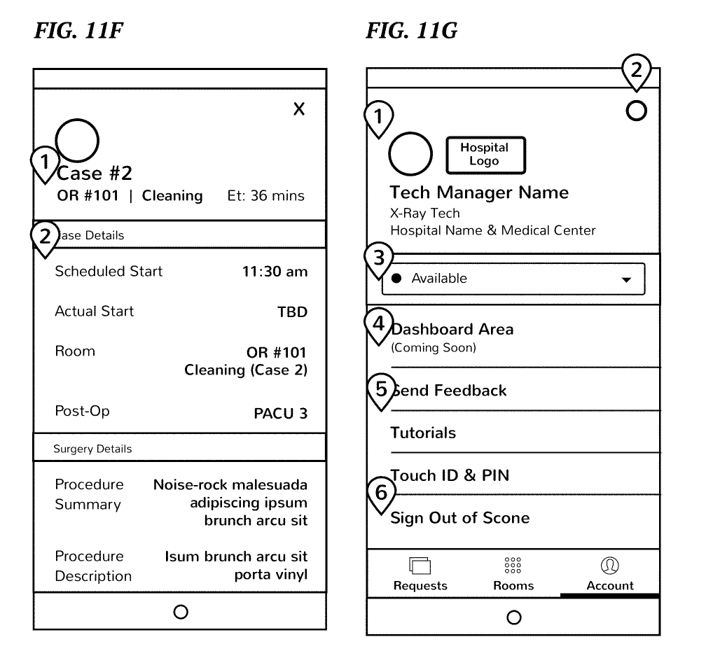

FIGS. 11A-11G depict aspects of a tech flow according to exemplary embodiments hereof.

Tech Flow—Requests

FIGS. 11A-11C depict aspects of a tech request tab (in a tech flow view) according to exemplary embodiments hereof. FIG. 11A shows a tech request tab with open requests in a list view. 1. The user may update their status directly from the status dropdown. 2. Unassigned tech request info includes: Room #, time since request, requested by, and additional info if available (added by requester via additional info dropdown). Only the request at the top of the queue may be assigned to the user. A number count of unassigned requests will appear in the navigation bar (if applicable). If the user selects a request from the list shown in FIG. 11A, they may see more details about the request (e.g., as in FIGS. 11B-11C). Note that if there are no open tech requests, the tab may state “No requests. We will notify you when the next action is needed.” or a similar message. If there is no immediate action required by the user, the tab preferably shows a waiting message.

The detail view in FIG. 11B preferably shows request details and a progress chart of the current request status; a brief description of the request, with time the request was accepted; and a primary call to action to complete the request. The user may return the request to the queue if unable to complete for any reason. The user may also cancel the request if needed. During an active request, the bottom navigation bar is preferably disabled. Note that the user is able to collapse/expand to view the request details and progress chart by selecting the up/down arrow next to the request details.

Tech Flow—Rooms

FIGS. 11D-11F depicts aspects of a Rooms tab (in a tech flow view) according to exemplary embodiments hereof. Room number will determine card order. In some implementations the room status may be one of “Prepping,” “Cleaning,” “Room Ready,” or occupied states: “In OR,” “In Surgery,” “Closed,” “Case Done,” “In Service.”

With reference to FIG. 11D: In a “My ROOMS: Card View, 1. Room cards with my cases (denoted with a “Me” badge underneath the case info) will appear in the list below. In an “ALL ROOMS: Card View,” 2. Show all room cards in the list below; 3. Room card header shows the following info: OR #, room status, room status elapsed time. The header bar color will be colored according to room status: orange=cleaning, prepping, red=occupied, green=room ready, gray=no scheduled cases; 4. Case # and scheduled time will appear in the display if available. Selecting the case # will display the room detail view (FIG. 11F); 5. The user may swipe to see more cases if needed; 6. If a displayed case is +X days in advance, the text “(in X days)” will appear. 7. My cases will be highlighted with a “Me” badge underneath the case info. 8. In OCCUPIED States (In OR, In Surgery, Closed, Case Done, In Service, OR Hold): the actual start time will be displayed for occupied rooms. The display next to the actual start will show the amount of delay or time ahead of schedule as applicable (delayed/on time/ahead of schedule); 9. For NO SCHEDULED CASES: 1. An empty state message will appear if there are no other cases scheduled for the room.

FIG. 11E depicts aspects of room card status examples in various states: “Prepping,” “Cleaning,” “Room Ready,” or occupied states: “In OR,” “In Surgery,” “Closed,” “Case Done,” “In Service.”

FIG. 11F depicts aspects of a Room Details view (in a tech flow view) according to exemplary embodiments hereof 1. The room detail header area shows the following info: Case #, room #, room status, room status elapsed time. The header background will be colored according to room status: orange=cleaning, prepping, red=occupied, green=room ready, gray=no scheduled cases; 2. Show the following info (if known): case details, surgery details, patient info (if My Patient)

Tech Flow—Account

FIG. 11G depicts aspects of an account tab (in a tech flow view) according to exemplary embodiments hereof. 1. The account header area shows the following info: user's name, photo/avatar, role, hospital name, and logo. 2. A settings link to update profile and account settings is located in the top right corner of the header area. 3. A dropdown menu allows the user to change their status (Available, Away, Do not disturb. 4. A user dashboard area provides a performance chart (e.g., weekly performance) and other data points. 5. Other links (Send feedback, tutorials, Touch ID & PIN) will take the user to more detailed views on those areas. 6. A link to sign out of the app will be listed at the bottom of the page.

Environmental Services (EVS) Flow

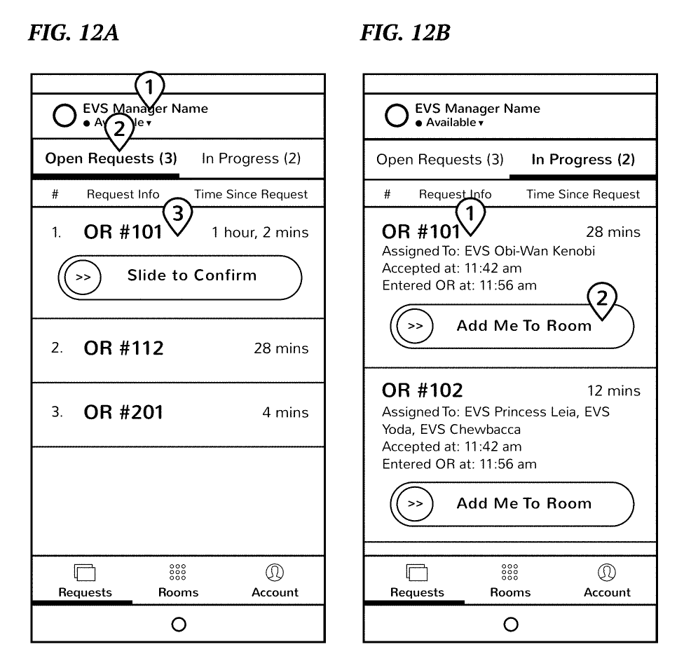

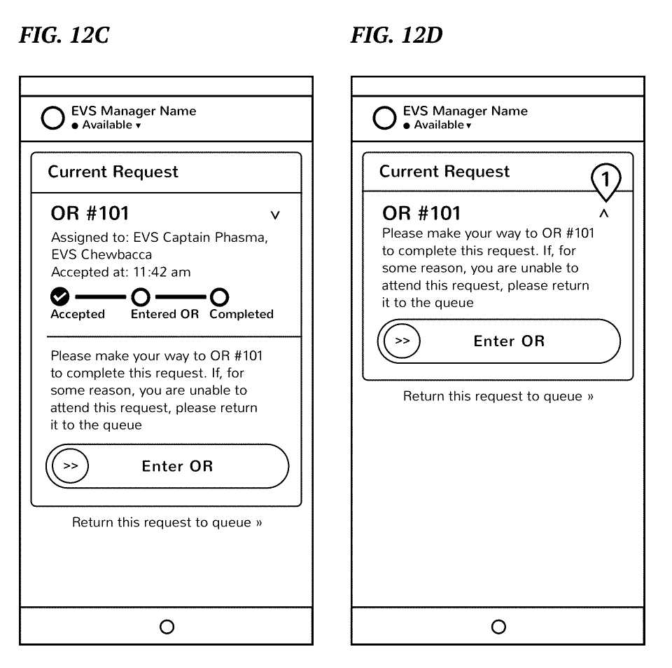

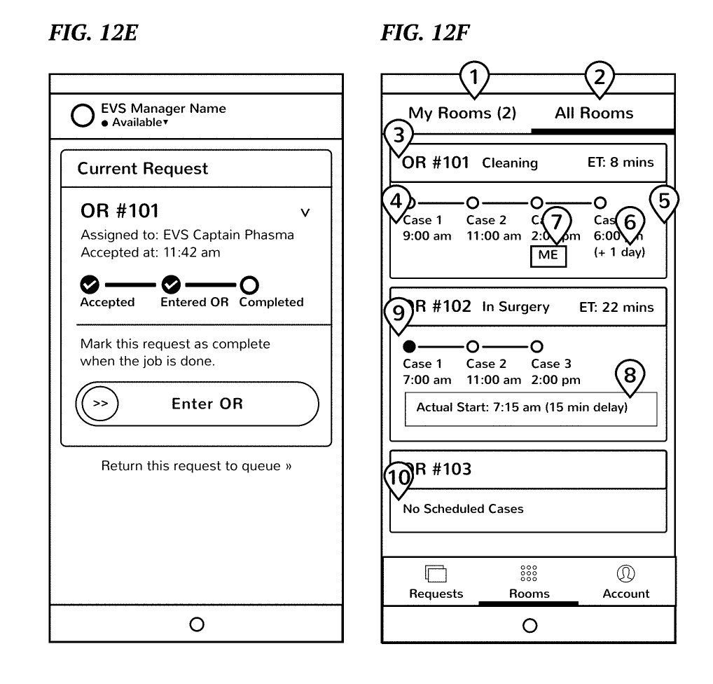

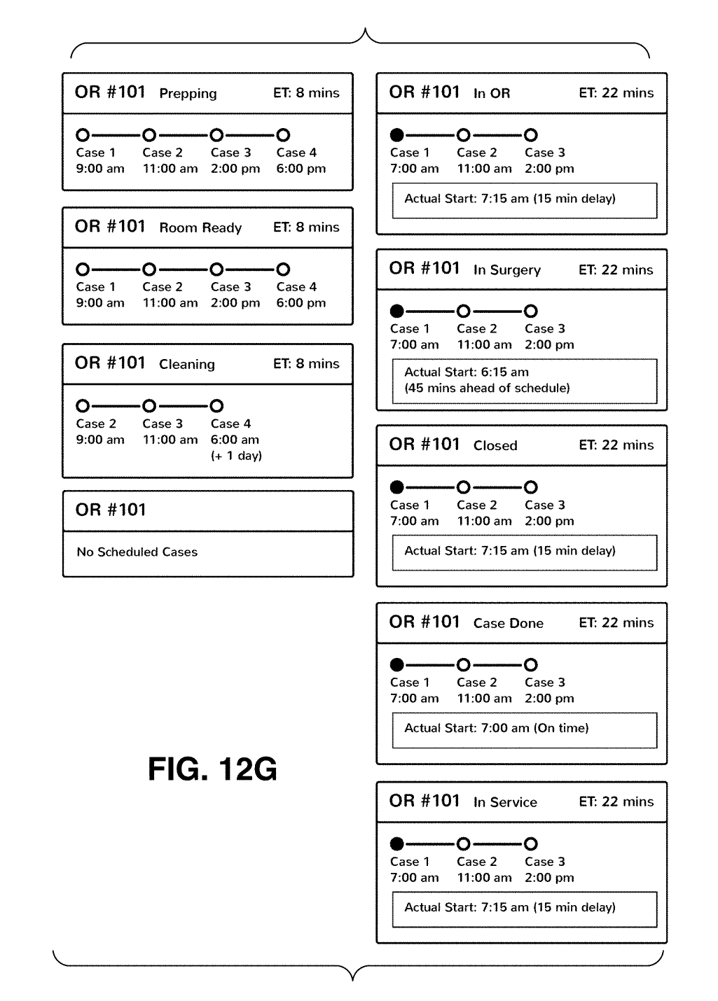

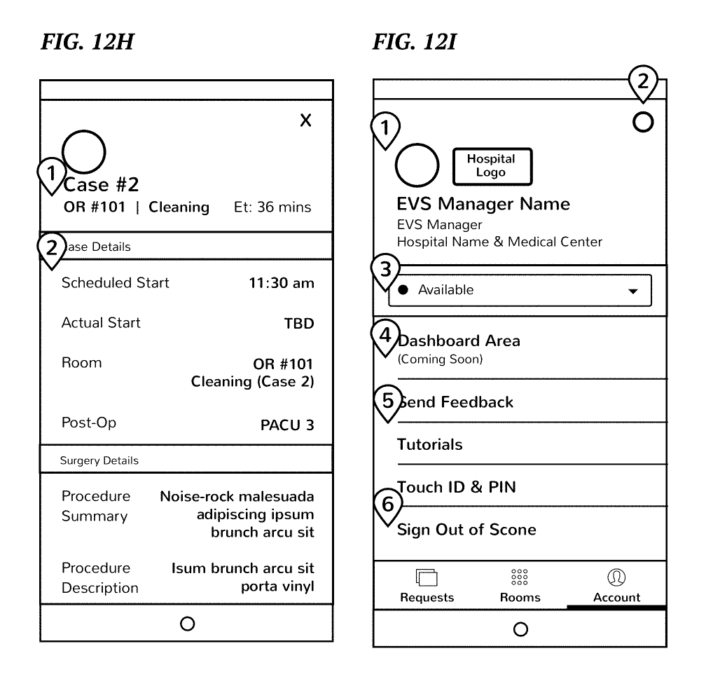

FIGS. 12A-12I depict aspects of an EVS flow according to exemplary embodiments hereof.

EVS Flow—Requests

FIGS. 12A-12C depict aspects of an EVS requests tab (in an EVS view) according to exemplary embodiments hereof

FIG. 12A shows a request tab with a list of open requests, preferably order from oldest to newest (based on time at which the request was made). 1. The user may update their status directly from the status dropdown. 2. The user may tab/switch between open/in progress requests and may select an unassigned open request. 3. An unassigned EVS request information preferably includes: location (e.g., a room number), and how long ago the request was made. In preferred embodiments the user may only select (or be assigned) the request at the top of the queue may be assigned to the user. A number count of unassigned requests may appear in the navigation bar (if applicable). Note that if there are no open EVS requests, the tab may state “No requests. We will notify you when the next action is needed.” or a similar message. If there is no immediate action required by the user, the tab preferably shows a waiting message.

If the user selects a request from the list shown in FIG. 12A, they may see more details about the request (e.g., as in FIGS. 12C-12E).

FIG. 12B depicts aspects of a tab showing requests in progress. 1. An In-Progress request preferably shows details (e.g., room number, assigned to), and progress of request, if available (e.g., time accepted, and time entered into OR). 2. A user may add themselves to an in-progress room by sliding the add button. If added, they will proceed to the request detail screen (FIG. 12C). If additional users are added, they will show up in the “assigned to” section.

A request detail screen (FIG. 12C) preferably shows request details and a progress chart of the current request status; a brief description of the request, with time the request was accepted; and a primary call to action to complete the request. If additional users are added to the room, they will be recorded in the details accordingly. The user may return the request to the queue if unable to complete for any reason. The user may also cancel the request if needed. During an active request, the bottom navigation bar is preferably disabled.

With reference to FIG. 12D: 1. User is able to collapse/expand to view the request details and progress chart. FIG. 12E shows the proceeding step in the current request.

EVS Flow—Rooms

FIGS. 12F-12H depicts aspects of a Rooms tab (in an EVS flow view) according to exemplary embodiments hereof. Room number will determine card order. In some implementations the room status may be one of “Prepping,” “Cleaning,” “Room Ready,” or occupied states: “In OR,” “In Surgery,” “Closed,” “Case Done,” “In Service.”

With reference to FIG. 12F: In a “My ROOMS: Card View, 1. Room cards with my cases (denoted with a “Me” badge underneath the case info) will appear in the list below. In an “ALL ROOMS: Card View,” 2. Show all room cards in the list below; 3. Room card header shows the following info: OR #, room status, room status elapsed time. The header bar color will be colored according to room status: orange=cleaning, prepping, red=occupied, green=room ready, gray=no scheduled cases; 4. Case # and scheduled time will appear in the display if available. Selecting the case # will display the room detail view (FIG. 12H); 5. The user may swipe to see more cases if needed; 6. If a displayed case is +X days in advance, the text “(in X days)” will appear. 7. My cases will be highlighted with a “Me” badge underneath the case info. 8. In OCCUPIED States (In OR, In Surgery, Closed, Case Done, In Service, OR Hold): the actual start time will be displayed for occupied rooms. The display next to the actual start will show the amount of delay or time ahead of schedule as applicable (delayed/on time/ahead of schedule); 9. For NO SCHEDULED CASES: 1. An empty state message will appear if there are no other cases scheduled for the room.

FIG. 12G depicts aspects of room card status examples in various states: “Prepping,” “Cleaning,” “Room Ready,” or occupied states: “In OR,” “In Surgery,” “Closed,” “Case Done,” “In Service.”

FIG. 12H depicts aspects of a Room Details view (in an EVS flow view) according to exemplary embodiments hereof. 1. The room detail header area shows the following info: Case #, room #, room status, room status elapsed time. The header background will be colored according to room status: orange=cleaning, prepping, red=occupied, green=room ready, gray=no scheduled cases; 2. Show the following info (if known): case details, surgery details, patient info (if My Patient)

EVS Flow—Account

FIG. 12I depicts aspects of an account tab (in an EVS flow view) according to exemplary embodiments hereof. 1. The account header area shows the following info: user's name, photo/avatar, role, hospital name, and logo. 2. A settings link to update profile and account settings is located in the top right corner of the header area. 3. A dropdown menu allows the user to change their status (Available, Away, Do not disturb) 4. A user dashboard area provides a performance chart (e.g., weekly performance) and other data points. 5. Other links (Send feedback, tutorials, Touch ID & PIN) will take the user to more detailed views on those areas. 6. A link to sign out of the app will be listed at the bottom of the page.

Accordingly, in some aspects, exemplary embodiments hereof provide a system supporting perioperative workflow. The system may include a backend system and one or more devices. The backend system may include at least one database; at least one application configured with the at least one database; and at least one user interface mechanism supporting a plurality of role-specific graphical user interfaces (GUIs) to the backend system.

In some aspects, the backend system maintains in the at least one database, perioperative workflow information for each of a plurality of patients, wherein the perioperative workflow information maintained by the backend system is an authoritative version of the perioperative workflow information.

In some aspects, the one or more devices may be configured with at least some of the plurality of role-specific GUIs, and may be operably connected to the backend system and may interact with the backend system via the at least one user interface mechanism.

In some aspects, each particular device of may be configured: to receive and display perioperative workflow information from the backend system in the role-specific GUI on the particular device; and to send perioperative workflow information to the backend system via the role-specific GUI on the particular device.

In some other aspects, exemplary embodiments hereof provide a method, in a system comprising: a backend system having: at least one database; at least one application configured with the at least one database; and at least one user interface mechanism supporting a plurality of role-specific graphical user interfaces (GUIs) to the backend system. The system also has one or more devices configured with at least some of the plurality of role-specific GUIs, and operably connected to the backend system and interacting with the backend system via the at least one user interface mechanism, wherein each particular device of the one or more devices may be configured to receive and display perioperative workflow information from the backend system in the role-specific GUI on the particular device; and to send perioperative workflow information to the backend system via the role-specific GUI on the particular device.

The exemplary method includes: maintaining in the at least one database, perioperative workflow information for each of a plurality of patients, and wherein the perioperative workflow information maintained by the backend system is an authoritative version of the perioperative workflow information. The method may further include, for each specific patient of the plurality of patients, monitoring the specific patient's flow through the perioperative workflow associated with the specific patient; and adjusting the perioperative workflow associated with the specific patient based on: perioperative workflow information maintained in the at least one database at the backend system; and perioperative workflow information modified or deleted via the role-specific GUIs on the one or more devices.

In some aspects, the perioperative workflow information in the at least one database may include a sequence of perioperative workflow steps.

In some aspects, the sequence of perioperative workflow steps may include synchronous steps and asynchronous steps.

In some aspects, the system preferably supports a plurality of user roles, wherein each particular user role has a corresponding role-specific GUI associated therewith.

In some aspects, the role-specific GUI associated with each particular user role may provide role-specific capabilities and role-specific permissions, and wherein the backend system enforces the role-specific capabilities and the role-specific permissions via the role-specific GUIs.

In some aspects, the role-specific capabilities and the role-specific permissions may include permission to view certain perioperative workflow information; and permission to modify or delete certain perioperative workflow information.

In some aspects, for certain roles, the permission to view certain perioperative workflow information may comprise permission to view certain perioperative workflow information associated with one or more specific patients.

In some aspects, for certain roles, the permission to modify or delete certain perioperative workflow information may comprise permission to modify or delete certain perioperative workflow information associated with one or more specific patients.

In some aspects, the plurality of user roles are selected from the group:

administrator, service manager, non-operating room (OR) nurse, doctor, anesthesiologist, OR nurse, tech, and environmental services.

In some aspects, each specific patient of the plurality of patients has a corresponding specific perioperative workflow.

In some aspects, the perioperative workflow associated with each particular patient may be initially based on an expected treatment or procedure for the particular patient.

In some aspects, the at least one application may comprise a scheduling application and a workflow application, and wherein, for each specific patient of the plurality of patients, the scheduling application and the workflow application: (a) monitor the specific patient's flow through the perioperative workflow associated with the specific patient; and (b) adjust the perioperative workflow associated with the specific patient based on: (b)(1) perioperative workflow information maintained in the at least one database at the backend system; and (b)(2) perioperative workflow information modified or deleted via the role-specific GUIs on the one or more devices.

In some aspects, the perioperative workflow associated with the specific patient may comprise synchronous steps and asynchronous steps.

In some aspects, the scheduling application and the workflow application may adjust to real-time variability of each step in the perioperative workflow associated with the specific patient.

In some aspects, the at least some of the role-specific GUIs may provide a real-time view into steps within the perioperative workflow associated with the specific patient.

In some aspects, the sequence of perioperative workflow steps may be selected from: user registration, user login, patient information entry, viewing patient information, viewing patient status, patient scheduling, doctor information entry, viewing doctor information, doctor scheduling, requesting doctor, procedure information entry, viewing procedure information, scheduling procedure, requesting procedure, non-operating room (OR) nurse information entry, viewing non-OR nurse information, scheduling non-OR nurse, requesting non-OR nurse, OR nurse information entry, viewing OR nurse information, scheduling OR nurse, requesting OR nurse, anesthesiologist information entry, viewing anesthesiologist information, scheduling anesthesiologist, requesting anesthesiologist, tech information entry, viewing tech information, scheduling tech, requesting tech, tech request information entry, viewing tech request information, scheduling tech request, environmental services information entry, viewing environmental services information, scheduling environmental services, requesting environmental services, requesting blood, lab information, transport information, and room scheduling.

In some aspects, the role-specific permissions may be selected from: administrator permissions, service manager permissions, non-OR nurse permissions, doctor permissions, anesthesiologist permissions, OR nurse permissions, tech permissions, and environmental services permissions.

In some aspects, the at least one application may comprise: a data evaluation application configured to analyze at least one perioperative workflow and to generate at least one report based on the analysis.

In some aspects, the at least one report may be used to modify aspects of the at least one perioperative workflow.

In some aspects, the aspects of the at least one perioperative workflow may include at least one sequence of perioperative workflow steps.

In some aspects, the at least one application may comprise one or more of: a configuration application, an administration application, a perioperative workflow scheduling application, a perioperative workflow application, an intake application, an output application, and a data evaluation application.

In some aspects, the at least one database may comprise one or more of: a perioperative workflow scheduling database, a configuration database, a general and administrative database, and a perioperative workflow information database.

In some aspects, each role-specific GUI may display perioperative workflow information in a corresponding role-specific manner.

In some aspects, displayed perioperative workflow information may comprise one or more of: user information, login information, patient information, room information, doctor information, service request information, report information, tech request information, lab information, transport information, and blood request information.

In some aspects, when a user role is administrator, the at least one role-specific GUI may include an administrative GUI that sends certain perioperative workflow information to the backend system.

In some aspects, the certain perioperative workflow information may be selected from: user detail information, login information, patients detail information, room information, doctors information, service request information, report information, tech request information, lab information, transport information, and blood request information.

In some aspects, the at least one role-specific GUI may include a service manager flow GUI that displays certain perioperative workflow information received from the backend system.

In some aspects, the displayed perioperative workflow information may be selected from: service requests information, room information, and account information.

In some aspects, the at least one role-specific GUI may include a service manager GUI that sends perioperative workflow information to the backend system.

In some aspects, the sent perioperative workflow information may be selected from service request information, rooms information, and accounts information.

In some aspects, the at least one role-specific GUI may include a non-OR nurse flow GUI that displays perioperative workflow information received from the backend system.

In some aspects, the displayed perioperative workflow information may be selected from: login information, patient information, history information, request information, room information, and account information.

In some aspects, the at least one role-specific GUI may include a non-OR nurse flow GUI that sends certain perioperative workflow information to the backend system.

In some aspects, the certain perioperative workflow information may be selected from: login information, patient information, history information, request information, rooms information, and account information.

In some aspects, the at least one role-specific GUI may be selected from: a doctor flow GUI, an anesthesiologist flow GUI and an OR nurse flow GUI, that each display certain perioperative workflow information received from the backend system.

In some aspects, the certain perioperative workflow information may be selected from: patient information, procedure information, room information, service request information, tech request information, blood request information, history information, and account information.

In some aspects, the at least one role-specific GUI may be selected from: a doctor flow GUI, an anesthesiologist flow GUI, and an OR nurse flow interface, each of which sends certain perioperative workflow information to the backend system.

In some aspects, the certain perioperative workflow information may be selected from: patient information, procedure information, room information, service request information, tech request information, blood request information, history information, and accounts information.

In some aspects, the at least one role-specific GUI may include a tech flow GUI that displays certain perioperative workflow information received from the backend system and sends perioperative workflow information to the backend system.

In some aspects, the certain perioperative workflow information displayed by the tech flow GUI may be selected from: tech request information, room information, and account information.

In some aspects, the at least one role-specific GUI may include an environmental services flow GUI that displays certain perioperative workflow information received from the backend system and sends perioperative workflow information to the backend system.

In some aspects, the displayed certain perioperative workflow information may be selected from: request information, room information, and account information.

In some aspects, the at least one application may include an intake application configured to receive information from an external system, and an output application configured to send information to an external system.

In some aspects, the backend system may be configured to generate reports based on stored perioperative information.

In some aspects, the backend system may be configured to determine efficiency of a particular perioperative process.

In some aspects, the at least one application may be configured to track synchronous and asynchronous steps required in the sequence associated with a particular perioperative workflow associated with a particular patient.

In some aspects, the at least one application may monitor the particular patient flow through the system.

In some other aspects, the each of the one or more devices may be selected from: a mobile phone, a tablet computer, a desktop computer, and a laptop computer.

### Computing

The services, mechanisms, operations and acts shown and described above are implemented, at least in part, by software running on one or more computers or computer systems or devices. It should be appreciated that each user device is, or comprises, a computer system.

Programs that implement such methods (as well as other types of data) may be stored and transmitted using a variety of media (e.g., computer readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general purpose computers, special purpose computers and computing devices. One or more such computers or computing devices may be referred to as a computer system.

FIG. 4A is a schematic diagram of a computer system 400 upon which embodiments of the present disclosure may be implemented and carried out.

According to the present example, the computer system 400 includes a bus 402 (i.e., interconnect), one or more processors 404, one or more communications ports 414, a main memory 406, removable storage media 410, read-only memory 408, and a mass storage 412. Communication port(s) 414 may be connected to one or more networks by way of which the computer system 400 may receive and/or transmit data.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 404 can be (or include) any known processor, such as, but not limited to, an Intel® Itanium® or Itanium 2® processor(s), AMD® Opteron® or Athlon MP® processor(s), or Motorola® lines of processors, and the like. Communications port(s) 414 can be any of an RS-232 port for use with a modem based dial-up connection, a 10/100 Ethernet port, a Gigabit port using copper or fiber, or a USB port, and the like. Communications port(s) 414 may be chosen depending on a network such as a Local Area Network (LAN), a Wide Area Network (WAN), a CDN, or any network to which the computer system 400 connects. The computer system 400 may be in communication with peripheral devices (e.g., display screen 416, input device(s) 418) via Input/Output (I/O) port 420. Some or all of the peripheral devices may be integrated into the computer system 400, and the input device(s) 418 may be integrated into the display screen 416 (e.g., in the case of a touch screen).

Main memory 406 can be Random Access Memory (RAM), or any other dynamic storage device(s) commonly known in the art. Read-only memory 408 can be any static storage device(s) such as Programmable Read-Only Memory (PROM) chips for storing static information such as instructions for processor(s) 404. Mass storage 412 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 402 communicatively couples processor(s) 404 with the other memory, storage and communications blocks. Bus 402 can be a PCI/PCI-X, SCSI, a Universal Serial Bus (USB) based system bus (or other) depending on the storage devices used, and the like. Removable storage media 410 can be any kind of external hard-drives, floppy drives, IOMEGA® Zip Drives, Compact Disc-Read Only Memory (CD-ROM), Compact Disc-Re-Writable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory, which typically constitutes the main memory of the computer. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols; and/or (iv) encrypted in any of a variety of ways well known in the art.

A computer-readable medium can store (in any appropriate format) those program elements that are appropriate to perform the methods.

As shown, main memory 406 is encoded with application(s) 422 that support(s) the functionality as discussed herein (an application 422 may be an application that provides some or all of the functionality of one or more of the mechanisms described herein). Application(s) 422 (and/or other resources as described herein) can be embodied as software code such as data and/or logic instructions (e.g., code stored in the memory or on another computer readable medium such as a disk) that supports processing functionality according to different embodiments described herein.

For example, as shown in FIGS. 4B and 4C, respectively, application(s) 422 may include device application(s) 422-1 in FIG. 4B (corresponding to 302 in FIG. 3A), and backend application(s) 422-2 in FIG. 4B (corresponding to applications 110 in FIG. 2, and corresponding to backend service(s)).

During operation of some embodiments, processor(s) 404 accesses main memory 406 via the use of bus 402 in order to launch, run, execute, interpret, or otherwise perform the logic instructions of the application(s) 422. Execution of application(s) 422 produces processing functionality of the service(s) or mechanism(s) related to the application(s). In other words, the process(es) 424 represents one or more portions of the application(s) 422 performing within or upon the processor(s) 404 in the computer system 400.

For example, as shown in FIG. 4D, process(es) 424 may include device process(es) 424-1, corresponding to one or more of the device application(s) 422-1. Similarly, as shown in FIG. 4E, process(es) 424 may include backend process(es) 424-2, corresponding to one or more of the backend application(s) 422-2.

It should be noted that, in addition to the process(es) 424 that carries(carry) out operations as discussed herein, other embodiments herein include the application 422 itself (i.e., the un-executed or non-performing logic instructions and/or data). The application 422 may be stored on a computer readable medium (e.g., a repository) such as a disk or in an optical medium. According to other embodiments, the application 422 can also be stored in a memory type system such as in firmware, read only memory (ROM), or, as in this example, as executable code within the main memory 406 (e.g., within Random Access Memory or RAM). For example, application 422 may also be stored in removable storage media 410, read-only memory 408, and/or mass storage device 412.

Those skilled in the art will understand that the computer system 400 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In other embodiments, the process includes some human intervention (e.g., a step is performed by or with the assistance of a human).

Real-Time

Those of ordinary skill in the art will realize and understand, upon reading this description, that, as used herein, the term “real time” means near real time or sufficiently real time. It should be appreciated that there are inherent delays in network-based communication (e.g., based on network traffic and distances), and these delays may cause delays in data reaching various components. Inherent delays in the system do not change the real time nature of the data. In some cases, the term “real time data” may refer to data obtained in sufficient time to make the data useful for its intended purpose.

Although the term “real time” may be used here, it should be appreciated that the system is not limited by this term or by how much time is actually taken. In some cases, real time computation may refer to an online computation, i.e., a computation that produces its answer(s) as data arrive, and generally keeps up with continuously arriving data. The term “online” computation is compared to an “offline” or “batch” computation.

As used in this description, the term “portion” means some or all. Therefore, for example, “A portion of X” may include some of “X” or all of “X”. In the context of a conversation, the term “portion” means some or all of the conversation.

As used herein, including in the claims, the phrase “at least some” means “one or more,” and includes the case of only one. Thus, e.g., the phrase “at least some ABCs” means “one or more ABCs,” and includes the case of only one ABC.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only,” the phrase “based on X” does not mean “based only on X.”

As used herein, including in the claims, the phrase “using” means “using at least,” and is not exclusive. Thus, e.g., the phrase “using X” means “using at least X.” Unless specifically stated by use of the word “only,” the phrase “using X” does not mean “using only X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a),” “(b),” and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram the activities associated with those boxes may be performed in any order, including fully or partially in parallel.

Thus are described systems, methods, and devices that will transform hospital productivity and efficiency through a real-time logistics platform powering action-based, intuitive, adaptive-view mobile applications that modernize how standard workflows are implemented, tracked, and analyzed.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed:

1. A system supporting perioperative workflow, the system comprising:
(A) a backend system comprising:
(A)(1) at least one database;
(A)(2) at least one application configured with said at least one database; and
(A)(3) at least one user interface mechanism supporting a plurality of role-specific graphical user interfaces (GUIs) to said backend system, wherein the backend system maintains in said at least one database, perioperative workflow information for each of a plurality of patients, and wherein the perioperative workflow information maintained by the backend system is an authoritative version of the perioperative workflow information; and
(B) one or more devices configured with at least some of the plurality of role-specific GUIs, and operably connected to said backend system and interacting with said backend system via said at least one user interface mechanism,
wherein a particular device of said one or more devices is configured to:
(B)(1) receive and display perioperative workflow information from said backend system in a particular role-specific GUI on said particular device; and
(B)(2) send perioperative workflow information to said backend system via said particular role-specific GUI on said particular device.

2. The system of claim 1, wherein said perioperative workflow information in said at least one database comprises a sequence of perioperative workflow steps, wherein said sequence of perioperative workflow steps comprise synchronous steps and/or asynchronous steps,
wherein each specific patient of said plurality of patients has a corresponding specific perioperative workflow, wherein
the perioperative workflow associated with a particular patient of said plurality of patients is initially based on an expected treatment or procedure for said particular patient.

3. The system of claim 2, wherein said at least one application is configured to track synchronous and asynchronous steps in the sequence associated with a particular perioperative workflow associated with a particular patient.

4. The system of claim 3, wherein said at least one application comprises a scheduling application and a workflow application, and wherein, for a specific patient of said plurality of patients, said scheduling application and said workflow application:
(a) monitor said specific patient's flow through the perioperative workflow associated with said specific patient; and
(b) adjust said perioperative workflow associated with said specific patient based on:
(b)(1) perioperative workflow information maintained in said at least one database at said backend system; and/or
(b)(2) perioperative workflow information modified or deleted via said role-specific GUIs on said one or more devices.

5. The system of claim 4, wherein said perioperative workflow associated with said specific patient comprises synchronous steps and asynchronous steps.

6. The system of claim 4, wherein the scheduling application and the workflow application adjust to real-time variability of at least one step in said perioperative workflow associated with said specific patient.

7. The system of claim 4, wherein at least some of the role-specific GUIs provide a real-time view into steps within the perioperative workflow associated with said specific patient.

8. The system of claim 1, wherein said system supports a plurality of user roles, and wherein each particular user role has a corresponding role-specific GUI associated therewith.

9. The system of claim 8, wherein the corresponding role-specific GUI associated with a particular user role provides role-specific capabilities and role-specific permissions, and wherein said backend system enforces said role-specific capabilities and said role-specific permissions via said role-specific GUIs.

10. The system of claim 9, wherein the role-specific capabilities and the role-specific permissions include: permission to view certain perioperative workflow information; and permission to modify or delete certain perioperative workflow information.

11. The system of claim 10, wherein, for certain roles, said permission to view certain perioperative workflow information comprises permission to view certain perioperative workflow information associated with one or more specific patients.

12. The system of claim 10, wherein, for certain roles, said permission to modify or delete certain perioperative workflow information comprises permission to modify or delete certain perioperative workflow information associated with one or more specific patients.

13. The system of claim 1, wherein said at least one application comprises:
a data evaluation application configured to perform an analysis of at least one perioperative workflow and to generate at least one report based on the analysis.

14. The system of claim 13, wherein aspects of said at least one perioperative workflow are modified based on said analysis.

15. The system of claim 1, wherein each role-specific GUI displays perioperative workflow information in a corresponding role-specific manner.

16. The system of claim 1, wherein said at least one application includes an intake application configured to receive information from an external system, and an output application configured to send information to an external system.

17. The system of claim 1, wherein the backend system is configured to determine efficiency of a particular perioperative process.

18. The system of claim 1, wherein said one or more devices are selected from a group comprising: a mobile phone, a tablet computer, a desktop computer, and a laptop computer.

19. A method, in a system comprising:
(A) a backend system having:
(A)(1) at least one database;
(A)(2) at least one application configured with said at least one database; and
(A)(3) at least one user interface mechanism supporting a plurality of role-specific graphical user interfaces (GUIs) to said backend system,
(B) one or more devices configured with at least some of the plurality of role-specific GUIs, and operably connected to said backend system and interacting with said backend system via said at least one user interface mechanism,
wherein a particular device of said one or more devices is configured to:
(B)(1) receive and display perioperative workflow information from said backend system in a particular role-specific GUI on said particular device; and
(B)(2) send perioperative workflow information to said backend system via said particular role-specific GUI on said particular device,
the method comprising:
(a) maintaining in said at least one database, perioperative workflow information for each of a plurality of patients, and wherein the perioperative workflow information maintained by the backend system is an authoritative version of the perioperative workflow information; and
(b) for a specific patient of said plurality of patients,
(b)(1) monitoring said specific patient's flow through the perioperative workflow associated with said specific patient; and
(b)(2) adjusting said perioperative workflow associated with said specific patient based on:
(b)(2)(i) perioperative workflow information maintained in said at least one database at said backend system; and/or
(b)(2)(ii) perioperative workflow information modified or deleted via said role-specific GUIs on said one or more devices.

20. A non-transitory computer-readable medium with one or more computer programs stored therein that, when executed by one or more processors in a system comprising: (A) a backend system having: (A)(1) at least one database; (A)(2) at least one application configured with said at least one database; and (A)(3) at least one user interface mechanism supporting a plurality of role-specific graphical user interfaces (GUIs) to said backend system,
cause the one or more processors to perform at least the operations of:
(a) maintaining in said at least one database, perioperative workflow information for each of a plurality of patients, and wherein the perioperative workflow information maintained by the backend system is an authoritative version of the perioperative workflow information; and
(b) for a specific patient of said plurality of patients,
(b)(1) monitoring said specific patient's flow through the perioperative workflow associated with said specific patient; and
(b)(2) adjusting said perioperative workflow associated with said specific patient based on:
(b)(2)(i) perioperative workflow information maintained in said at least one database at said backend system; and/or
(b)(2)(ii) perioperative workflow information modified or deleted via said role-specific GUIs on said one or more devices.
